(19) United States
(12) Patent Application Publication
(10) Pub. No.: US 2013/0269063 A1
(43) Pub. Date: Oct. 10, 2013

(54) DROUGHT TOLERANT PLANTS AND RELATED CONSTRUCTS AND METHODS INVOLVING GENES ENCODING MATE-EFFLUX POLYPEPTIDES

(71) Applicants: Stephen M. Allen, Wilmington, DE (US); Stanley Luck, Wilmington, DE (US); Jeffrey Mullen, Maple Plain, MN (US); Hajime Sakai, Newark, DE (US); Scott V. Tingey, Wilmington, DE (US); Robert Wayne Williams, Hockessin, DE (US); Norbert Brugiere, Johnston, IA (US); Ratna Kumria, Hyderabad (IN); Amitabh Mohanty, Secunderaba (IN); Rupa Raja, Secunderabad (IN)

(72) Inventors: Stephen M. Allen, Wilmington, DE (US); Stanley Luck, Wilmington, DE (US); Jeffrey Mullen, Maple Plain, MN (US); Hajime Sakai, Newark, DE (US); Scott V. Tingey, Wilmington, DE (US); Robert Wayne Williams, Hockessin, DE (US); Norbert Brugiere, Johnston, IA (US); Ratna Kumria, Hyderabad (IN); Amitabh Mohanty, Secunderaba (IN); Rupa Raja, Secunderabad (IN)

(73) Assignees: PIONEER HI BRED INTERNATIONAL INC, WILMINGTON, DE (US); E I DU PONT DE NEMOURS AND COMPANY, WILMINGTON, DE (US)

(21) Appl. No.: 13/989,838

(22) PCT Filed: Dec. 19, 2011
(86) PCT No.: PCT/US11/65779

§ 371 Date: May 28, 2013

Related U.S. Application Data
(60) Provisional application No. 61/424,936, filed on Dec. 20, 2010.

(51) Int. Cl. C12N 15/82

(52) U.S. Cl. CPC C12N 15/8261; C12N 15/8271; C12N 15/8273
USPC 800/289; 800/298; 800/320.1; 800/312; 800/322; 800/306; 800/320; 800/320.3; 800/314; 800/320.2; 800/278; 536/23.6

(57) ABSTRACT

Isolated polynucleotides and polypeptides and recombinant DNA constructs useful for conferring drought tolerance, compositions (such as plants or seeds) comprising these recombinant DNA constructs, and methods utilizing these recombinant DNA constructs. The recombinant DNA construct comprises a polynucleotide operably linked to a promoter that is functional in a plant, wherein said polynucleotide encodes a MATE-efflux polypeptide.

### CROSS REFERENCE TO RELATED APPLICATIONS

This application claims the benefit of U.S. Provisional Application No. 61/424,936, filed Dec. 20, 2011, the entire content of which is herein incorporated by reference.

### FIELD OF THE INVENTION

The field of invention relates to plant breeding and genetics and, in particular, relates to recombinant DNA constructs useful in plants for conferring tolerance to drought.

### BACKGROUND OF THE INVENTION

Abiotic stress is the primary cause of crop loss worldwide, causing average yield losses of more than 50% for major crops (Boyer, J. S. (1982) Science 218:443-448; Bray, E. A. et al. (2000) In Biochemistry and Molecular Biology of Plants, Edited by Buchannan, B. B. et al., Amer. Soc. Plant Biol., pp. 1158-1203). Among the various abiotic stresses, drought is the major factor that limits crop productivity worldwide. Exposure of plants to a water-limiting environment during various developmental stages appears to activate various physiological and developmental changes. Understanding of the basic biochemical and molecular mechanism for drought stress perception, transduction and tolerance is a major challenge in biology. Reviews on the molecular mechanisms of abiotic stress responses and the genetic regulatory networks of drought stress tolerance have been published (Valllyodan, B., and Nguyen, H. T., (2006) Curr. Opin. Plant Biol. 9:189-195; Wang, W., et al. (2003) Planta 218:1-14); Vinocur, B., and Altman, A. (2005) Curr. Opin. Biotechnol. 16:123-132; Chaves, M. M., and Oliveira, M. M. (2004) J. Exp. Bot. 55:2365-2384; Shinozaki, K., et al. (2003) Curr. Opin. Plant Biol. 6:410-417; Yamaguchi-Shinozaki, K., and Shinozaki, K. (2005) Trends Plant Sci. 10:88-94).

Earlier work on molecular aspects of abiotic stress responses was accomplished by differential and/or subtractive analysis (Bray, E. A. (1993) Plant Physiol. 103:1035-1040; Shinozaki, K., and Yamaguchi-Shinozaki, K. (1997) Plant Physiol. 115:327-334: Zhu, J.-K. et al. (1997) Crit. Rev. Plant Sci. 16:253-277; Thomashow, M. F. (1999) Annu. Rev. Plant Physiol. Plant Mol. Biol. 50:571-599). Other methods include selection of candidate genes and analyzing expression of such a gene or its active product under stresses, or by functional complementation in a stressor system that is well defined (Xiong, L., and Zhu, J.-K. (2001) Physiologia Plantarum 112:152-166). Additionally, forward and reverse genetic studies involving the identification and isolation of mutations in regulatory genes have also been used to provide evidence for observed changes in gene expression under stress or exposure (Xiong, L., and Zhu, J.-K. (2001) Physiologia Plantarum 112:152-166).

Activation tagging can be utilized to identify genes with the ability to affect a trait. This approach has been used in the model plant species Arabidopsis thaliana (Weigel, D., et al. (2000) Plant Physiol. 122:1003-1013). Insertions of transcriptional enhancer elements can dominantly activate and/or elevate the expression of nearby endogenous genes. This method can be used to select genes involved in agronomically important phenotypes, including stress tolerance.

### SUMMARY OF THE INVENTION

The present invention includes:

In one embodiment, a plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, and wherein said plant exhibits either increased drought tolerance, increased osmotic stress tolerance, or both, when compared to a control plant not comprising said recombinant DNA construct.

In another embodiment, a plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, and wherein said plant exhibits an alteration of at least one agronomic characteristic when compared to a control plant not comprising said recombinant DNA construct. Optionally, the plant exhibits said alteration of said at least one agronomic characteristic when compared, under water limiting conditions, to said control plant not comprising said recombinant DNA construct. The at least one agronomic trait may be yield, biomass, or both and the alteration may be an increase.

In one embodiment, a plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, and wherein said plant exhibits increased tolerance to osmotic stress when compared to a control plant not comprising said recombinant DNA construct.

In another embodiment, the present invention includes any of the plants of the present invention wherein the plant is selected from the group consisting of: Arabidopsis, maize, soybean, sunflower, sorghum, canola, wheat, alfalfa, cotton, rice, barley, millet, sugar cane and switchgrass.

In another embodiment, the present invention includes seed of any of the plants of the present invention, wherein said seed comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, and wherein a plant produced from said seed exhibits either an increase in at least one trait selected from the group consisting of: drought tolerance, osmotic stress tolerance, yield and biomass, when compared to a control plant not comprising said recombinant DNA construct.

In another embodiment, a method of increasing drought tolerance in a plant, comprising: (a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) regenerating a transgenic plant from the regenerable plant cell after step (a), wherein the transgenic plant comprises in its genome the recombinant DNA construct; and (c) obtaining a progeny plant derived from the transgenic plant of step (b), wherein said progeny plant comprises in its genome the recombinant DNA construct and exhibits increased drought tolerance when compared to a control plant not comprising the recombinant DNA construct.

In another embodiment, a method of increasing osmotic stress tolerance in a plant, comprising: (a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) regenerating a transgenic plant from the regenerable plant cell after step (a), wherein the transgenic plant comprises in its genome the recombinant DNA construct; and (c) obtaining a progeny plant derived from the transgenic plant of step (b), wherein said progeny plant comprises in its genome the recombinant DNA construct and exhibits increased osmotic stress tolerance when compared to a control plant not comprising the recombinant DNA construct.

In another embodiment, a method of evaluating drought tolerance in a plant, comprising: (a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) obtaining a progeny plant derived from the transgenic plant, wherein the progeny plant comprises in its genome the recombinant DNA construct; and (c) evaluating the progeny plant for drought tolerance compared to a control plant not comprising the recombinant DNA construct.

In another embodiment, a method of increasing abiotic stress tolerance in a plant, comprising: (a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) regenerating a transgenic plant from the regenerable plant cell after step (a), wherein the transgenic plant comprises in its genome the recombinant DNA construct; and (c) obtaining a progeny plant derived from the transgenic plant of step (b), wherein said progeny plant comprises in its genome the recombinant DNA construct and exhibits increased tolerance to at least one abiotic stress selected from the group consisting of drought stress, osmotic stress, heat stress, high light stress, salt stress, paraquat stress and cold temperature stress, when compared to a control plant not comprising the recombinant DNA construct.

In another embodiment, a method of determining an alteration of at least one agronomic characteristic in a plant, comprising: (a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, wherein the transgenic plant comprises in its genome the recombinant DNA construct; (c) obtaining a progeny plant derived from the transgenic plant, wherein the progeny plant comprises in its genome the recombinant DNA construct; and (d) determining whether the progeny plant exhibits an alteration of at least one agronomic characteristic when compared to a control plant not comprising the recombinant DNA construct. Optionally, said determining step (d) comprises determining whether the transgenic plant exhibits an alteration of at least one agronomic characteristic when compared, under water limiting conditions, to a control plant not comprising the recombinant DNA construct. The at least one agronomic trait may be yield, biomass, or both and the alteration may be an increase.

In another embodiment, the present invention includes any of the methods of the present invention wherein the plant is selected from the group consisting of: Arabidopsis, maize, soybean, sunflower, sorghum, canola, wheat, alfalfa, cotton, rice, barley, millet, sugar cane and switchgrass.

In another embodiment, the present invention includes an isolated polynucleotide comprising: (a) a nucleotide sequence encoding a polypeptide with drought tolerance activity, wherein the polypeptide has an amino acid sequence of at least 90% sequence identity when compared to SEQ ID NO:17, 19, 21, 23, 25, 36, 50, 66, 68, 70, 72, 74, 76, 78, 80, 82, 84 or 86, or (b) a full complement of the nucleotide sequence, wherein the full complement and the nucleotide sequence consist of the same number of nucleotides and are 100% complementary. The polypeptide may comprise the amino acid sequence of SEQ ID NO:18, 20, 22, 24, 26, 30, 31, 35, 37, 38, 39, 41-49, 51-65, 69, 71, 73, 75, 77, 79, 81, 83, 85, 87, 88-101 or 102. The nucleotide sequence may comprise the nucleotide sequence of SEQ ID NO:17, 19, 21, 23, 25, 36, 50, 66, 68, 70, 72, 74, 76, 78, 80, 82, 84 or 86.

In another embodiment, the present invention concerns a recombinant DNA construct comprising any of the isolated polynucleotides of the present invention operably linked to at least one regulatory sequence, and a cell, a plant, and a seed comprising the recombinant DNA construct. The cell may be eukaryotic, e.g., a yeast, insect or plant cell, or prokaryotic, e.g., a bacterial cell.

### BRIEF DESCRIPTION OF THE DRAWINGS AND SEQUENCE LISTING

The invention can be more fully understood from the following detailed description and the accompanying drawings and Sequence Listing which form a part of this application.

FIG. 1 shows a schematic of the pHSbarENDs2 activation tagging construct (SEQ ID NO:1) used to make the Arabidopsis populations.

FIG. 2 shows a map of the vector pDONR™/Zeo (SEQ ID NO:2). The attP1 site is at nucleotides 570-801; the attP2 site is at nucleotides 2754-2985 (complementary strand).

FIG. 3 shows a map of the vector pDONR™221 (SEQ ID NO:3). The attP1 site is at nucleotides 570-801; the attP2 site is at nucleotides 2754-2985 (complementary strand).

FIG. 4 shows a map of the vector pBC-yellow (SEQ ID NO:4), a destination vector for use in construction of expression vectors for Arabidopsis. The attR1 site is at nucleotides 11276-11399 (complementary strand); the attR2 site is at nucleotides 9695-9819 (complementary strand).

FIG. 5 shows a map of PHP27840 (SEQ ID NO:5), a destination vector for use in construction of expression vectors for soybean. The attR1 site is at nucleotides 7310-7434; the attR2 site is at nucleotides 8890-9014.

FIG. 6 shows a map of PHP23236 (SEQ ID NO:6), a destination vector for use in construction of expression vectors for Gaspe Flint derived maize lines. The attR1 site is at nucleotides 2006-2130; the attR2 site is at nucleotides 2899-3023.

FIG. 7 shows a map of PHP10523 (SEQ ID NO:7), a plasmid DNA present in Agrobacterium strain LBA4404 (Komari et al., Plant J. 10:165-174 (1996); NCBI General Identifier No. 59797027).

FIG. 8 shows a map of PHP23235 (SEQ ID NO:8), a vector used to construct the destination vector PHP23236.

FIG. 9 shows a map of PHP28647 (SEQ ID NO:9), a destination vector for use with maize inbred-derived lines. The attR1 site is at nucleotides 2289-2413; the attR2 site is at nucleotides 3869-3993.

FIG. 10 shows a map of PHP29634 (also called DV11), a destination vector for use with Gaspe Flint derived maize lines.

FIGS. 11A-11F show the multiple alignment of the amino acid sequences of the MATE-efflux polypeptides of SEQ ID NOs: 18, 20, 22, 24, 26, 37, 38, 51, 67, 69, 71, 73, 75, 77, 79, 81, 83, 85 and 87. A majority consensus sequence is presented above the aligned amino acid sequences. Residues that are identical to the residues of SEQ ID NO:18 at a given position are enclosed in a box.

FIG. 12 shows the percent sequence identity and the divergence values for each pair of amino acids sequences of MATE-efflux polypeptides displayed in FIGS. 11A-11F.

FIG. 13 shows the treatment schedule for screening plants with enhanced drought tolerance.

FIGS. 14A and 14B show the % germination curve for wt and At2g04090 transgenic Arabidopsis line, at different quad concentrations.

FIGS. 15A and 15B show the % germination, % greenness and % leaf emergence graph respectively for At2g04090 transgenic line and wt Arabidopsis plants at different quad concentrations.

FIG. 16 shows the comparative graph for wt and At2g04090 transgenic Arabidopsis line for the parameters % germination, % greenness and % leaf emergence, at 60% quad.

FIGS. 17A and 17B show the data for 48 hours % germination for At2g04090 transgenic line ID 25 and a wild-type Arabidopsis line; the experiment was done in triplicate. The results are presented as both a bar graph (FIG. 17A) and a line graph (FIG. 17B).

FIG. 18 shows the ASI, plant height and ear height data for Zm-MATE-EP3 transgenic maize line.

SEQ ID NO:1 is the nucleotide sequence of the pHSbarENDs2 activation tagging vector.

SEQ ID NO:2 is the nucleotide sequence of the GATEWAY® donor vector pDONR™/Zeo.

SEQ ID NO:3 is the nucleotide sequence of the GATEWAY® donor vector pDONR™221.

SEQ ID NO:4 is the nucleotide sequence of pBC-yellow, a destination vector for use with Arabidopsis.

SEQ ID NO:5 is the nucleotide sequence of PHP27840, a destination vector for use with soybean.

SEQ ID NO:6 is the nucleotide sequence of PHP23236, a destination vector for use with Gaspe Flint derived maize lines.

SEQ ID NO:7 is the nucleotide sequence of PHP10523 (Komari et al., Plant J. 10:165-174 (1996); NCBI General Identifier No. 59797027).

SEQ ID NO:8 is the nucleotide sequence of PHP23235, a destination vector for use with Gaspe Flint derived lines.

SEQ ID NO:9 is the nucleotide sequence of PHP28647, a destination vector for use with maize inbred-derived lines.

SEQ ID NO:10 is the nucleotide sequence of the attB1 site.

SEQ ID NO:11 is the nucleotide sequence of the attB2 site.

SEQ ID NO:12 is the nucleotide sequence of the At2g04090-5′attB forward primer, containing the attB1 sequence, used to amplify the At2g04090 protein-coding region.

SEQ ID NO:13 is the nucleotide sequence of the At2g04090-3′attB reverse primer, containing the attB2 sequence, used to amplify the At2g04090 protein-coding region.

SEQ ID NO:14 is the nucleotide sequence of the VC062 primer, containing the T3 promoter and attB1 site, useful to amplify cDNA inserts cloned into a BLUESCRIPT® II SK(+) vector (Stratagene).

SEQ ID NO:15 is the nucleotide sequence of the VC063 primer, containing the T7 promoter and attB2 site, useful to amplify cDNA inserts cloned into a BLUESCRIPT® II SK(+) vector (Stratagene).

SEQ ID NO:16 is the nucleotide sequence of PHP29634 (also called DV11), a destination vector for use with Gaspe Flint derived maize lines.

SEQ ID NO:17 corresponds to NCBI GI No. 18395670, which is the nucleotide sequence from locus At2g04090.

SEQ ID NO:18 corresponds to the amino acid sequence of At2g04090 encoded by SEQ ID NO:17, and corresponds to NCBI GI NO. 15228085.

Table 1 presents SEQ ID NOs for the nucleotide sequences obtained from cDNA clones from maize. The SEQ ID NOs for the corresponding amino acid sequences encoded by the cDNAs are also presented.

TABLE 1
cDNAs Encoding MATE-Efflux Polypeptides

| | | SEQ ID NO: | SEQ ID NO: |
|---|---|---|---|
| Plant | Clone Designation* | (Nucleotide) | (Amino Acid) |
| Corn | cfp6n.pk010.h3 (FIS) | 19 | 20 |
| Corn | cfp1n.pk004.c4 (FIS) | 21 | 22 |
| Corn | cfp6n.pk009.n19 (FIS) | 23 | 24 |
| Corn | cfp5n.pk002.e2 (FIS) | 25 | 26 |
| wheat | wlp1c.pk006.j5 | 66 | 67 |
| Resurrection | En_NODE_45314 | 68 | 69 |
| grass | | | |
| Resurrection | En_NODE_19917 | 70 | 71 |
| grass | | | |
| Resurrection | En_NODE_1677 | 72 | 73 |
| grass | | | |
| Bahia grass | Pn_NODE_53729 | 74 | 75 |
| Bahia grass | Pn_NODE_31640 | 76 | 77 |
| Bahia grass | Pn_NODE_155338 | 78 | 79 |
| Bahia grass | Pn_NODE_21180 | 80 | 81 |
| Bahia grass | Pn_NODE_39122 | 82 | 83 |
| Bahia grass | Pn_NODE_200639 | 84 | 85 |

*Sequence of an entire cDNA insert is the “Full-Insert Sequence” (“FIS”).

SEQ ID NO:27 is the amino acid sequence presented in SEQ ID NO:30086 of U.S. Pat. No. 7,569,389.

SEQ ID NO:28 is the sequence corresponding to NCBI GI NO. 195650919 (Zea mays).

SEQ ID NO.29 is the amino acid sequence presented in SEQ ID NO:8539 of U.S. Pat. No. 7,569,389.

SEQ ID NO:30 is the amino acid sequence corresponding to NCBI GI NO. 242041995 (Sorghum bicolor).

SEQ ID NO:31 is the amino acid sequence presented in SEQ ID NO:17653 of Publication No. US20090070897.

SEQ ID NO.32 is the amino acid sequence corresponding to NCBI GI No. 195619754 (Zea mays).

SEQ ID NO:33 is the amino acid sequence presented in SEQ ID NO:8873 of U.S. Pat. No. 7,569,389.

SEQ ID NO:34 is the amino acid sequence corresponding to NCBI GI No. 223949561 (Zea mays).

SEQ ID NO:35 is the amino acid sequence presented in SEQ ID NO:93375 of Publication No. WO2008034648

SEQ ID NO:36 is the nucleic acid sequence corresponding to a predicted CDS from BAC ZMMBBc0262P05 (AC187156) (Zea mays).

SEQ ID NO:37 is the amino acid sequence of a predicted protein from BAC ZMMBBc0262P05, and is the amino acid sequence encoded by SEQ ID NO:36 (Zea mays).

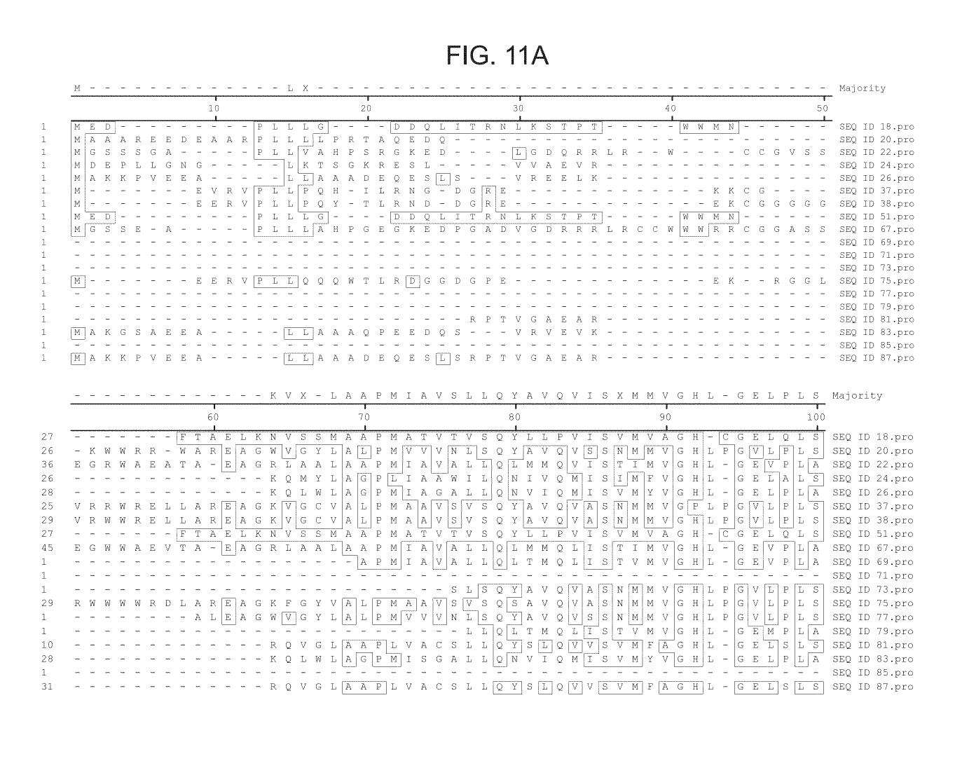

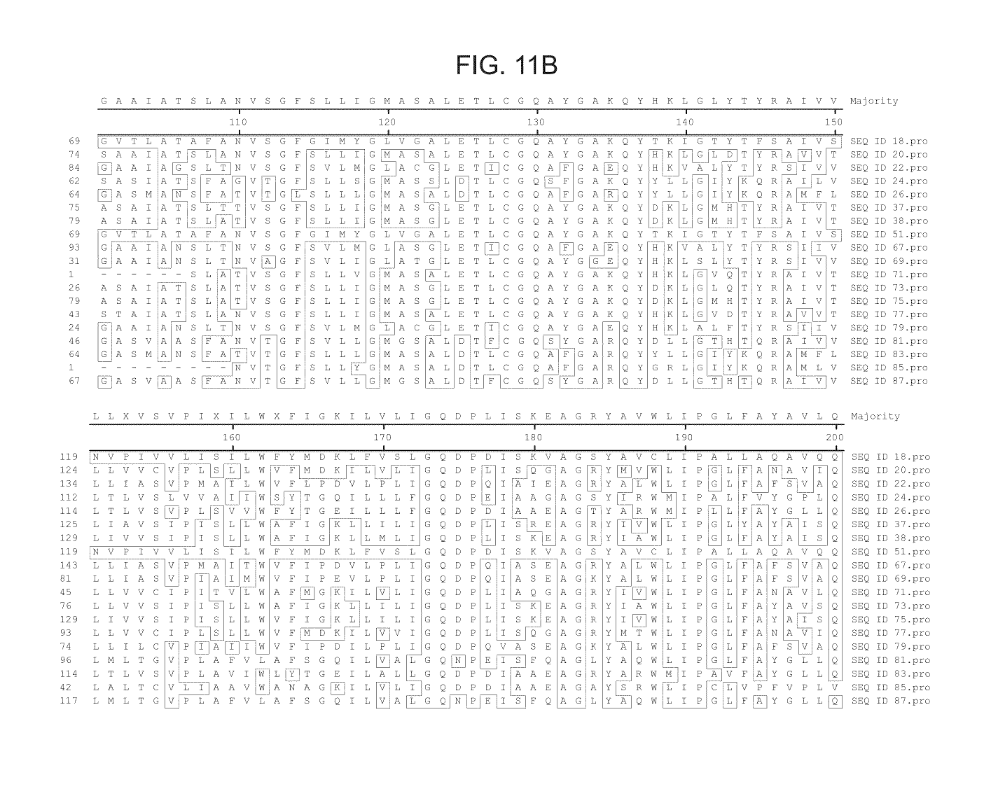

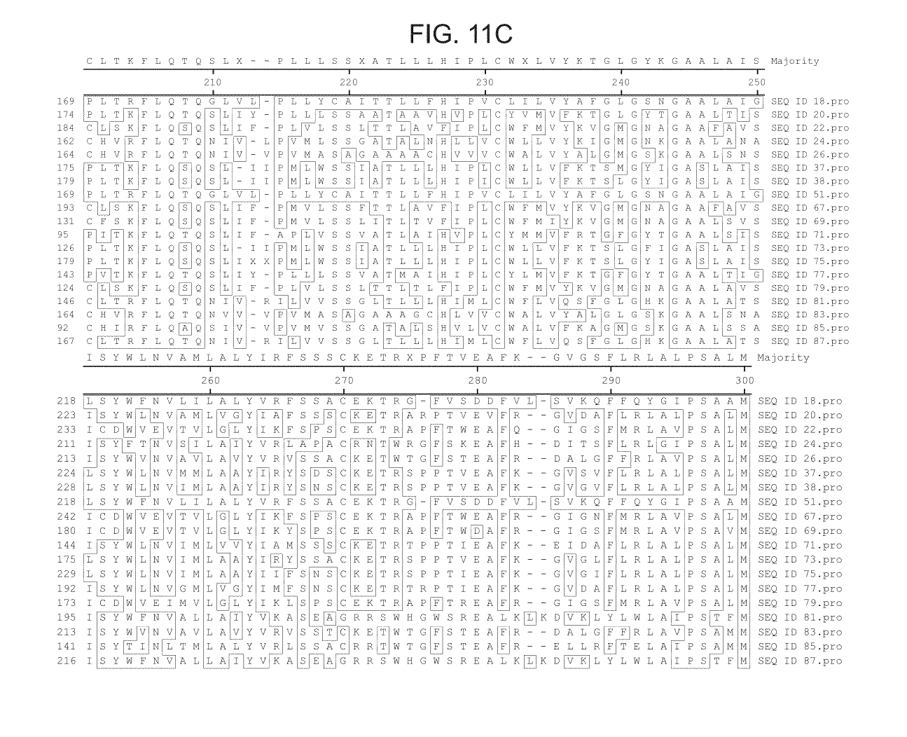

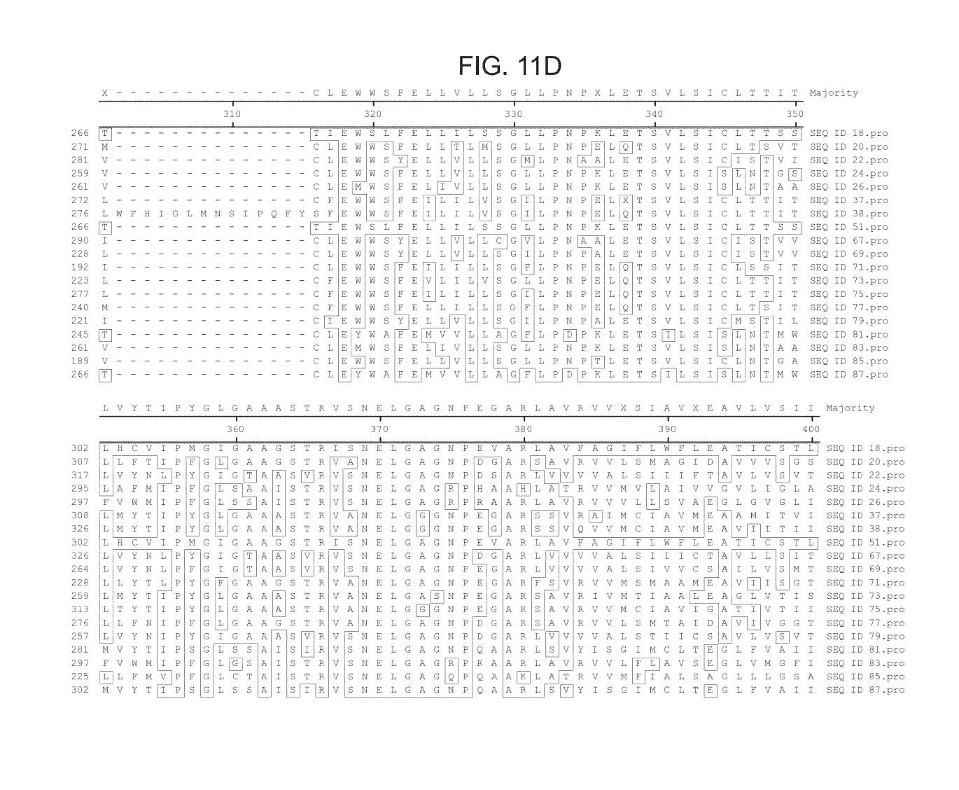

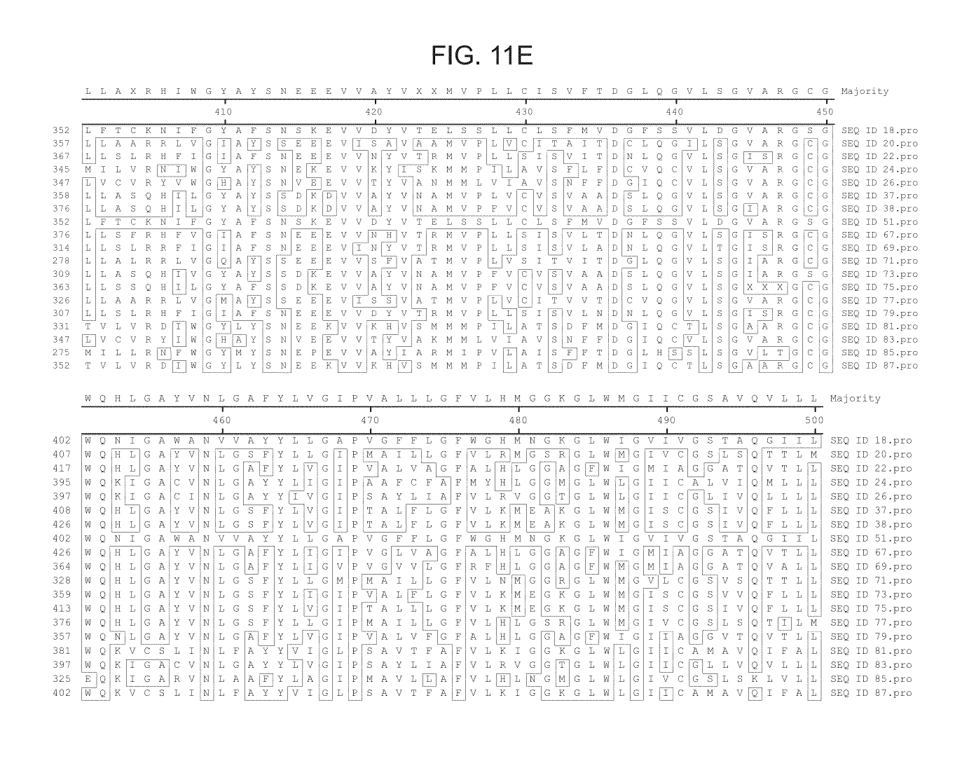

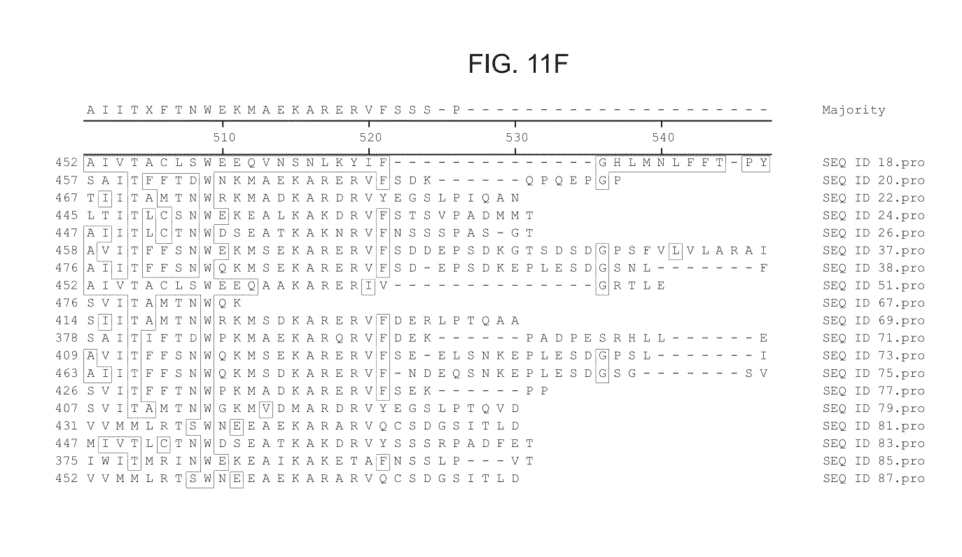

SEQ ID NO.38 is the amino acid sequence corresponding to NCBI GI No. 242088755 (Sorghum bicolor). Based on the sequence alignment of FIG. 11A-11F, this amino acid sequence may have an unspliced intron corresponding to amino acids 277-290.

SEQ ID NO:39 is the amino acid sequence presented in SEQ ID NO:32358 of Patent No. US20060107345 (Triticum aestivum).

SEQ ID NO:40 corresponds to the amino acid sequence of the protein encoded by the gene At2g04100 and corresponds to NCBI GI NO. 22325453 (Arabidopsis thaliana).

SEQ ID NO:41 corresponds to the amino acid sequence of the protein encoded by the gene At2g04050 and corresponds to NCBI GI NO. 15228073 (Arabidopsis thaliana).

SEQ ID NO:42 corresponds to the amino acid sequence of the protein encoded by the gene At2g04070 and corresponds to NCBI GI NO. 186499234 (Arabidopsis thaliana).

SEQ ID NO:43 corresponds to the amino acid sequence of the protein encoded by the gene At2g04080 and corresponds to NCBI GI NO. 30678096 (Arabidopsis thaliana).

SEQ ID NO:44 corresponds to the amino acid sequence of the protein encoded by the gene At2g04040 and corresponds to NCBI GI NO. 15228071 (Arabidopsis thaliana).

SEQ ID NO:45 corresponds to the amino acid sequence of the protein encoded by the gene At1g71140 and corresponds to NCBI GI NO. 30678096 (Arabidopsis thaliana).

SEQ ID NO:46 corresponds to the amino acid sequence of the protein encoded by the gene At1g15170 and corresponds to NCBI GI NO. 15218070 (Arabidopsis thaliana).

SEQ ID NO:47 corresponds to the amino acid sequence of the protein encoded by the gene At1g15180 and corresponds to NCBI GI NO. 18394206 (Arabidopsis thaliana).

SEQ ID NO:48 corresponds to the amino acid sequence of the protein encoded by the gene At1g15160 and corresponds to NCBI GI NO. 15218068 (Arabidopsis thaliana).

SEQ ID NO:49 corresponds to the amino acid sequence of the protein encoded by the gene At1g15150 and corresponds to NCBI GI NO. 22329577 (Arabidopsis thaliana).

SEQ ID NO:50 corresponds to the nucleotide sequence of NCBI GI NO. 334184133, and corresponds to an updated sequence of the At-MATE-EP gene, at locus At2g04090 (Arabidopsis thaliana).

SEQ ID NO:51 corresponds to the amino acid sequence of NCBI GI NO. 334184134, and corresponds to an updated sequence of the At-MATE-EP protein, encoded by the nucleotide sequence given in SEQ ID NO:50 (Arabidopsis thaliana).

SEQ ID NO:52 is the amino acid sequence corresponding to Glyma10g41360, a soybean (Glycine max) predicted protein from predicted coding sequences from Soybean JGI Glyma1.01 genomic sequence from the US Department of energy Joint Genome Institute.

SEQ ID NO:53 is the amino acid sequence corresponding to Glyma06g10850.1, a soybean (Glycine max) predicted protein from predicted coding sequences from Soybean JGI Glyma1.01 genomic sequence from the US Department of energy Joint Genome Institute.

SEQ ID NO:54 is the amino acid sequence corresponding to Glyma10g41340, a soybean (Glycine max) predicted protein from predicted coding sequences from Soybean JGI Glyma1.01 genomic sequence from the US Department of energy Joint Genome Institute.

SEQ ID NO:55 is the amino acid sequence corresponding to Glyma20g25880, a soybean (Glycine max) predicted protein from predicted coding sequences from Soybean JGI Glyma1.01 genomic sequence from the US Department of energy Joint Genome Institute.

SEQ ID NO:56 is the amino acid sequence corresponding to Glyma18g53030, a soybean (Glycine max) predicted protein from predicted coding sequences from Soybean JGI Glyma1.01 genomic sequence from the US Department of energy Joint Genome Institute.

SEQ ID NO:57 is the amino acid sequence corresponding to Glyma10g41370, a soybean (Glycine max) predicted protein from predicted coding sequences from Soybean JGI Glyma1.01 genomic sequence from the US Department of energy Joint Genome Institute.

SEQ ID NO:58 is the amino acid sequence corresponding to Glyma06g47660, a soybean (Glycine max) predicted protein from predicted coding sequences from Soybean JGI Glyma1.01 genomic sequence from the US Department of energy Joint Genome Institute.

SEQ ID NO:59 is the amino acid sequence corresponding to the locus LOC_Os05g48040, a rice (japonica) predicted protein from the Michigan State University Rice Genome Annotation Project Osa1 release 6 (January 2009).

SEQ ID NO:60 is the amino acid sequence corresponding to the locus LOC_Os01g49120, a rice (japonica) predicted protein from the Michigan State University Rice Genome Annotation Project Osa1 release 6 (January 2009).

SEQ ID NO:61 is the amino acid sequence corresponding to the locus LOC_Os01g39180, a rice (japonica) predicted protein from the Michigan State University Rice Genome Annotation Project Osa1 release 6 (January 2009).

SEQ ID NO:62 is the amino acid sequence corresponding to NCBI GI No. 242058365 (Sorghum bicolor).

SEQ ID NO:63 is the amino acid sequence corresponding to NCBI GI No. 242088755 (Sorghum bicolor).

SEQ ID NO:64 is the amino acid sequence corresponding to NCBI GI No. 242041995 (Sorghum bicolor).

SEQ ID NO:65 is the amino acid sequence corresponding to NCBI GI No. 326518786 (Hordeum vulgare).

SEQ ID NO:86 is the nucleotide sequence of Pn_NODE_{—}21180 completed at the N-terminus end using cfp5n.pk002.e2 nucleotide sequence.

SEQ ID NO:87 is the amino acid sequence encoded by SEQ ID NO:86.

SEQ ID NO:88 is the amino acid sequence given in SEQ ID NO:11204 of US publication no. US2011016514 (Panicum virgatum).

SEQ ID NO:89 is the amino acid sequence presented in SEQ ID NO:54943 of US publication no, US20060123505 (Oryza sativa).

SEQ ID NO:90 is the amino acid sequence presented in NCBI GI no. 56784891 (Oryza sativa).

SEQ ID NO:91 is the amino acid sequence presented in SEQ ID NO:52182 of US publication no, US20060123503 (Oryza sativa).

SEQ ID NO:92 is the amino acid sequence presented in NCBI GI no. 215707242 (Oryza sativa).

SEQ ID NO:93 is the amino acid sequence presented in SEQ ID NO:29593 of US publication no. US20110167514 (Panicum virgatum).

SEQ ID NO:94 is the amino acid sequence presented in NCBI GI no. 215740571 (Oryza sativa).

SEQ ID NO:95 is the amino acid sequence presented in SEQ ID NO:238224 of US publication no, US20110214206 (Zea mays).

SEQ ID NO:96 is the amino acid sequence presented in NCBI GI no. 194701508 (Zea mays).

SEQ ID NO:97 is the amino acid sequence presented in SEQ ID NO:155433 of US publication no. US20110131679 (Oryza sativa).

SEQ ID NO:98 is the amino acid sequence presented in NCBI GI no. 194689564 (Zea mays).

SEQ ID NO:99 is the amino acid sequence presented in SEQ ID NO:29593 of US publication no. US20100083407 (Zea mays).

SEQ ID NO:100 is the amino acid sequence presented in SEQ ID NO:205649 of US publication no. US20110214206 (Zea mays).

SEQ ID NO:101 is the amino acid sequence presented in NCBI GI no. 195613120 (Zea mays).

SEQ ID NO:102 is the amino acid sequence presented in SEQ ID NO:26320 of US publication no, US20100083407 (Zea mays).

SEQ ID NO:103 corresponds to TAR Accession No. 6530301899, which is the nucleotide sequence for the genomic DNA of the Arabidopsis thaliana gene At2g04090 (AT-MATE-EP).

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino adds as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

“AT-MATE-Efflux protein” refers to an Arabidopsis thaliana protein encoded by the Arabidopsis thaliana locus At2g04090. The terms “AT-MATE-Efflux protein”, “AT-MATE-Efflux polypeptide” and “AT-MATE-EP” are used interchangeably herein. The protein encoded by the gene At2g04090 (NP_{—}178498; NCBI GI No. 334184134, which replaced the older version of NCBI GI No. 15228085) is a member of the large and ubiquitous multidrug and toxin extrusion family (Hvorup, R. N. et al (2003) Eur. J. Biochem. 270, 799-813).

The term “MATE” stands for “Microbial and Toxic compound Extrusion”, or “multi-antimicrobial extrusion protein”; these terms are used interchangeably herein.

The terms “MATE-Efflux protein”, “MATE-Efflux polypeptide” and “MATE-EP” are used interchangeably herein and refer to homologs of AT-MATE-EP.

Toxins and secondary metabolites are removed from the plant cytoplasm and stored in the vacuole or the cell wall. The compounds that need to be sequestered can be produced endogenously, such as flavonoids, or could be xenobiotics. MATE proteins are a recently identified family of multidrug transporters and are secondary transport proteins with twelve predicted transmembrane domains. Members of this family have been found in all kingdoms of living organisms. There are 58 family members known in Arabidopsis, based on sequence homology (Omote et al. (2006) Trends Pharmaceutical Sci. 27(11): 587-593). The plant MATE proteins characterized so far have been found to be involved in the detoxification of endogenous secondary metabolites and xenobiotics (Brown et al. (1999) Molecular microbiology 31(1):393-395, Eckardt NA (2001) Plant Cell 13:1477-1480).

ALF5, EDS5 and TRANSPARENT TESTA 12 (Tt12) encode Arabidopsis MATE proteins (Ornate at al (2006) Trends Pharmaceutical Sci. 27(11): 587-593; Nawrath et al. (2002) Plant Cell 14: (275-286); Diener et al. (2001) Plant cell 13:1625-1637).

The terms “monocot” and “monocotyledonous plant” are used interchangeably herein. A monocot of the current invention includes the Gramineae.

The terms “dicot” and “dicotyledonous plant” are used interchangeably herein. A dicot of the current invention includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

The growth and emergence of maize silks has a considerable importance in the determination of yield under drought (Fuad-Hassan et al. 2008 Plant Cell Environ. 31:1349-1360). When soil water deficit occurs before flowering, silk emergence out of the husks is delayed while anthesis is largely unaffected, resulting in an increased anthesis-silking interval (ASI) (Edmeades et al. 2000 Physiology and Modeling Kernel set in Maize (eds M. E. Westgate & K. Boote; CSSA (Crop Science Society of America)Special Publication No. 29. Madison, Wis.: CSSA, 43-73). Selection for reduced ASI has been used successfully to increase drought tolerance of maize (Edmeades et al. 1993 Crop Science 33: 1029-1035; Bolanos & Edmeades 1996 Field Crops Research 48:65-80; Bruce et al. 2002 J. Exp. Botany 53:13-25).

Terms used herein to describe thermal time include “growing degree days” (GDD), “growing degree units” (GDU) and “heat units” (HU).

As used herein, the terms “stress tolerant”, “stress resistant”, “tolerant” or “resistant” are used interchangeably herein, and refer to a plant, that, when exposed to a stress condition, shows less of an effect, or no effect, in response to the condition as compared to a corresponding control (or reference) plant, wherein the control plant is exposed to the same stress condition as the test plant.

The terms “stress tolerance” or “stress resistance” as used herein refers to a measure of a plants ability to grow under stress conditions that would detrimentally affect the growth, vigor, yield, and size, of a “non-tolerant” plant of the same species. Stress tolerant plants grow better under conditions of stress than non-stress tolerant plants of the same species. For example, a plant with increased growth rate, compared to a plant of the same species and/or variety, when subjected to stress conditions that detrimentally affect the growth of another plant of the same species would be said to be stress tolerant. A plant with “increased stress tolerance” can exhibit increased tolerance to one or more different stress conditions.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions. Typically, when a transgenic plant comprising a recombinant DNA construct or suppression DNA construct in its genome exhibits increased stress tolerance relative to a reference or control plant, the reference or control plant does not comprise in its genome the recombinant DNA construct or suppression DNA construct.

“Stress tolerance activity” of a polypeptide indicates that over-expression of the polypeptide in a transgenic plant confers increased stress tolerance to the transgenic plant relative to a reference or control plant. For examples, a polypeptide with “osmotic stress tolerance activity” indicates that over-expression of the polypeptide in a transgenic plant confers increased osmotic stress tolerance to the transgenic plant relative to a reference or control plant.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention). “Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino add polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein refers to a post-translationally processed polypeptide; i.e., one from which any pre- or pro-peptides present in the primary translation product have been removed.

“Precursor” protein refers to the primary product of translation of mRNA: i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

A “chloroplast transit peptide” is an amino acid sequence which is translated in conjunction with a protein and directs the protein to the chloroplast or other plastid types present in the cell in which the protein is made (Lee et al. (2008) Plant Cell 20:1603-1622). The terms “chloroplast transit peptide” and “plastid transit peptide” are used interchangeably herein. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632). A “mitochondrial signal peptide” is an amino acid sequence which directs a precursor protein into the mitochondria (Zhang and Glaser (2002) Trends Plant Sci 7:14-21).

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press Cold Spring Harbor, 1989 (hereinafter “Sambrook”).

Turning now to the embodiments:

Embodiments include isolated polynucleotides and polypeptides, recombinant DNA constructs useful for conferring drought tolerance, compositions (such as plants or seeds) comprising these recombinant DNA constructs, and methods utilizing these recombinant DNA constructs.

Isolated Polynucleotides and Polypeptides:

The present invention includes the following isolated polynucleotides and polypeptides:

An isolated polynucleotide comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs (including suppression DNA constructs) of the present invention. The polypeptide is preferably a MATE-Efflux polypeptide. The polypeptide preferably has drought tolerance activity.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102. The polypeptide is preferably a MATE-Efflux polypeptide. The polypeptide preferably has drought tolerance activity

An isolated polynucleotide comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:17, 19, 21, 23, 25, 36, 50, 66, 68, 70, 72, 74, 76, 78, 80, 82, 84 or 86; or (ii) a full complement of the nucleic acid sequence of (i). Any of the foregoing isolated polynucleotides may be utilized in any recombinant DNA constructs (including suppression DNA constructs) of the present invention. The isolated polynucleotide preferably encodes a MATE-efflux polypeptide. The MATE-efflux polypeptide preferably has drought tolerance activity.

An isolated polynucleotide comprising a nucleotide sequence, wherein the nucleotide sequence is hybridizable under stringent conditions with a DNA molecule comprising the full complement of SEQ ID NO:17, 19, 21, 23, 25, 36, 50, 66, 68, 70, 72, 74, 76, 78, 80, 82, 84 or 86. The isolated polynucleotide preferably encodes a a MATE-efflux polypeptide. The a MATE-efflux polypeptide preferably has drought tolerance activity.

An isolated polynucleotide comprising a nucleotide sequence, wherein the nucleotide sequence is derived from SEQ ID NO:17, 19, 21, 23, 25, 36, 50, 66, 68, 70, 72, 74, 76, 78, 80, 82, 84 or 86 by alteration of one or more nucleotides by at least one method selected from the group consisting of: deletion, substitution, addition and insertion. The isolated polynucleotide preferably encodes a a MATE-efflux polypeptide. The a MATE-efflux polypeptide preferably has drought tolerance activity.

An isolated polynucleotide comprising a nucleotide sequence, wherein the nucleotide sequence corresponds to an allele of SEQ ID NO:17, 19, 21, 23, 25, 36, 50, 66, 68, 70, 72, 74, 76, 78, 80, 82, 84 or 86.

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current invention may also be a protein which comprises an amino acid sequence comprising deletion, substitution, insertion and/or addition of one or more amino acids in an amino acid sequence presented in SEQ ID NO:17, 19, 21, 23, 25, 36, 50, 66, 68, 70, 72, 74, 76, 78, 80, 82, 84 or 86. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present invention may also be a protein which is encoded by a nucleic acid comprising a nucleotide sequence comprising deletion, substitution, insertion and/or addition of one or more nucleotides in the nucleotide sequence of SEQ ID NO:17, 19, 21, 23, 25, 36, 50, 66, 68, 70, 72, 74, 76, 78, 80, 82, 84 or 86. Nucleotide deletion, substitution, insertion and/or addition may be accomplished by site-directed mutagenesis or other techniques as mentioned above.

The protein of the present invention may also be a protein which is encoded by a nucleic acid comprising a nucleotide sequence hybridizable under stringent conditions with the complementary strand of the nucleotide sequence of SEQ ID NO:17, 19, 21, 23, 25, 36, 50, 66, 68, 70, 72, 74, 76, 78, 80, 82, 84 or 86.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SOS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SOS, 0.5×SSC at 55° C. for 20 minutes and once in 2×SSC at room temperature for 5 minutes.

Recombinant DNA Constructs and Suppression DNA Constructs:

In one aspect, the present invention includes recombinant DNA constructs (including suppression DNA constructs).

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102: or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:17, 19, 21, 23, 25, 36, 50, 66, 68, 70, 72, 74, 76, 78, 80, 82, 84, or 86: or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide encodes a MATE-efflux polypeptide. The MATE-efflux polypeptide preferably has drought tolerance activity. The MATE-efflux polypeptide may be from Arabidopsis thaliana, Zea mays, Glycine max, Glycine tabacina, Glycine soja Glycine tomentella, Oryza sativa, Paspalum notatum, Eragrostis nindensis, Brassica napus, Sorghum bicolor, Saccharum officinarum, or Triticum aestivum.

In another aspect, the present invention includes suppression DNA constructs.

A suppression DNA construct may comprise at least one regulatory sequence (e.g., a promoter functional in a plant) operably linked to (a) all or part of: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a MATE-efflux polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:17, 19, 21, 23, 25, 36, 50, 66, 68, 70, 72, 74, 76, 78, 80, 82, 84 or 86, or (ii) a full complement of the nucleic acid sequence of (c)(i). The suppression DNA construct may comprise a cosuppression construct, antisense construct, viral-suppression construct, hairpin suppression construct, stem-loop suppression construct, double-stranded RNA-producing construct, RNAi construct, or small RNA construct (e.g., an siRNA construct or an miRNA construct).

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stern-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998): and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros. Science 294:862-864 (2001); have et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNA) in animals and posttranscriptional gene silencing (PTGS) in plants, and likely are incorporated into an RNA-induced silencing complex (RISC) that is similar or identical to that seen for RNAi.

Regulatory Sequences:

A recombinant DNA construct (including a suppression DNA construct) of the present invention may comprise at least one regulatory sequence.

A regulatory sequence may be a promoter.

A number of promoters can be used in recombinant DNA constructs of the present invention. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet, 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the invention, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present invention which causes the desired temporal and spatial expression.

Promoters which are seed or embryo-specific and may be useful in the invention include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant, Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lean and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use in the current invention include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995): NCBI GenBank Accession No, X80206)). Zag2 transcripts can be detected 5 days prior to pollination to 7 to 8 days after pollination (“DAP”), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Additional promoters for regulating the expression of the nucleotide sequences of the present invention in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

Promoters for use in the current invention may include: RIP2, mLIP15, ZmCOR1, Rab17, CaMV 35S, RD29A, B22E, Zag2, SAM synthetase, ubiquitin, CaMV 195, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 200610156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP247 promoter (NCBI accession number: U38790; GI No. 1063664),

Recombinant DNA constructs of the present invention may also include other regulatory sequences, including but not limited to, translation leader sequences, introns, and polyadenylation recognition sequences. In another embodiment of the present invention, a recombinant DNA construct of the present invention further comprises an enhancer or silencer.

An intron sequence can be added to the 5′ untranslated region, the protein-coding region or the 3′ untranslated region to increase the amount of the mature message that accumulates in the cylosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

Any plant can be selected for the identification of regulatory sequences and MATE-efflux polypeptide genes to be used in recombinant DNA constructs of the present invention. Examples of suitable plant targets for the isolation of genes and regulatory sequences would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassaya, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini.

Compositions:

A composition of the present invention includes a transgenic microorganism, cell, plant, and seed comprising the recombinant DNA construct. The cell may be eukaryotic, e.g., a yeast, insect or plant cell, or prokaryotic, e.g., a bacterial cell.

A composition of the present invention is a plant comprising in its genome any of the recombinant DNA constructs (including any of the suppression DNA constructs) of the present invention (such as any of the constructs discussed above). Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristic (e.g., an increased agronomic characteristic optionally under water limiting conditions), or used in a breeding program to produce hybrid seed, which can be grown to produce plants that would exhibit such an altered agronomic characteristic. The seeds may be maize seeds.

The plant may be a monocotyledonous or dicotyledonous plant, for example, a maize or soybean plant, such as a maize hybrid plant or a maize inbred plant. The plant may also be sunflower, sorghum, canola, wheat, alfalfa, cotton, rice, barley, millet, sugar cane or switchgrass.

The recombinant DNA construct may be stably integrated into the genome of the plant.

Particular embodiments include but are not limited to the following:

1. A plant (for example, a maize or soybean plant) comprising in its genome a recombinant DNA construct comprising a polynucleotide operably linked to at least one regulatory sequence, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, and wherein said plant exhibits increased drought tolerance when compared to a control plant not comprising said recombinant DNA construct. The plant may further exhibit an alteration of at least one agronomic characteristic when compared to the control plant.

2. A plant (for example, a maize or soybean plant) comprising in its genome a recombinant DNA construct comprising a polynucleotide operably linked to at least one regulatory sequence, wherein said polynucleotide encodes a MATE-efflux polypeptide, and wherein said plant exhibits increased drought tolerance when compared to a control plant not comprising said recombinant DNA construct. The plant may further exhibit an alteration of at least one agronomic characteristic when compared to the control plant.

3. A plant (for example, a maize or soybean plant) comprising in its genome a recombinant DNA construct comprising a polynucleotide operably linked to at least one regulatory sequence, wherein said polynucleotide encodes a MATE-efflux polypeptide, and wherein said plant exhibits an alteration of at least one agronomic characteristic when compared to a control plant not comprising said recombinant DNA construct.

4. A plant (for example, a maize, rice or soybean plant) comprising in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:17, 19, 21, 23, 25, 36, 50, 66, 68, 70, 72, 74, 76, 78, 80, 82, 84 or 86; or (b) derived from SEQ ID NO:17, 19, 21, 23, 25, 36, 50, 66, 68, 70, 72, 74, 76, 78, 80, 82, 84 or 86 by alteration of one or more nucleotides by at least one method selected from the group consisting of: deletion, substitution, addition and insertion; and wherein said plant exhibits increased tolerance to drought stress, when compared to a control plant not comprising said recombinant DNA construct. The plant may further exhibit an alteration of at least one agronomic characteristic when compared to the control plant.

5. A plant (for example, a maize or soybean plant) comprising in its genome a recombinant DNA construct comprising a polynucleotide operably linked to at least one regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102 and wherein said plant exhibits an alteration of at least one agronomic characteristic when compared to a control plant not comprising said recombinant DNA construct.

6. A plant (for example, a maize, rice or soybean plant) comprising in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:17, 19, 21, 23, 25, 36, 50, 66, 68, 70, 72, 74, 76, 78, 80, 82, 84 or 86; or (b) derived from SEQ ID NO:17, 19, 21, 23, 25, 36, 50, 66, 68, 70, 72, 74, 76, 78, 80, 82, 84 or 86 by alteration of one or more nucleotides by at least one method selected from the group consisting of: deletion, substitution, addition and insertion; and wherein said plant exhibits an alteration of at least one agronomic characteristic when compared to a control plant not comprising said recombinant DNA construct.

7. A plant (for example, a maize or soybean plant) comprising in its genome a suppression DNA construct comprising at least one regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a MATE-efflux polypeptide, and wherein said plant exhibits an alteration of at least one agronomic characteristic when compared to a control plant not comprising said suppression DNA construct.

8. A plant (for example, a maize or soybean plant) comprising in its genome a suppression DNA construct comprising at least one regulatory element operably linked to all or part of (a) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, or (b) a full complement of the nucleic acid sequence of (a), and wherein said plant exhibits an alteration of at least one agronomic characteristic when compared to a control plant not comprising said suppression DNA construct.

9. Any progeny of the above plants in embodiments 1-6, any seeds of the above plants in embodiments 1-6, any seeds of progeny of the above plants in embodiments 1-6, and cells from any of the above plants in embodiments 1-6 and progeny thereof.

In any of the foregoing embodiments 1-9 or any other embodiments of the present invention, the MATE-efflux polypeptide may be from Arabidopsis thaliana, Zea mays, Glycine max, Glycine tabacina, Glycine soja Glycine tomentella, Oryza sativa, Brassica napus, Sorghum bicolor, Paspalum notatum, Eragrostis nindensis, Saccharum officinarum, or Triticum aestivum.

In any of the foregoing embodiments 1-9 or any other embodiments of the present invention, the recombinant DNA construct (or suppression DNA construct) may comprise at least a promoter functional in a plant as a regulatory sequence.

In any of the foregoing embodiments 1-9 or any other embodiments of the present invention, the alteration of at least one agronomic characteristic is either an increase or decrease.

In any of the foregoing embodiments 1-9 or any other embodiments of the present invention, the at least one agronomic characteristic may be selected from the group consisting of greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. For example, the alteration of at least one agronomic characteristic may be an increase in yield, greenness or biomass.

In any of the foregoing embodiments 1-9 or any other embodiments of the present invention, the plant may exhibit the alteration of at least one agronomic characteristic when compared, under water limiting conditions, to a control plant not comprising said recombinant DNA construct (or said suppression DNA construct).

In any of the foregoing embodiments 1-9 or any other embodiments of the present invention, the plant may exhibit less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

“Drought” refers to a decrease in water availability to a plant that, especially when prolonged, can cause damage to the plant or prevent its successful growth (e.g., limiting plant growth or seed yield). “Water limiting conditions” refers to a plant growth environment where the amount of water is not sufficient to sustain optimal plant growth and development. The terms “drought” and “water limiting conditions” are used interchangeably herein.

“Drought tolerance” is a trait of a plant to survive under drought conditions over prolonged periods of time without exhibiting substantial physiological or physical deterioration.

“Drought tolerance activity” of a polypeptide indicates that over-expression of the polypeptide in a transgenic plant confers increased drought tolerance to the transgenic plant relative to a reference or control plant.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or suppression DNA construct in its genome exhibits increased drought tolerance relative to a reference or control plant, the reference or control plant does not comprise in its genome the recombinant DNA construct or suppression DNA construct.

The terms “percentage germination” and “percentage seedling emergence” are used interchangeably herein, and refer to the percentage of seeds that germinate, when compared to the total number of seeds being tested.

“Germination” as used herein refers to the emergence of the radicle.

The term “radicle” as used herein refers to the embryonic root of the plant, and is terminal part of embryonic axis. It grows downward in the soil, and is the first part of a seedling to emerge from the seed during the process of germination.

The range of stress and stress response depends on the different plants which are used for the invention, i.e. it varies for example between a plant such as wheat and a plant such as Arabidopsis.

Osmosis is defined as the movement of water from low solute concentration to high solute concentration up a concentration gradient.

“Osmotic pressure” of a solution as defined herein is defined as the pressure exerted by the solute in the system. A solution with higher concentration of solutes would have higher osmotic pressure. All solutes exhibit osmotic pressure. Osmotic pressure increases as concentration of the solute increases.

The osmotic pressure exerted by 250 mM NaCl (sodium chloride) is 1.23 MPa (megapascals) (Werner, J. E. et. al. (1995) Physiologia Plantarum 93: 659-666).

As used herein, the terms “osmotic stress” and “salinity stress” are used interchangeably herein and refer to any stress which is associated with or induced by elevated concentrations of osmolytes and which result in a perturbation in the osmotic potential of the intracellular or extracellular environment of a cell. The term “osmotic stress” as used herein refers to stress exerted when the osmotic potential of the extracellular environment of the cell, tissue, seed, organ or whole plant is increased and the water potential is lowered and a substance that blocks water absorption (osmolyte) is persistently applied to the cell, tissue, seed, organ or whole plant.

With respect to the osmotic stress assay, the term “quad” as used herein refers to four components that impart osmotic stress. A “quad assay” or “quad media”, as used herein, would therefore comprise four components that impart osmotic stress, e.g., sodium chloride, sorbitol, mannitol and PEG.

An increase in the osmotic pressure of the media solution would result in increase in osmotic potential. Examples of conditions that induce osmotic stress include, but are not limited to, salinity, drought, heat, chilling and freezing.

In one embodiment of the invention the osmotic pressure of the media for subjecting the plants to osmotic stress is from 0.4-1.23 MPa. In other embodiments of the invention, the osmotic pressure of the media for subjecting the plants to osmotic stress is 0.4 MPa, 0.5 MPa, 0.6 MPa, 0.7 MPa, 0.8 MPa, 0.9 MPa, 1 MPa, 1.1 MPa, 1.2 MPa or 1.23 MPa. In other embodiments of the invention, the osmotic pressure of the media for subjecting the plants to osmotic stress is at least 0.4 MPa, 0.5 MPa, 0.6 MPa, 0.7 MPa, 0.8 Mpa, 0.9 MPa, 1 MPa, 1.1 MPa, 1.2 MPa or 1.23 MPa. In another embodiment of the invention, the osmotic pressure of the media for subjecting the plants to osmotic stress is 1.23 MPa

The terms “solute” and “osmolytes” are used interchangeably herein and refer to substances that lower the water potential. Examples of such substances include, but are not limited to, ionic osmolytes and nonionic osmolytes.

Ionic solutes can be water soluble inorganic solutes such as sodium chloride (NaCl). Examples of water soluble inorganic solutes include, but are not limited to, NaCl, KCl (potassium chloride), LiCl (lithium chloride), CsCl (cesium chloride), RbCl (Rubidium chloride) and CaCl2 (calcium chloride), sodium sulfate, magnesium sulfate, calcium sulfate, sodium chloride, magnesium chloride, calcium chloride, potassium chloride, etc., salts of agricultural fertilizers and salts associated with alkaline or acid soil conditions (Werner J. E. et al (1995) Physiologia Plantarum 93: 659-666; U.S. Pat. No. 7,253,338).

Examples of non-ionic osmolytes include, but are not limited to, sugars, sugar alcohols, and high molecular weight polymeric osmolytes.

Any sugar alcohol that is mostly metabolically inert can be used as an osmolyte for the methods described in the current invention. Examples of sugar alcohols that can be used as an osmolyte for the methods described in the current invention include, but are not limited to, mannitol, sorbitol, xylitol, lactitol and maltitol. Combination of two or more sugar alcohols may also be used.

Examples of other sugars that can be used as an osmolyte for the methods described in the current invention include, but are not limited to, melibiose and sucrose.

“High-molecular weight polymeric solutes” as used herein refer to polymeric solutes that largely do not permeate into the plant cells. Examples of high-molecular weight polymeric solutes that can be used for lowering the water potential, include, but are not limited to, polyethylene glycol (PEG), polypropylene glycols and dextran (U.S. Pat. No. 5,464,769A; Money N. P., Plant Physiol. (1989) 91:766-769; Lagerwerff, J. V. et al. (1961) Science 133:1486-1487; Heyser, J. E. et al (1981) Plant Physiol. 68:1454-1459). Polyethylene glycol (PEG) is a polymer produced in a range of molecular weights. PEG of molecular weight 6000 or above largely cannot enter the pores of plant cells (Verslues, P. E. et al (2006) Plant Journal 45:523-539; Carpita, N. et al., (1979) Science 205:1144-1147; Oertli, J. J. (1985) J. Plant Physiol. 121:295-300).

PEG of higher molecular weight (>=3000) can be used for the methods described in the current invention. In an embodiment, PEG having a molecular weight between 3000 and 35000 can be used for the methods disclosed in the current invention. In one embodiment. PEG 4000, PEG 6000, PEG 8000 can be used for the methods described in the current invention. In one embodiment, PEG of molecular weight higher than 8000 can be used for the methods described herein.

The terms “tolerant to osmotic stress”, “resistant to osmotic stress” and “osmotically tolerant” are used interchangeably herein, and refer to a plant, that when exposed to an osmotic stress condition, shows less of an effect, or no effect, in response to the condition as compared to a corresponding control (or reference plant), wherein the control plant is exposed to the same osmotic stress condition as the test plant.

A plant identified using the methods disclosed in the current invention exhibits increased tolerance to osmotic stress when grown on a medium which contains a higher content of osmolytes compared to a medium the corresponding reference plant is capable of growing on.

“Triple stress” as used herein refers to the abiotic stress exerted on the plant by the combination of drought stress, high temperature stress and high light stress.

The terms “heat stress” and “temperature stress” are used interchangeably herein, and are defined as where ambient temperatures are hot enough for sufficient time that they cause damage to plant function or development, which might be reversible or irreversible in damage. “High temperature” can be either “high air temperature” or “high soil temperature”, “high day temperature” or “high night temperature, or a combination of more than one of these.

In one embodiment of the invention, the ambient temperature can be in the range of 30° C. to 36° C. In one embodiment of the invention, the duration for the high temperature stress could be in the range of 1-16 hours.

“High light intensity” and “high irradiance” and “light stress” are used interchangeably herein, and refer to the stress exerted by subjecting plants to light intensities that are high enough for sufficient time that they cause photoinhibition damage to the plant.

In one embodiment of the invention, the light intensity can be in the range of 250 μE to 450 μE. In one embodiment of the invention, the duration for the high light intensity stress could be in the range of 12-16 hours.

“Triple stress tolerance” is a trait of a plant to survive under the combined stress conditions of drought, high temperature and high light intensity over prolonged periods of time without exhibiting substantial physiological or physical deterioration.

“Paraquat” is an herbicide that exerts oxidative stress on the plants. Paraquat, a bipyridylium herbicide, acts by intercepting electrons from the electron transport chain at PSI. This reaction results in the production of bipyridyl radicals that readily react with dioxygen thereby producing superoxide. Paraquat tolerance in a plant has been associated with the scavenging capacity for oxyradicals (Lannelli, M. A. et al (1999) J Exp Botany, Vol. 50, No, 333, pp. 523-532). Paraquat resistant plants have been reported to have higher tolerance to other oxidative stresses as well.

“Paraquat stress” is defined as stress exerted on the plants by subjecting them to Paraquat concentrations ranging from 0.03 to 0.3 μM.

Many adverse environmental conditions such as drought, salt stress, and use of herbicide promote the overproduction of reactive oxygen species (ROS) in plant cells. ROS such as singlet oxygen, superoxide radicals, hydrogen peroxide (H₂O₂), and hydroxyl radicals are believed to be the major factor responsible for rapid cellular damage due to their high reactivity with membrane lipids, proteins, and DNA (Miter, R. (2002) Trends Plant Sci Vol. 7 No. 9).

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions. Examples of stress include, but are not limited to sub-optimal conditions associated with salinity, drought, temperature, pathogens, metal, chemical, and oxidative stresses.

“Stress tolerance activity” of a polypeptide indicates that over-expression of the polypeptide in a transgenic plant confers increased stress tolerance to the transgenic plant relative to a reference or control plant. A polypeptide with “triple stress tolerance activity” indicates that over-expression of the polypeptide in a transgenic plant confers increased triple stress tolerance to the transgenic plant relative to a reference or control plant. A polypeptide with “paraquat stress tolerance activity” indicates that over-expression of the polypeptide in a transgenic plant confers increased Paraquat stress tolerance to the transgenic plant relative to a reference or control plant.

Typically, when a transgenic plant comprising a recombinant DNA construct or suppression DNA construct in its genome exhibits increased stress tolerance relative to a reference or control plant, the reference or control plant does not comprise in its genome the recombinant DNA construct or suppression DNA construct.

A plant selected using the methods of the present invention can grow better, can have higher yields and/or can produce more seeds under stress conditions, as compared to a control plant. A plant selected using the methods disclosed in the current invention is capable of substantially normal growth under environmental conditions where the corresponding reference plant shows reduced growth, metabolism or viability, or increased male or female sterility.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days. The following variables may be measured during drought stress and well watered treatments of transgenic plants and relevant control plants:

The variable “% area chg_start chronic−acute2” is a measure of the percent change in total area determined by remote visible spectrum imaging between the first day of chronic stress and the day of the second acute stress.

The variable “% area chg_start chronic−end chronic” is a measure of the percent change in total area determined by remote visible spectrum imaging between the first day of chronic stress and the last day of chronic stress.

The variable “% area chg_start chronic−harvest” is a measure of the percent change in total area determined by remote visible spectrum imaging between the first day of chronic stress and the day of harvest.

The variable “% area chg_start chronic−recovery24 hr” is a measure of the percent change in total area determined by remote visible spectrum imaging between the first day of chronic stress and 24 hrs into the recovery (24 hrs after acute stress 2).

The variable “psii_acute1” is a measure of Photosystem II (PSII) efficiency at the end of the first acute stress period. It provides an estimate of the efficiency at which light is absorbed by PSH antennae and is directly related to carbon dioxide assimilation within the leaf.

The variable “psii_acute2” is a measure of Photosystem II (PSII) efficiency at the end of the second acute stress period. It provides an estimate of the efficiency at which light is absorbed by PSII antennae and is directly related to carbon dioxide assimilation within the leaf.

The variable “fv/fm_acute1” is a measure of the optimum quantum yield (Fv/Fm) at the end of the first acute stress−(variable fluorescence difference between the maximum and minimum fluorescence/maximum fluorescence).

The variable “fv/fm_acute2” is a measure of the optimum quantum yield (Fv/Fm) at the end of the second acute stress−(variable flourescence difference between the maximum and minimum fluorescence/maximum fluorescence).

The variable “leaf rolling harvest” is a measure of the ratio of top image to side image on the day of harvest.

The variable “leaf rolling_recovery24 hr” is a measure of the ratio of top image to side image 24 hours into the recovery.

The variable “Specific Growth Rate (SGR)” represents the change in total plant surface area (as measured by Lemna Tec Instrument) over a single day (Y(t)=Y0*e^{r}*^{t}). Y(t)=Y0*e^{r}*^{t} is equivalent to % change in Y/Δt where the individual terms are as follows: Y(t)=Total surface area at t; Y0=Initial total surface area (estimated): r=Specific Growth Rate day⁻¹, and t=Days After Planting (“DAP”).

The variable “shoot dry weight” is a measure of the shoot weight 96 hours after being placed into a 104° C. oven.

The variable “shoot fresh weight” is a measure of the shoot weight immediately after being cut from the plant.

The Examples below describe some representative protocols and techniques for simulating drought conditions and/or evaluating drought tolerance.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to a control or reference plant).

One of ordinary skill in the art would readily recognize a suitable control OF reference plant to be utilized when assessing or measuring an agronomic characteristic or phenotype of a transgenic plant in any embodiment of the present invention in which a control plant is utilized (e.g., compositions or methods as described herein). For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct) (i.e., the progeny not comprising the recombinant DNA construct (or the suppression DNA construct) is the control or reference plant).

2. Introgression of a recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line i.e., the first hybrid line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Embodiments include:

In one embodiment, a plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, and wherein said plant exhibits either increased drought tolerance, increased osmotic stress tolerance, or both, when compared to a control plant not comprising said recombinant DNA construct.

In another embodiment, a plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, and wherein said plant exhibits an alteration of at least one agronomic characteristic when compared to a control plant not comprising said recombinant DNA construct. Optionally, the plant exhibits said alteration of said at least one agronomic characteristic when compared, under water limiting conditions, to said control plant not comprising said recombinant DNA construct. The at least one agronomic trait may be yield, biomass, or both and the alteration may be an increase.

In one embodiment, a plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, and wherein said plant exhibits increased tolerance to osmotic stress when compared to a control plant not comprising said recombinant DNA construct.

In another embodiment, the present invention includes any of the plants of the present invention wherein the plant is selected from the group consisting of: Arabidopsis, maize, soybean, sunflower, sorghum, canola, wheat, alfalfa, cotton, rice, barley, millet, sugar cane and switchgrass.

In another embodiment, the present invention includes seed of any of the plants of the present invention, wherein said seed comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, and wherein a plant produced from said seed exhibits either an increase in at least one trait selected from the group consisting of: drought tolerance, osmotic stress tolerance, yield and biomass, when compared to a control plant not comprising said recombinant DNA construct.

In another embodiment, a method of increasing drought tolerance in a plant, comprising: (a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) regenerating a transgenic plant from the regenerable plant cell after step (a), wherein the transgenic plant comprises in its genome the recombinant DNA construct; and (c) obtaining a transgenic plant from step (b), or a progeny plant derived from the transgenic plant of step (b), wherein said transgenic plant or progeny plant comprises in its genome the recombinant DNA construct and exhibits increased drought tolerance when compared to a control plant not comprising the recombinant DNA construct.

In another embodiment, a method of increasing osmotic stress tolerance in a plant, comprising; (a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) regenerating a transgenic plant from the regenerable plant cell after step (a), wherein the transgenic plant comprises in its genome the recombinant DNA construct; and (c) obtaining a transgenic plant from step (b), or a progeny plant derived from the transgenic plant of step (b), wherein said transgenic plant or progeny plant comprises in its genome the recombinant DNA construct and exhibits increased osmotic stress tolerance when compared to a control plant not comprising the recombinant DNA construct.

In another embodiment, a method of evaluating drought tolerance in a plant, comprising: (a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) obtaining a transgenic plant from step (b), or a progeny plant derived from the transgenic plant, wherein the transgenic plant or progeny plant comprises in its genome the recombinant DNA construct; and (c) evaluating the progeny plant for drought tolerance compared to a control plant not comprising the recombinant DNA construct.

In another embodiment, a method of increasing abiotic stress tolerance in a plant, comprising: (a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) regenerating a transgenic plant from the regenerable plant cell after step (a), wherein the transgenic plant comprises in its genome the recombinant DNA construct; and (c) obtaining a transgenic plant from step (b), or a progeny plant derived from the transgenic plant of step (b), wherein said transgenic plant or progeny plant comprises in its genome the recombinant DNA construct and exhibits increased tolerance to at least one abiotic stress selected from the group consisting of drought stress, osmotic stress, heat stress, high light stress, salt stress, paraquat stress and cold temperature stress, when compared to a control plant not comprising the recombinant DNA construct.

In another embodiment, a method of determining an alteration of at least one agronomic characteristic in a plant, comprising: (a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, wherein the transgenic plant comprises in its genome the recombinant DNA construct; (c) obtaining a transgenic plant from step (b), or a progeny plant derived from the transgenic plant, wherein the transgenic plant or progeny plant comprises in its genome the recombinant DNA construct; and (d) determining whether the progeny plant exhibits an alteration of at least one agronomic characteristic when compared to a control plant not comprising the recombinant DNA construct. Optionally, said determining step (d) comprises determining whether the transgenic plant exhibits an alteration of at least one agronomic characteristic when compared, under water limiting conditions, to a control plant not comprising the recombinant DNA construct. The at least one agronomic trait may be yield, biomass, or both and the alteration may be an increase.

In another embodiment, the present invention includes any of the methods of the present invention wherein the plant is selected from the group consisting of: Arabidopsis, maize, soybean, sunflower, sorghum, canoe, wheat, alfalfa, cotton, rice, barley, millet, sugar cane and switchgrass.

In another embodiment, the present invention includes an isolated polynucleotide comprising: (a) a nucleotide sequence encoding a polypeptide with drought tolerance activity, wherein the polypeptide has an amino acid sequence of at least 90% sequence identity when compared to SEQ ID NO:17, 19, 21, 23, 25, 36, 50, 66, 68, 70, 72, 74, 76, 78, 80, 82, 84 or 86, or (b) a full complement of the nucleotide sequence, wherein the full complement and the nucleotide sequence consist of the same number of nucleotides and are 100% complementary. The polypeptide may comprise the amino acid sequence of SEQ ID NO:18, 20, 22, 24, 26, 30, 31, 35, 37, 38, 39, 41-49, 51-65, 69, 71, 73, 75, 77, 79, 81, 83, 85, 87, 88-101 or 102. The nucleotide sequence may comprise the nucleotide sequence of SEQ ID NO:17, 19, 21, 23, 25, 36, 50, 66, 68, 70, 72, 74, 76, 78, 80, 82, 84 or 86.

In another embodiment, the present invention concerns a recombinant DNA construct comprising any of the isolated polynucleotides of the present invention operably linked to at least one regulatory sequence, and a cell, a plant, and a seed comprising the recombinant DNA construct. The cell may be eukaryotic, e.g., a yeast, insect or plant cell, or prokaryotic, e.g., a bacterial cell.

In one embodiment, more than one MATE-efflux polypeptide may be overexpressed together in a plant cell. In one embodiment, the polypeptide encoded by the At2g04090 gene may be overexpressed along with another family member of the MATE-efflux proteins in a plant cell. In one embodiment, the polypeptide encoded by At2g04090 gene is overexpressed along with the polypeptide encoded by the At2g4100 gene.

Methods:

Methods include but are not limited to methods for increasing drought tolerance in a plant, methods for evaluating drought tolerance in a plant, methods for altering an agronomic characteristic in a plant, methods for determining an alteration of an agronomic characteristic in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

Methods include but are not limited to the following:

A method for transforming a cell comprising transforming a cell with any of the isolated polynucleotides of the present invention. The cell transformed by this method is also included. In particular embodiments, the cell is eukaryotic cell, e.g., a yeast, insect or plant cell, or prokaryotic, e.g., a bacterial cell.

A method for producing a transgenic plant comprising transforming a plant cell with any of the isolated polynucleotides or recombinant DNA constructs (including suppression DNA constructs) of the present invention and regenerating a transgenic plant from the transformed plant cell. The invention is also directed to the transgenic plant produced by this method, and transgenic seed obtained from this transgenic plant. The transgenic plant obtained by this method may be used in other methods of the present invention.

A method for isolating a polypeptide of the invention from a cell or culture medium of the cell, wherein the cell comprises a recombinant DNA construct comprising a polynucleotide of the invention operably linked to at least one regulatory sequence, and wherein the transformed host cell is grown under conditions that are suitable for expression of the recombinant DNA construct.

A method of altering the level of expression of a polypeptide of the invention in a host cell comprising: (a) transforming a host cell with a recombinant DNA construct of the present invention; and (b) growing the transformed host cell under conditions that are suitable for expression of the recombinant DNA construct wherein expression of the recombinant DNA construct results in production of altered levels of the polypeptide of the invention in the transformed host cell.

A method of increasing drought tolerance in a plant, comprising: (a) introducing into a regenerable plant cell a recombinant DNA construct comprising a polynucleotide operably linked to at least one regulatory sequence (for example, a promoter functional in a plant), wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; and (b) regenerating a transgenic plant from the regenerable plant cell after step (a), wherein the transgenic plant comprises in its genome the recombinant DNA construct and exhibits increased drought tolerance when compared to a control plant not comprising the recombinant DNA construct. The method may further comprise (c) obtaining a progeny plant derived from the transgenic plant, wherein said progeny plant comprises in its genome the recombinant DNA construct and exhibits increased drought tolerance when compared to a control plant not comprising the recombinant DNA construct.

A method of increasing drought tolerance, the method comprising: (a) introducing into a regenerable plant cell a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:17, 19, 21, 23, 25, 36, 50, 66, 68, 70, 72, 74, 76, 78, 80, 82, 84 or 86; or (b) derived from SEQ ID NO:17, 19, 21, 23, 25, 36, 50, 66, 68, 70, 72, 74, 76, 78, 80, 82, 84 or 86 by alteration of one or more nucleotides by at least one method selected from the group consisting of: deletion, substitution, addition and insertion; and (b) regenerating a transgenic plant from the regenerable plant cell after step (a), wherein the transgenic plant comprises in its genome the recombinant DNA construct and exhibits increased drought tolerance when compared to a control plant not comprising the recombinant DNA construct. The method may further comprise (c) obtaining a progeny plant derived from the transgenic plant, wherein said progeny plant comprises in its genome the recombinant DNA construct and exhibits increased drought tolerance, when compared to a control plant not comprising the recombinant DNA construct.

A method of evaluating drought tolerance in a plant, comprising (a) obtaining a transgenic plant, wherein the transgenic plant comprises in its genome a recombinant DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) obtaining a progeny plant derived from said transgenic plant, wherein the progeny plant comprises in its genome the recombinant DNA construct; and (c) evaluating the progeny plant for drought tolerance compared to a control plant not comprising the recombinant DNA construct.

A method of evaluating drought tolerance, the method comprising: (a) obtaining a transgenic plant, wherein the transgenic plant comprises in its genome a recombinant DNA construct comprising a polynucleotide operably linked to at least one regulatory element, wherein said polynucleotide comprises a nucleotide sequence, wherein the nucleotide sequence is; (a) hybridizable under stringent conditions with a DNA molecule comprising the full complement of SEQ ID NO:17, 19, 21, 23, 25, 36, 50, 66, 68, 70, 72, 74, 76, 78, 80, 82, 84 or 86; or (b) derived from SEQ ID NO:17, 19, 21, 23, 25, 36, 50, 66, 68, 70, 72, 74, 76, 78, 80, 82, 84 or 86 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) evaluating the progeny plant for increased drought tolerance, when compared to a control plant not comprising the recombinant DNA construct.

A method of evaluating drought tolerance in a plant, comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102, or (ii) a full complement of the nucleic acid sequence of (a)(i); (b) obtaining a progeny plant derived from said transgenic plant, wherein the progeny plant comprises in its genome the suppression DNA construct; and (c) evaluating the progeny plant for drought tolerance compared to a control plant not comprising the suppression DNA construct.

A method of evaluating drought tolerance in a plant, comprising (a) obtaining a transgenic plant, wherein the transgenic plant comprises in its genome a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a MATE-efflux polypeptide; (b) obtaining a progeny plant derived from the transgenic plant, wherein the progeny plant comprises in its genome the suppression DNA construct; and (c) evaluating the progeny plant for drought tolerance compared to a control plant not comprising the suppression DNA construct.

A method of determining an alteration of an agronomic characteristic in a plant, comprising (a) obtaining a transgenic plant, wherein the transgenic plant comprises in its genome a recombinant DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102; (b) obtaining a progeny plant derived from said transgenic plant, wherein the progeny plant comprises in its genome the recombinant DNA construct; and (c) determining whether the progeny plant exhibits an alteration in at least one agronomic characteristic when compared, optionally under water limiting conditions, to a control plant not comprising the recombinant DNA construct.

A method of determining an alteration of an agronomic characteristic in a plant, comprising (a) obtaining a transgenic plant, wherein the transgenic plant comprises in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:17, 19, 21, 23, 25, 36, 50, 66, 68, 70, 72, 74, 76, 78, 80, 82, 84 or 86; or (b) derived from SEQ ID NO:17, 19, 21, 23, 25, 36, 50, 66, 68, 70, 72, 74, 76, 78, 80, 82, 84 or 86 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) determining whether the progeny plant exhibits an alteration in at least one agronomic characteristic when compared, optionally under water limiting conditions, to a control plant not comprising the recombinant DNA construct. The polynucleotide preferably encodes a MATE-efflux polypeptide. The MATE-efflux polypeptide preferably has drought tolerance activity.

A method of determining an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102 or (ii) a full complement of the nucleic acid sequence of (i); (b) obtaining a progeny plant derived from said transgenic plant, wherein the progeny plant comprises in its genome the suppression DNA construct; and (c) determining whether the progeny plant exhibits an alteration in at least one agronomic characteristic when compared, optionally under water limiting conditions, to a control plant not comprising the suppression DNA construct.

A method of determining an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a MATE-efflux polypeptide; (b) obtaining a progeny plant derived from said transgenic plant, wherein the progeny plant comprises in its genome the suppression DNA construct; and (c) determining whether the progeny plant exhibits an alteration in at least one agronomic characteristic when compared, optionally under water limiting conditions, to a control plant not comprising the suppression DNA construct.

A method of producing seed (for example, seed that can be sold as a drought tolerant product offering) comprising any of the preceding methods, and further comprising obtaining seeds from said progeny plant, wherein said seeds comprise in their genome said recombinant DNA construct (or suppression DNA construct).

In any of the preceding methods or any other embodiments of methods of the present invention, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present invention, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present invention, the at least one agronomic characteristic may be selected from the group consisting of greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. The alteration of at least one agronomic characteristic may be an increase in yield, greenness or biomass.

In any of the preceding methods or any other embodiments of methods of the present invention, the plant may exhibit the alteration of at least one agronomic characteristic when compared, under water limiting conditions, to a control plant not comprising said recombinant DNA construct (or said suppression DNA construct).

In any of the preceding methods or any other embodiments of methods of the present invention, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant invention.

The introduction of recombinant DNA constructs of the present invention into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

### EXAMPLES

The present invention is further illustrated in the following Examples, in which parts and percentages are by weight and degrees are Celsius, unless otherwise stated. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Creation of an Arabidopsis Population with Activation-Tagged Genes

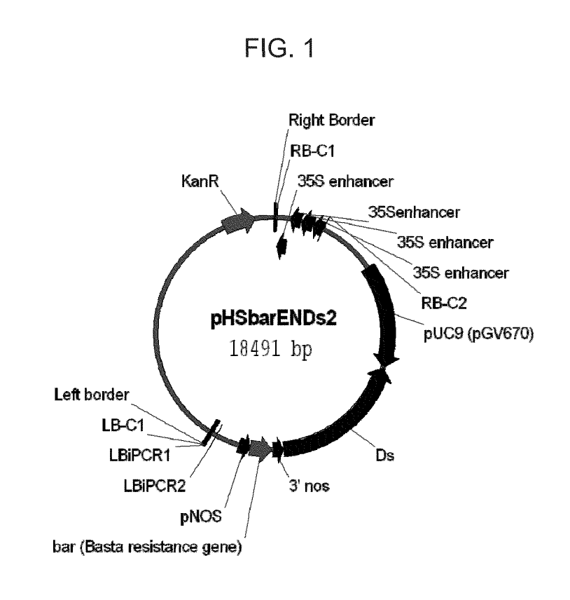

An 18.5-kb T-DNA based binary construct was created, pHSbarENDs2 (FIG. 1; SEQ ID NO:1), that contains four multimerized enhancer elements derived from the Cauliflower Mosaic Virus 35 S promoter (corresponding to sequences −341 to −64, as defined by Odell et al., Nature 313:810-812 (1985)). The construct also contains vector sequences (pUC9) and a polylinker to allow plasmid rescue, transposon sequences (Ds) to remobilize the T-DNA, and the bar gene to allow for glufosinate selection of transgenic plants. In principle, only the 10.8-kb segment from the right border (RB) to left border (LB) inclusive will be transferred into the host plant genome. Since the enhancer elements are located near the RB, they can induce cis-activation of genomic loci following T-DNA integration.

Arabidopsis activation-tagged populations were created by whole plant Agrobacterium transformation. The pHSbarENDs2 construct was transformed into Agrobacterium tumefaciens strain C58, grown in LB at 25° C. to OD600 ˜1.0. Cells were then pelleted by centrifugation and resuspended in an equal volume of 5% sucrose/0.05% Silwet L-77 (OSI Specialties, Inc). At early bolting, soil grown Arabidopsis thaliana ecotype Col-0 were top watered with the Agrobacterium suspension. A week later, the same plants were top watered again with the same Agrobacterium strain in sucrose/Silwet. The plants were then allowed to set seed as normal. The resulting T1 seed were sown on soil, and transgenic seedlings were selected by spraying with glufosinate (Finale); AgrEvo; Bayer Environmental Science). A total of 100,000 glufosinate resistant T1 seedlings were selected. T2 seed from each line was kept separate.

### Example 2

### Screens to Identify Lines with Enhanced Drought Tolerance

Quantitative Drought Screen:

From each of 96,000 separate T1 activation-tagged lines, nine glufosinate resistant T2 plants are sown, each in a single pot on Scotts® Metro-Mix® 200 soil. Flats are configured with 8 square pots each. Each of the square pots is filled to the top with soil. Each pot (or cell) is sown to produce 9 glufosinate resistant seedlings in a 3×3 array.

The soil is watered to saturation and then plants are grown under standard conditions (i.e., 16 hour light, 8 hour dark cycle; 22° C.; ˜60% relative humidity). No additional water is given.

Digital images of the plants are taken at the onset of visible drought stress symptoms. Images are taken once a day (at the same time of day), until the plants appear dessicated. Typically, four consecutive days of data is captured.

Color analysis is employed for identifying potential drought tolerant lines. Color analysis can be used to measure the increase in the percentage of leaf area that falls into a yellow color bin. Using hue, saturation and intensity data (“HSI”), the yellow color bin consists of hues 35 to 45.

Maintenance of leaf area is also used as another criterion for identifying potential drought tolerant lines, since Arabidopsis leaves wilt during drought stress. Maintenance of leaf area can be measured as reduction of rosette leaf area over time.

Leaf area is measured in terms of the number of green pixels obtained using the LernnaTec imaging system. Activation-tagged and control (e.g., wild-type) plants are grown side by side in flats that contain 72 plants (9 plants/pot). When wilting begins, images are measured for a number of days to monitor the wilting process. From these data wilting profiles are determined based on the green pixel counts obtained over four consecutive days for activation-tagged and accompanying control plants. The profile is selected from a series of measurements over the four day period that gives the largest degree of wilting. The ability to withstand drought is measured by the tendency of activation-tagged plants to resist wilting compared to control plants.

LemnaTec HTSBonitUV software is used to analyze CCD images. Estimates of the leaf area of the Arabidopsis plants are obtained in terms of the number of green pixels. The data for each image is averaged to obtain estimates of mean and standard deviation for the green pixel counts for activation-tagged and wild-type plants. Parameters for a noise function are obtained by straight line regression of the squared deviation versus the mean pixel count using data for all images in a batch. Error estimates for the mean pixel count data are calculated using the fit parameters for the noise function. The mean pixel counts for activation-tagged and wild-type plants are summed to obtain an assessment of the overall leaf area for each image. The four-day interval with maximal wilting is obtained by selecting the interval that corresponds to the maximum difference in plant growth. The individual wilting responses of the activation-tagged and wild-type plants are obtained by normalization of the data using the value of the green pixel count of the first day in the interval. The drought tolerance of the activation-tagged plant compared to the wild-type plant is scored by summing the weighted difference between the wilting response of activation-tagged plants and wild-type plants over day two to day four; the weights are estimated by propagating the error in the data. A positive drought tolerance score corresponds to an activation-tagged plant with slower wilting compared to the wild-type plant. Significance of the difference in wilting response between activation-tagged and wild-type plants is obtained from the weighted sum of the squared deviations.

Lines with a significant delay in yellow color accumulation and/or with significant maintenance of rosette leaf area, when compared to the average of the whole flat, are designated as Phase 1 hits. Phase 1 hits are re-screened in duplicate under the same assay conditions. When either or both of the Phase 2 replicates show a significant difference (score of greater than 0.9) from the whole flat mean, the line is then considered a validated drought tolerant line.

### Example 3

### Identification of Activation-Tagged Genes

Genes flanking the T-DNA insert in drought tolerant lines are identified using one, or both, of the following two standard procedures: (1) thermal asymmetric interlaced (TAIL) PCR (Liu et al., (1995), Plant J. 8:457-63); and (2) SAIFF PCR (Siebert et al., (1995) Nucleic Acids Res. 23:1087-1088). In lines with complex multimerized T-DNA inserts, TAIL PCR and SAIFF PCR may both prove insufficient to identify candidate genes. In these cases, other procedures, including inverse PCR, plasmid rescue and/or genomic library construction, can be employed.

A successful result is one where a single TAIL or SAIFF PCR fragment contains a T-DNA border sequence and Arabidopsis genomic sequence.

Once a tag of genomic sequence flanking a T-DNA insert is obtained, candidate genes are identified by alignment to publicly available Arabidopsis genome sequence.

Specifically, the annotated gene nearest the 35S enhancer elements/T-DNA RB are candidates for genes that are activated.

To verify that an identified gene is truly near a T-DNA and to rule out the possibility that the TAIL/SAIFF fragment is a chimeric cloning artifact, a diagnostic PCR on genomic DNA is done with one oligo in the T-DNA and one oligo specific for the candidate gene. Genomic DNA samples that give a PCR product are interpreted as representing a T-DNA insertion. This analysis also verifies a situation in which more than one insertion event occurs in the same line, e.g., if multiple differing genomic fragments are identified in TAIL and/or SAIFF PCR analyses.

### Example 4A

### Identification of Activation-Tagged MATE-Efflux Polypeptide Gene

An activation-tagged line (No. 102739) showing drought tolerance was further analyzed. DNA from the line was extracted, and genes flanking the T-DNA insert in the mutant line were identified using SAIFF PCR (Siebert et al., Nucleic Acids Res. 23:1087-1088 (1995)). A PCR amplified fragment was identified that contained T-DNA border sequence and Arabidopsis genomic sequence. Genomic sequence flanking the T-DNA insert was obtained, and the candidate gene was identified by alignment to the completed Arabidopsis genome. For a given T-DNA integration event, the annotated gene nearest the 35S enhancer elements/T-DNA RB was the candidate for gene that is activated in the line. In the case of line 102739, the 35S enhancer insert inserted 3′ to At2g04090 with the right border (RB) pointing towards the ORF (open reading frame) encoding a MATE-efflux polypeptide.

### Example 4B

### Assay for Expression Level of Candidate Drought Tolerance Genes

A functional activation-tagged allele should result in either up-regulation of the candidate gene in tissues where it is normally expressed, ectopic expression in tissues that do not normally express that gene, or both.

Expression levels of the candidate genes in the cognate mutant line vs. wild-type are compared. A standard RT-PCR procedure, such as the QuantiTect® Reverse Transcription Kit from Qiagen®, is used. RT-PCR of the actin gene is used as a control to show that the amplification and loading of samples from the mutant line and wild-type are similar.

Assay conditions are optimized for each gene. Expression levels are checked in mature rosette leaves. If the activation-tagged allele results in ectopic expression in other tissues (e.g., roots), it is not detected by this assay. As such, a positive result is useful but a negative result does not eliminate a gene from further analysis.

### Example 5

### Validation of Arabidopsis Candidate Gene At204090 (MATE-Efflux Polypeptide) Via Transformation into Arabidopsis

Candidate genes can be transformed into Arabidopsis and overexpressed under the 35S promoter. If the same or similar phenotype is observed in the transgenic line as in the parent activation-tagged line, then the candidate gene is considered to be a validated “lead gene” in Arabidopsis.

The candidate Arabidopsis MATE-efflux polypeptide gene (At2g04090; SEQ ID NO:17; NCBI GI No. 18395670) was tested for its ability to confer drought tolerance in the following manner.

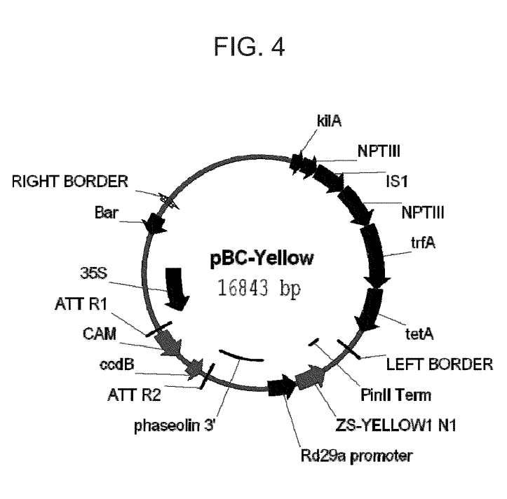

A 16.8-kb T-DNA based binary vector, called pBC-yellow (SEQ ID NO:4; FIG. 4), was constructed with a 1.3-kb 35 S promoter immediately upstream of the INVITROGEN™ GATEWAY® C1 conversion insert. The vector also contains the RD29a promoter driving expression of the gene for ZS-Yellow (INVITROGEN™), which confers yellow fluorescence to transformed seed.

The At2g04090 genomic region was amplified by RT-PCR with the following primers:

(1) At2g04090-5′attB forward primer
(SEQ ID NO. 12):
TTAAACAAGTTTGTACAAAAAAGCAGGCTCAACAATGGAAGATCCAC
TTTTATTG
(2) At2g04090-3′attB reverse primer
(SEQ ID NO: 13):
TTAAACCACTTTGTACAAGAAAGCTGGGTTCAGTATGGGGTAAAAAA
AAG

The forward primer contains the attB1 sequence (ACAAGTTTGTACAAAAAAGCAGGCT; SEQ ID NO:10) and a consensus Kozak sequence (CAACA) adjacent to the first 21 nucleotides of the protein-coding region, beginning with the ATG start codon.

The reverse primer contains the attB2 sequence (ACCACTTTGTACAAGAAAGCTGGGT; SEQ ID NO:11) adjacent to the reverse complement of the last 21 nucleotides of the protein-coding region, beginning with the reverse complement of the stop codon, as identified in SEQ ID NO:17.

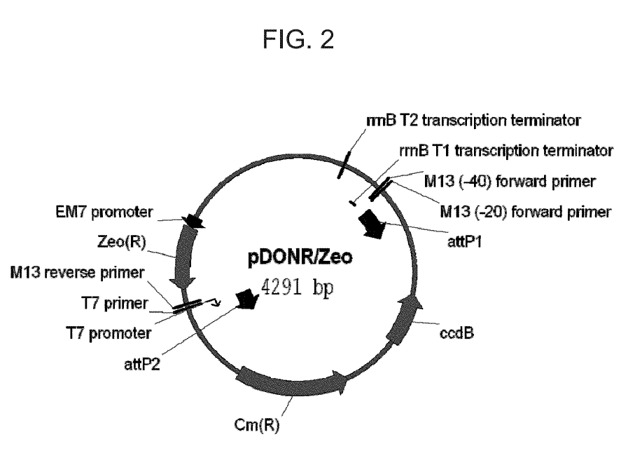

Using the INVITROGEN™ GATEWAY® CLONASE™ technology, a BP Recombination Reaction was performed with pDONR™/Zeo (SEQ ID NO:2; FIG. 2). This process removed the bacteria lethal ccdB gene, as well as the chloramphenicol resistance gene (CAM) from pDONR™/Zeo and directionally cloned the PCR product with flanking attB1 and attB2 sites creating an entry clone, pDONR™/Zeo-At2g04090. This entry clone was used for a subsequent LR Recombination Reaction with a destination vector, as follows.

A 16.8-kb T-DNA based binary vector (destination vector), called pBC-yellow (SEQ ID NO:4; FIG. 4), was constructed with a 1.3-kb 35S promoter immediately upstream of the INVITROGEN™ GATEWAY® C1 conversion insert, which contains the bacterial lethal ccdB gene as well as the chloramphenicol resistance gene (CAM) flanked by attR1 and attR2 sequences. The vector also contains the RD29a promoter driving expression of the gene for ZS-Yellow (INVITROGEN™), which confers yellow fluorescence to transformed seed. Using the INVITROGEN™ GATEWAY® technology, an LR Recombination Reaction was performed on the pDONR™/Zeo-At2g04090entry done, containing the directionally cloned PCR product, and pBC-yellow. This allowed for rapid and directional cloning of the candidate gene behind the 35S promoter in pBC-yellow to create the 35S promoter::At2g04090 expression construct, pBC-Yellow-At2g04090.

Applicants then introduced the 35S promoter::At2g04090 expression construct into wild-type Arabidopsis ecotype Col-0, using the same Agrobacterium-mediated transformation procedure described in Example 1. Transgenic T1 seeds were selected by yellow fluorescence, and T1 seeds were plated next to wild-type seeds and grown under water limiting conditions. Growth conditions and imaging analysis were as described in Example 2. It was found that the original drought tolerance phenotype from activation tagging could be recapitulated in wild-type Arabidopsis plants that were transformed with a construct where At2g04090 was directly expressed by the 35S promoter. The drought tolerance score, as determined by the method of Example 2, was 2.3.

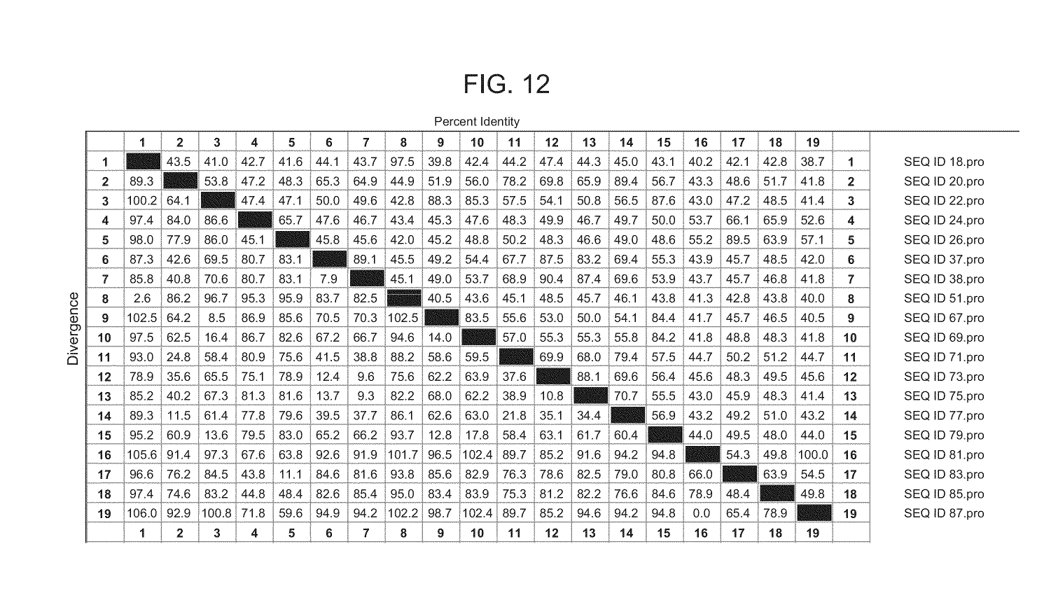

Subsequent to validation of the nucleotide sequence (SEQ ID NO:17) encoding the protein having the amino acid sequence presented in NCBI GI NO. 15228085 (SEQ ID NO:18), a new annotation of the At2g04090 locus was identified which presented NCBI GI NO. 334184134 (SEQ ID NO:51), an updated version of the predicted amino acid sequence for this protein. The corresponding mRNA sequence is presented as NCBI GI NO. 334184133 (SEQ ID NO:50). The corresponding genomic sequence for At2g04090 that encodes both the mRNA sequence of NCBI GI NO. 334184133 (SEQ ID NO:50) and the introns within that sequence is presented in TAIR Accession NO. 6530301899 (SEQ ID NO:103). A multiple alignment of SEQ ID NO:17, SEQ ID NO:50 and SEQ ID NO:103 indicates that the earlier version of the AT-MATE-EP (SEQ ID NO:17) is a consequence of a 3′ intron not being correctly identified. The updated version of the AT-MATE-EP sequence (SEQ ID NO:50) correctly accounts for this 3′ intron. The corresponding amino acids sequences of the two versions of the AT-MATE-EP proteins differ in the carboxy-terminal end, with the amino acid sequence of SEQ ID NO:18 having an artificial final 20 amino acids, instead of having the authentic carboxy-terminal 14 amino acids of SEQ ID NO:51. SEQ ID NO:18 and SEQ ID NO:51 have 97.5% amino acid sequence identity using either the Clustal V (FIG. 12) or the Clustal W method of alignment, with the respective default parameters.

### Example 6

### Preparation of cDNA Libraries and Isolation and Sequencing of cDNA Clones

cDNA libraries may be prepared by any one of many methods available. For example, the cDNAs may be introduced into plasmid vectors by first preparing the cDNA libraries in UNI-ZAP™ XR vectors according to the manufacturer's protocol (Stratagene Cloning Systems, La Jolla, Calif.). The UNI-ZAP™ XR libraries are converted into plasmid libraries according to the protocol provided by Stratagene. Upon conversion, cDNA inserts will be contained in the plasmid vector pBLUESCRIPT®. In addition, the cDNAs may be introduced directly into precut BLUESCRIPT® II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBLUESCRIPT® plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system places unique binding sites randomly throughout a population of large DNA molecules. The transposed DNA is then used to transform DH10B electro-competent cells (GIBCO BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones are randomly selected from each transposition reaction, plasmid DNAs are prepared via alkaline lysis, and templates are sequenced (ABI PRISM® dye-terminator ReadyReaction mix) outward from the transposition event site, utilizing unique primers specific to the binding sites within the transposon.

Sequence data is collected (ABI PRISM® Collections) and assembled using Phred and Phrap (Ewing et al. (1998) Genome Res. 8:175-185; Ewing and Green (1998) Genome Res. 8:186-194). Phred is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (Gordon et al. (1998) Genome Res. 8:195-202).

In some of the clones the cDNA fragment may correspond to a portion of the 3′-terminus of the gene and does not cover the entire open reading frame. In order to obtain the upstream information one of two different protocols is used. The first of these methods results in the production of a fragment of DNA containing a portion of the desired gene sequence while the second method results in the production of a fragment containing the entire open reading frame. Both of these methods use two rounds of PCR amplification to obtain fragments from one or more libraries. The libraries some times are chosen based on previous knowledge that the specific gene should be found in a certain tissue and sometimes are randomly-chosen. Reactions to obtain the same gene may be performed on several libraries in parallel or on a pool of libraries. Library pools are normally prepared using from 3 to 5 different libraries and normalized to a uniform dilution. In the first round of amplification both methods use a vector-specific (forward) primer corresponding to a portion of the vector located at the 5′-terminus of the clone coupled with a gene-specific (reverse) primer. The first method uses a sequence that is complementary to a portion of the already known gene sequence while the second method uses a gene-specific primer complementary to a portion of the 3-untranslated region (also referred to as UTR). In the second round of amplification a nested set of primers is used for both methods. The resulting DNA fragment is ligated into a pBLUESCRIPT® vector using a commercial kit and following the manufacturer's protocol. This kit is selected from many available from several vendors including INVITROGEN™ (Carlsbad, Calif.), Promega Biotech (Madison, Wis.), and GIBCO-BRL (Gaithersburg, Md.). The plasmid DNA is isolated by alkaline lysis method and submitted for sequencing and assembly using Phred/Phrap, as above.

An alternative method for preparation of cDNA Libraries and obtainment of sequences can be the following. mRNAs can be isolated using the Qiagen® RNA isolation kit for total RNA isolation, followed by mRNA isolation via attachment to oligo(dT) Dynabeads from Invitrogen (Life Technologies, Carlsbad, Calif.), and sequencing libraries can be prepared using the standard mRNA-Seq kit and protocol from Illumina, Inc. (San Diego, Calif.). In this method, mRNAs are fragmented using a Zna2 solution, reverse transcribed into cDNA using random primers, end repaired to create blunt end fragments, 3′ A-tailed, and ligated with Illumina paired-end library adaptors. Ligated cDNA fragments can then be PCR amplified using Illumina paired-end library primers, and purified PCR products can be checked for quality and quantity on the Agilent Bioanalyzer DNA 1000 chip prior to sequencing on the Genome Analyzer II equipped with a paired end module.

Reads from the sequencing runs can be soft-trimmed prior to assembly such that the first base pair of each read with an observed FASTQ quality score lower than 15 and all subsequent bases are clipped using a Python script. The Velvet assembler (Zerbino et al, Genome Research 18:821-9 (2008)) can be run under varying kmer and coverage cutoff parameters to produce several putative assemblies along a range of stringency. The contiguous sequences (contigs) within those assemblies can be combined into clusters using Vmatch software (available on the Vmatch website) such that contigs which are identified as substrings of longer contigs are grouped and eliminated, leaving a non-redundant set of longest “sentinel” contigs. These non-redundant sets can be used in alignments to homologous sequences from known model plant species.

### Example 7

### Identification of cDNA Clones

cDNA clones encoding the polypeptide of interest can be identified by conducting BLAST (Basic Local Alignment Search Tool; Altschul et al. (1993) J. Mol. Biol. 215:403-410; see also the explanation of the BLAST algorithm on the world wide web site for the National Center for Biotechnology Information at the National Library of Medicine of the National Institutes of Health) searches for similarity to amino acid sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The DNA sequences from clones can be translated in all reading frames and compared for similarity to all publicly available protein sequences contained in the “nr” database using the BLASTX algorithm (Gish and States (1993) Nat. Genet. 3:266-272) provided by the NCBI. The polypeptides encoded by the cDNA sequences can be analyzed for similarity to all publicly available amino acid sequences contained in the “nr” database using the BLASTP algorithm provided by the National Center for Biotechnology Information (NCBI). For convenience, the P-value (probability) or the E-value (expectation) of observing a match of a cDNA-encoded sequence to a sequence contained in the searched databases merely by chance as calculated by BLAST are reported herein as “pLog” values, which represent the negative of the logarithm of the reported P-value or E-value. Accordingly, the greater the pLog value, the greater the likelihood that the cDNA-encoded sequence and the BLAST “hit” represent homologous proteins.

ESTs sequences can be compared to the Genbank database as described above. ESTs that contain sequences more 5- or 3-prime can be found by using the BLASTN algorithm (Altschul et al (1997) Nucleic Acids Res. 25:3389-3402.) against the DUPONT™ proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described above. Homologous genes belonging to different species can be found by comparing the amino acid sequence of a known gene (from either a proprietary source or a public database) against an EST database using the TBLASTN algorithm. The TBLASTN algorithm searches an amino acid query against a nucleotide database that is translated in all 6 reading frames. This search allows for differences in nucleotide codon usage between different species, and for codon degeneracy.

In cases where the sequence assemblies are in fragments, the percent identity to other homologous genes can be used to infer which fragments represent a single gene. The fragments that appear to belong together can be computationally assembled such that a translation of the resulting nucleotide sequence will return the amino acid sequence of the homologous protein in a single open-reading frame. These computer-generated assemblies can then be aligned with other polypeptides of the invention.

### Example 8

### Characterization of cDNA Clones Encoding MATE-Efflux Polypeptides

cDNA libraries representing mRNAs from various tissues of Sugar Beet, Canola, Maize, Rice, Soybean, Wheat and Catmint were prepared and cDNA clones encoding MATE-efflux polypeptides were identified. MATE-efflux polypeptides were also identified from two exotic plant species, Paspalum notatum, commonly called Bahia grass, and Eragrostis nindensis, also called resurrection grass. These are included in Table 1. Mining of homologs from resurrection and Bahia grass was done by performing a TBLASTN of the Arabidopsis MATE-EP genes, and the identified maize MATE-EP homologs against the Bahia and resurrection grass assemblies. The resulting hits were translated based on the blast alignments and the translations were aligned with the other known MATE-EP polypeptides.

The characteristics of the libraries are described below.

TABLE 2
cDNA Libraries from Maize

| Library* | Description | Clone |
|---|---|---|
| cfp6n | Maize Leaf and Seed pooled, | cfp6n.pk010.h3, |
| | Full-length enriched normalized | cfp6n.pk009.n19 |
| cfp1n | Maize Tassel V7 to V12 pooled, | cfp1n.pk004.c4 |
| | Full-length enriched normalized | |
| cfp5n | Maize Kernel, pooled stages, | cfp5n.pk002.e2 |
| | Full-length enriched, normalized | |

*Libraries normalized essentially as described in U.S. Pat. No. 5,482,845

The BLAST search using the sequences from clones listed in Table 2 revealed similarity of the polypeptides encoded by the cDNAs to the MATE-efflux polypeptides from various organisms. As shown in Table 3 and FIGS. 11A-11F, certain cDNAs encoded polypeptides similar to MATE-efflux polypeptide from Arabidopsis (GI No. 15228085, SEQ ID NO:18; and NCBI GI NO. 334184134, SEQ ID NO:51)

Shown in Table 3 (non-patent literature) and Table 4 (patent literature) are the BLASTP results for the amino acid sequences derived from the nucleotide sequences of the entire cDNA inserts (“Full-Insert Sequence” or “FIS”) of the clones listed in Table 2. A cDNA insert that encodes an entire or functional protein is termed a “Complete Gene Sequence” (“CGS”). Also shown in Tables 3 and 4 are the percent sequence identity values for each pair of amino add sequences using the Clustal V method of alignment with default parameters.

TABLE 3
BLASTP Results for MATE-Efflux Polypeptides

| | | BLASTP | Percent |
|---|---|---|---|
| Sequence | NCBI GI No. | pLog of | Sequence |
| (SEQ ID NO) | (SEQ ID NO) | E-value | Identity |
| cfp6n.pk010.h3 (FIS) | 195650919 | >180 | 100 |
| (SEQ ID NO: 20) | (SEQ ID NO: 28) | | |
| cfp1n.pk004.c4 (FIS) | 242041995 | >180 | 89.9 |
| (SEQ ID NO: 22) | (SEQ ID NO: 30) | | |
| cfp6n.pk009.n19 (FIS) | 195619754 | >180 | 100.0 |
| (SEQ ID NO: 24) | (SEQ ID NO: 32) | | |
| cfp5n.pk002.e2 (FIS) | 223949561 | >180 | 100 |
| (SEQ ID NO: 26) | (SEQ ID NO: 34) | | |
| AC187156 | 242088755 | >180 | 89.1 |
| (SEQ ID NO: 37) | (SEQ ID NO: 38) | | |
| wlp1c.pk006.j5 | 194701508 | >180 | 89.3 |
| (SEQ ID NO: 67) | (SEQ ID NO: 96) | | |
| En_NODE_45314 | 326518786 | >180 | 86.2 |
| (SEQ ID NO: 69) | (SEQ ID NO: 65) | | |
| En_NODE_19917 | 56784891 | >180 | 80.8 |
| (SEQ ID NO: 71) | (SEQ ID NO: 90) | | |
| En_NODE_1677 | 215707242 | >180 | 72.5 |
| (SEQ ID NO: 73) | (SEQ ID NO: 92) | | |
| Pn_NODE_53729 | 215740571 | >180 | 78.4 |
| (SEQ ID NO: 75) | (SEQ ID NO: 94) | | |
| Pn_NODE_31640 | 195650919 | >180 | 89.4 |
| (SEQ ID NO: 77) | (SEQ ID NO: 28) | | |
| Pn_NODE_155338 | 194701508 | >180 | 87.6 |
| (SEQ ID NO: 79) | (SEQ ID NO: 96) | | |
| Pn_NODE_21180 | 194689564 | >180 | 53.7 |
| (SEQ ID NO: 81) | (SEQ ID NO: 98) | | |
| Pn_NODE_39122 | 223949561 | >180 | 89.5 |
| (SEQ ID NO: 83) | (SEQ ID NO: 34) | | |
| Pn_NODE_200639 | 195613120 | >180 | 88.6 |
| (SEQ ID NO: 85) | (SEQ ID NO: 101) | | |

TABLE 4
BLASTP Results for MATE-Efflux Polypeptides

| | | BLASTP | Percent |
|---|---|---|---|
| Sequence | Reference | pLog of | Sequence |
| (SEQ ID NO) | (SEQ ID NO) | E-value | Identity |
| At2g04090 | SEQ ID NO: 30086 of | >180 | 100 |
| (SEQ ID NO: 18) | U.S. Pat. No. 7,569,389 | | |
| | (SEQ ID NO: 27) | | |
| cfp6n.pk010.h3 (FIS) | SEQ ID NO: 8539 of | >180 | 100 |
| (SEQ ID NO: 20) | U.S. Pat. No. 7,569,389 | | |
| | (SEQ ID NO: 29) | | |
| cfp1n.pk004.c4 (FIS) | SEQ ID NO: 17653 of | >180 | 99.8 |
| (SEQ ID NO: 22) | US20090070897 | | |
| | (SEQ ID NO: 31) | | |
| cfp6n.pk009.n19 (FIS) | SEQ ID NO: 8873 of | >180 | 100 |
| (SEQ ID NO: 24) | U.S. Pat. No. 7,569,389 | | |
| | (SEQ ID NO: 33) | | |
| cfp5n.pk002.e2 (FIS) | SEQ ID NO: 93375 of | >180 | 97.4 |
| (SEQ ID NO: 26) | WO2008034648 | | |
| | (SEQ ID NO: 35) | | |
| AC187156 | SEQ ID NO: 32358 of | >180 | 77.6 |
| (SEQ ID NO: 37) | US20060107345 | | |
| | (SEQ ID NO: 39) | | |
| wlp1c.pk006.j5 | SEQ ID NO: 26320 of | >180 | 63.9 |
| (SEQ ID NO: 67) | US20100083407 | | |
| | (SEQ ID NO: 102) | | |
| En_NODE_45314 | SEQ ID NO: 11204 of | >180 | 86.7 |
| (SEQ ID NO: 69) | US20110167514 | | |
| | (SEQ ID NO: 88) | | |
| En_NODE_19917 | SEQ ID NO: 54943 of | >180 | 80.8 |
| (SEQ ID NO: 71) | US20060123505 | | |
| | (SEQ ID NO: 89) | | |
| En_NODE_1677 | SEQ ID NO: 52182 of | >180 | 72.5 |
| (SEQ ID NO: 73) | US20060123503 | | |
| | (SEQ ID NO: 91) | | |
| Pn_NODE_53729 | SEQ ID NO: 29593 of | >180 | 88.9 |
| (SEQ ID NO: 75) | US20110167514 | | |
| | (SEQ ID NO: 93) | | |
| Pn_NODE_31640 | SEQ ID NO: 238224 of | >180 | 89.4 |
| (SEQ ID NO: 77) | US20110214206 | | |
| | (SEQ ID NO: 95) | | |
| Pn_NODE_155338 | SEQ ID NO: 11204 of | >180 | 89 |
| (SEQ ID NO: 79) | US20110167514 | | |
| | (SEQ ID NO: 88) | | |
| Pn_NODE_21180 | SEQ ID NO: 155433 of | >180 | 86.7 |
| (SEQ ID NO: 81) | US20110131679 | | |
| | (SEQ ID NO: 97) | | |
| Pn_NODE_39122 | SEQ ID NO: 8544 of | >180 | 90.5 |
| (SEQ ID NO: 83) | US20100083407 | | |
| | (SEQ ID NO: 99) | | |
| Pn_NODE_200639 | SEQ ID NO: 205649 of | >180 | 88.6 |
| (SEQ ID NO: 85) | US20110214206 | | |
| | (SEQ ID NO: 100) | | |

FIGS. 11A-11F present an alignment of the amino acid sequences of MATE-efflux polypeptides set forth in SEQ ID NOs: 18, 20, 22, 24, 26, 37, 38, 51, 67, 69, 71, 73, 75, 77, 79, 81, 83, 85 and 87. FIG. 12 presents the percent sequence identities and divergence values for each sequence pair presented in FIGS. 11A-11F.

Sequence alignments and percent identity calculations were performed using the Megalign® program of the LASERGENE® bioinformatics computing suite (DNASTAR®. Inc., Madison, Wis.). Multiple alignment of the sequences was performed using the Clustal V method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE=1, GAP PENALTY=3, WINDOW=5 and DIAGONALS SAVED=5.

Sequence alignments and BLAST scores and probabilities indicate that the nucleic acid fragments comprising the instant cDNA clones encode MATE-efflux polypeptides.

Other MATE-efflux polypeptide sequences are given in Table 5, below. These sequences are encompassed in the present invention.

TABLE 5
MATE-EP Homologs

| No. | Species | NCBI GI No. |
|---|---|---|
| 1 | Vitis vinifera | 225424132 |
| 2 | Populus trichocarpa | 224108371 |
| 3 | Ricinus communis | 255582915 |
| 4 | Ricinus communis | 255582919 |
| 5 | Populus trichocarpa | 224108375 |
| 6 | Populus trichocarpa | 224101797 |
| 7 | Ricinus communis | 255582921 |
| 8 | Vitis vinifera | 225424130 |
| 9 | Ricinus communis | 255582923 |
| 10 | Populus trichocarpa | 224077218 |
| 11 | Ricinus communis | 255574294 |
| 12 | Nicotiana tabacum | 219921318 |
| 13 | Vitis vinifera | 147782271 |
| 14 | Populus trichocarpa | 224079377 |
| 15 | Populus trichocarpa | 224065226 |
| 16 | Populus trichocarpa | 224065228 |
| 17 | Ricinus communis | 255574300 |
| 18 | Vitis vinifera | 225456065 |
| 19 | Sorghum bicolor | 242096986 |
| 20 | Sorghum bicolor | 242095754 |
| 21 | Sorghum bicolor | 242072630 |
| 22 | Sorghum bicolor | 242064864 |
| 23 | Sorghum bicolor | 242064866 |
| 24 | Sorghum bicolor | 242087587 |
| 25 | Sorghum bicolor | 242080875 |
| 26 | Sorghum bicolor | 242090209 |
| 27 | Sorghum bicolor | 242061364 |
| 28 | Oryza sativa | 297606478 |
| 29 | Oryza sativa | 115468176 |
| 30 | Oryza sativa | 115468182 |
| 31 | Oryza sativa | 215769464 |
| 32 | Oryza sativa | 110288754 |
| 33 | Oryza sativa | 15217298 |
| 34 | Oryza sativa | 115481600 |
| 35 | Arabidopsis thaliana | 30697399 |
| 36 | Arabidopsis thaliana | 42562999 |
| 37 | Arabidopsis thaliana | 15217763 |
| 38 | Arabidopsis thaliana | 15237158 |
| 39 | Arabidopsis thaliana | 240254581 |
| 40 | Glycine max | 356573950 |
| 41 | Glycine max | 356513977 |
| 42 | Glycine max | 356531168 |
| 43 | Glycine max | 356527876 |
| 44 | Glycine max | 356529541 |
| 45 | Glycine max | 356520633 |
| 46 | Glycine max | 356529535 |

### Example 9

### Preparation of a Plant Expression Vector Containing a Homolog to the Arabidopsis Lead Gene

Sequences homologous to the Arabidopsis AT-MATE-efflux polypeptide can be identified using sequence comparison algorithms such as BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health). Sequences encoding homologous MATE-efflux polypeptides can be PCR-amplified by either of the following methods.

Method 1 (RNA-based): If the 5′ and 3′ sequence information for the protein-coding region, or the 5′ or 3′ UTR, of a gene encoding a MATE-efflux polypeptide homolog is available, gene-specific primers can be designed as outlined in Example 5. RT-PCR can be used with plant RNA to obtain a nucleic acid fragment containing the protein-coding region flanked by attB1 (SEQ ID NO:10) and attB2 (SEQ ID NO:11) sequences. The primer may contain a consensus Kozak sequence (CAACA) upstream of the start codon.

Method 2 (DNA-based): Alternatively, if a cDNA clone is available for a gene encoding a MATE-efflux polypeptide homolog, the entire cDNA insert (containing 5′ and 3′ non-coding regions) can be PCR amplified. Forward and reverse primers can be designed that contain either the attB1 sequence and vector-specific sequence that precedes the cDNA insert or the attB2 sequence and vector-specific sequence that follows the cDNA insert, respectively. For a cDNA insert cloned into the vector pBulescript SK+, the forward primer VC062 (SEQ ID NO:14) and the reverse primer VC063 (SEC) ID NO:15) can be used.

Method 3 (genomic DNA): Genomic sequences can be obtained using long range genomic PCR capture. Primers can be designed based on the sequence of the genomic locus and the resulting PCR product can be sequenced. The sequence can be analyzed using the FGENESH (Salamov, A. and Solovyev, V. (2000) Genome Res., 10: 516-522) program, and optionally, can be aligned with homologous sequences from other species to assist in identification of putative introns.

The above methods can be modified according to procedures known by one skilled in the art. For example, the primers of Method 1 may contain restriction sites instead of attB1 and attB2 sites, for subsequent cloning of the PCR product into a vector containing attB1 and attB2 sites. Additionally, Method 2 can involve amplification from a cDNA clone, a lambda clone, a BAC clone or genomic DNA.

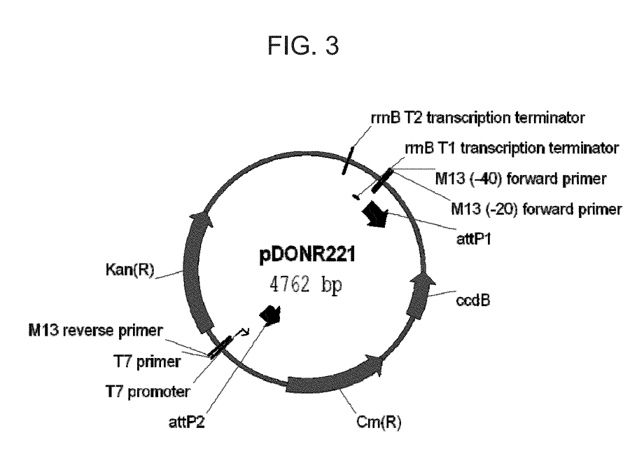

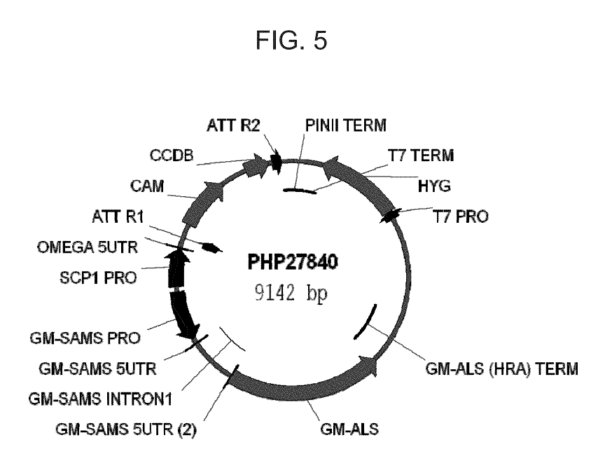

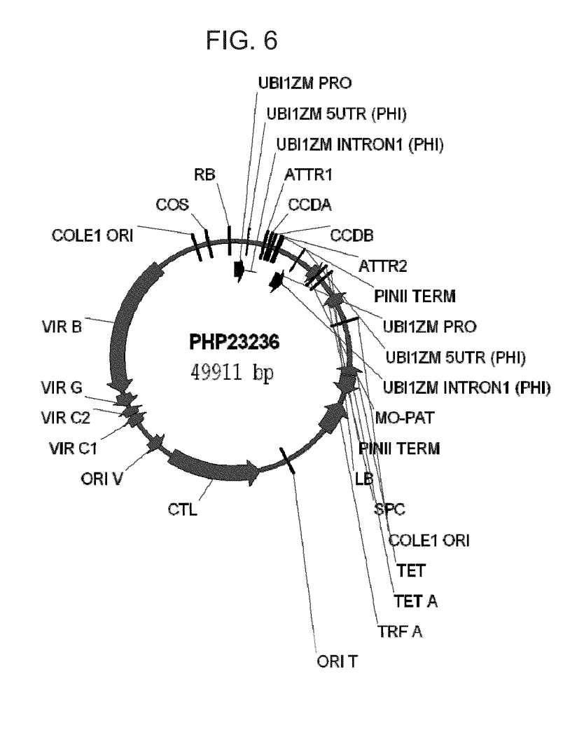

A PCR product obtained by any of the above methods above can be combined with the GATEWAY® donor vector, such as pDONR™/Zeo (INVITROGEN™; FIG. 2; SEQ ID NO:2) or pDONR™221 (INVITROGEN™; FIG. 3; SEQ ID NO:3), using a BP Recombination Reaction. This process removes the bacteria lethal ccdB gene, as well as the chloramphenicol resistance gene (CAM) from pDONR™221 and directionally clones the PCR product with flanking attB1 and attB2 sites to create an entry clone. Using the INVITROGEN™ GATEWAY®CLONASE™ technology, the sequence encoding the homologous MATE-efflux polypeptide from the entry clone can then be transferred to a suitable destination vector, such as pBC-Yellow (FIG. 4; SEQ ID NO:4), PHP27840 (FIG. 5; SEQ ID NO:5) or PHP23236 (FIG. 6; SEQ ID NO:6), to obtain a plant expression vector for use with Arabidopsis, soybean and corn, respectively.

The attP1 and attP2 sites of donor vectors pDONRT™/Zeo or pDONR™221 are shown in FIGS. 2 and 3, respectively. The attR1 and attR2 sites of destination vectors pBC-Yellow, PHP27840 and PHP23236 are shown in FIGS. 4, 5 and 6, respectively.

Alternatively a MultiSite GATEWAY® LR recombination reaction between multiple entry clones and a suitable destination vector can be performed to create an expression vector.

### Example 10

### Preparation of Soybean Expression Vectors and Transformation of Soybean with Validated Arabidopsis Lead Genes

Soybean plants can be transformed to overexpress a validated Arabidopsis lead gene or the corresponding homologs from various species in order to examine the resulting phenotype.

The same GATEWAY® entry clone described in Example 5 can be used to directionally clone each gene into the PHP27840 vector (SEQ ID NO:5; FIG. 5) such that expression of the gene is under control of the SCP1 promoter (International Publication No. 03/033651).

Soybean embryos may then be transformed with the expression vector comprising sequences encoding the instant polypeptides. Techniques for soybean transformation and regeneration have been described in International Patent Publication WO 2009/006276, the contents of which are herein incorporated by reference.

T1 plants can be subjected to a soil-based drought stress. Using image analysis, plant area, volume, growth rate and color analysis can be taken at multiple times before and during drought stress. Overexpression constructs that result in a significant delay in wilting or leaf area reduction, yellow color accumulation and/or increased growth rate during drought stress will be considered evidence that the Arabidopsis gene functions in soybean to enhance drought tolerance.

Soybean plants transformed with validated genes can then be assayed under more vigorous field-based studies to study yield enhancement and/or stability under well-watered and water-limiting conditions.

### Example 11

### Transformation of Maize with Validated Arabidopsis Lead Genes Using Particle Bombardment

Maize plants can be transformed to overexpress a validated Arabidopsis lead gene or the corresponding homologs from various species in order to examine the resulting phenotype.

The same GATEWAY® entry clone described in Example 5 can be used to directionally clone each gene into a maize transformation vector. Expression of the gene in the maize transformation vector can be under control of a constitutive promoter such as the maize ubiquitin promoter (Christensen et al., (1989) Plant Mol. Biol. 12:619-632 and Christensen et al., (1992) Plant Mol. Biol. 18:675-689)

The recombinant DNA construct described above can then be introduced into corn cells by particle bombardment. Techniques for corn transformation by particle bombardment have been described in International Patent Publication WO 2009/006276, the contents of which are herein incorporated by reference.

T1 plants can be subjected to a soil-based drought stress. Using image analysis, plant area, volume, growth rate and color analysis can be taken at multiple times before and during drought stress. Overexpression constructs that result in a significant delay in wilting or leaf area reduction, yellow color accumulation and/or increased growth rate during drought stress will be considered evidence that the Arabidopsis gene functions in maize to enhance drought tolerance.

### Example 12

### Electroporation of Agrobacterium tumefaciens LBA4404

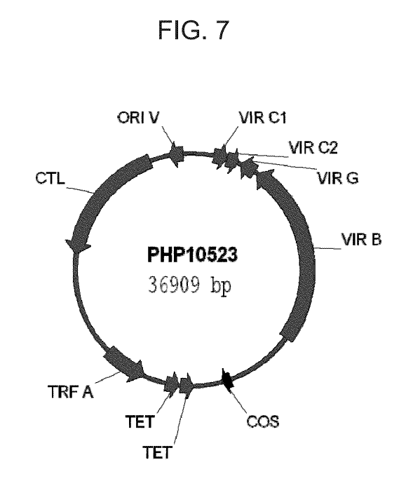

Electroporation competent cells (40 μL), such as Agrobacterium tumefaciens LBA4404 containing PHP10523 (FIG. 7; SEQ ID NO:7), are thawed on ice (20-30 min). PHP10523 contains VIR genes for T-DNA transfer, an Agrobacterium low copy number plasmid origin of replication, a tetracycline resistance gene, and a Cos site for in vivo DNA bimolecular recombination. Meanwhile the electroporation cuvette is chilled on ice. The electroporator settings are adjusted to 2.1 kV. A DNA aliquot (0.5 μL parental DNA at a concentration of 0.2 μg-1.0 μg in low salt buffer or twice distilled H₂O) is mixed with the thawed Agrobacterium tumefaciens LBA4404 cells while still on ice. The mixture is transferred to the bottom of electroporation cuvette and kept at rest on ice for 1-2 min. The cells are electroporated (Eppendorf electroporator 2510) by pushing the “pulse” button twice (ideally achieving a 4.0 millisecond pulse). Subsequently, 0.5 mL of room temperature 2xYT medium (or SOC medium) are added to the cuvette and transferred to a 15 mL snap-cap tube (e.g., FALCON™ tube). The cells are incubated at 28-30° C., 200-250 rpm for 3 h.

Aliquots of 250 μL are spread onto plates containing YM medium and 50 μg/mL spectinomycin and incubated three days at 28-30° C. To increase the number of transformants one of two optional steps can be performed:

Option 1: Overlay plates with 30 μL of 15 mg/mL rifampicin. LBA4404 has a chromosomal resistance gene for rifampicin. This additional selection eliminates it) some contaminating colonies observed when using poorer preparations of LBA4404 competent cells.

Option 2: Perform two replicates of the electroporation to compensate for poorer electrocompetent cells.

Identification of Transformants:

Four independent colonies are picked and streaked on plates containing AB minimal medium and 50 μg/mL spectinomycin for isolation of single colonies. The plates are incubated at 28° C. for two to three days. A single colony for each putative co-integrate is picked and inoculated with 4 mL of 10 g/L bactopeptone, 10 g/L yeast extract, 5 g/L sodium chloride and 50 mg/L spectinomycin. The mixture is incubated for 24 h at 28° C. with shaking. Plasmid DNA from 4 mL of culture is isolated using Qiagen® Miniprep and an optional Buffer PB wash. The DNA is eluted in 30 μL. Aliquots of 2 μL are used to electroporate 20 μL of DH10b+20 μL of twice distilled H₂O as per above. Optionally a 15 μL aliquot can be used to transform 75-100 μL of INVITROGEN™ Library Efficiency DH5α. The cells are spread on plates containing LB medium and 50 μg/mL spectinomycin and incubated at 37° C. overnight.

Three to four independent colonies are picked for each putative co-integrate and inoculated 4 mL of 2xYT medium (10 g/L bactopeptone, 10 g/L yeast extract, 5 g/L sodium chloride) with 50 μg/mL spectinomycin. The cells are incubated at 37° C. overnight with shaking. Next, isolate the plasmid DNA from 4 mL of culture using QIAprep® Miniprep with optional Buffer PB wash (elute in 50 μL). Use 8 μL for digestion with SalI (using parental DNA and PHP10523 as controls). Three more digestions using restriction enzymes BamHI, EcoRI, and HindIII are performed for 4 plasmids that represent 2 putative co-integrates with correct SalI digestion pattern (using parental DNA and PHP10523 as controls). Electronic gels are recommended for comparison.

### Example 13

### Transformation of Maize Using Agrobacterium

Maize plants can be transformed to overexpress a validated Arabidopsis lead gene or the corresponding homologs from various species in order to examine the resulting phenotype.

Agrobacterium-mediated transformation of maize is performed essentially as described by Zhao et al. in Meth. Mol. Biol. 318:315-323 (2006) (see also Zhao et al., Mol. Breed, 8:323-333 (2001) and U.S. Pat. No. 5,981,840 issued Nov. 9, 1999, incorporated herein by reference). The transformation process involves bacterium innoculation, co-cultivation, resting, selection and plant regeneration.

1. Immature Embryo Preparation:

Immature maize embryos are dissected from caryopses and placed in a 2 mL microtube containing 2 mL PHI-A medium.

2. Agrobacterium Infection and Co-Cultivation of Immature Embryos:

2.1 Infection Step:

PHI-A medium of (1) is removed with 1 mL micropipettor, and 1 mL of Agrobacterium suspension is added. The tube is gently inverted to mix. The mixture is incubated for 5 min at room temperature.

2.2 Co-Culture Step:

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is critical for recovering stable transgenic events.

3. Selection of Putative Transgenic Events:

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

4. Regeneration of T0 Plants:

Embryonic tissue propagated on PHI-0 medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

Media for Plant Transformation:
1. PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L Gelrite®, 100 μM acetosyringone (filter-sterilized), pH 5.8.
3 PHI-C: PHI-B without Gelrite® and acetosyringonee, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined. T1 seed can be collected.

Furthermore, a recombinant DNA construct containing a validated Arabidopsis gene can be introduced into an elite maize inbred line either by direct transformation or introgression from a separately transformed line.

Transgenic plants, either inbred or hybrid, can undergo more vigorous field-based experiments to study yield enhancement and/or stability under water limiting and water non-limiting conditions.

Subsequent yield analysis can be done to determine whether plants that contain the validated Arabidopsis lead gene have an improvement in yield performance (under water limiting or non-limiting conditions), when compared to the control (or reference) plants that do not contain the validated Arabidopsis lead gene. Specifically, water limiting conditions can be imposed during the flowering and/or grain fill period for plants that contain the validated Arabidopsis lead gene and the control plants. Plants containing the validated Arabidopsis lead gene would have less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

### Example 14A

### Preparation of Arabidopsis Lead Gene (At2g04090) Expression Vector for Transformation of Maize

Using INVITROGEN™ GATEWAY® technology, an LR Recombination Reaction was performed with an entry clone (pDONR™/Zeo-At2g04090) and a destination vector (PHP28647) to create a precursor plasmid. The precursor plasmid contains the following expression cassettes:

1. Ubiquitin promoter::moPAT::PinII terminator; cassette expressing the PAT herbicide resistance gene used for selection during the transformation process.

2. LTP2 promoter::DS-RED2::PinII terminator; cassette expressing the DS-RED color marker gene used for seed sorting.

3. Ubiquitin promoter::At2g04090::PinII terminator; cassette overexpressing the gene of interest, Arabidopsis AT-MATE-efflux polypeptide.

### Example 14B

### Transformation of Maize with the Arabidopsis Lead Gene (At2g04090) Using Agrobacterium

The AT-MATE-efflux polypeptide expression cassette present in the precursor plasmid can be introduced into a maize inbred line, or a transformable maize line derived from an elite maize inbred line, using Agrobacterium-mediated transformation as described in Examples 12 and 13.

The precursor plasmid can be electroporated into the LBA4404 Agrobacterium strain containing vector PHP10523 (FIG. 7; SEQ ID NO:7) to create a co-integrate vector. The co-integrate vector is formed by recombination of the 2 plasmids, the precursor plasmid and PHP10523, through the COS recombination sites contained on each vector. The co-integrate vector contains the same 3 expression cassettes as above (Example 14A) in addition to other genes (TET, TET, TRFA, ORI terminator, CTL, ORI V, VIR C1, VIR C2, VIR G, VIR B) needed for the Agrobacterium strain and the Agrobacterium-mediated transformation.

### Example 15

### Preparation of the Destination Vector PHP23236 for Transformation into Gaspe Flint Derived Maize Lines

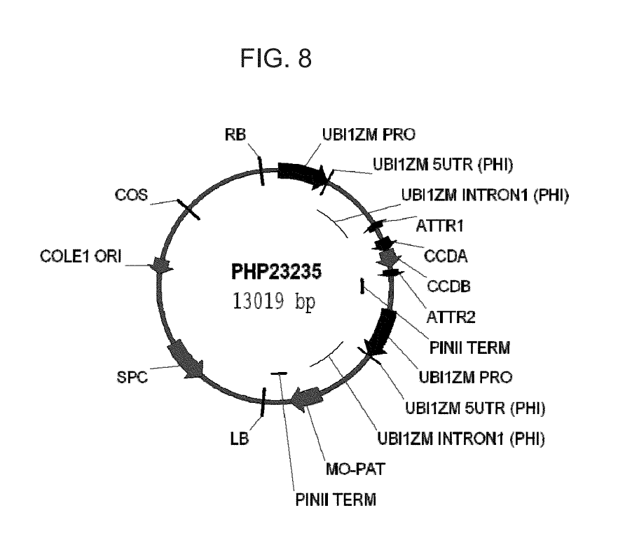

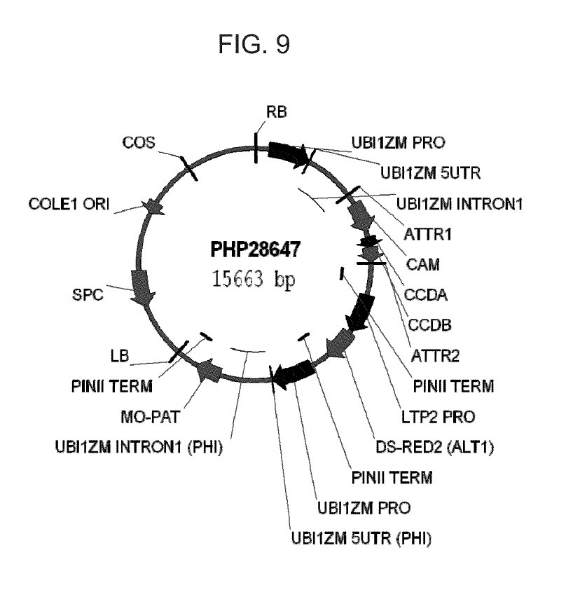

Destination vector PHP23236 (FIG. 6, SEQ ID NO:6) was obtained by transformation of Agrobacterium strain LBA4404 containing plasmid PHP10523 (FIG. 7, SEQ ID NO:7) with plasmid PHP23235 (FIG. 8, SEQ ID NO:8) and isolation of the resulting co-integration product. Destination vector PHP23236, can be used in a recombination reaction with an entry clone as described in Example 16 to create a maize expression vector for transformation of Gaspe Flint-derived maize lines.

### Example 16

### Preparation of Plasmids for Transformation into Gaspe Flint Derived Maize Lines

Using the INVITROGEN™ GATEWAY® LR Recombination technology, the protein-coding region of the candidate gene described in Example 5, pDONR™/Zeo-At2g04090 can be directionally cloned into the destination vector PHP23236 (SEQ ID NO:6; FIG. 6) to create an expression vector. This expression vector contains the protein-coding region of interest, encoding the AT-MATE-efflux polypeptide, under control of the UBI promoter and is a T-DNA binary vector for Agrobacterium-mediated transformation into corn as described, but not limited to, the examples described herein.

Using the INVITROGEN™ GATEWAY® LR Recombination technology, the protein-coding region of the candidate gene described in Example 5, pDONR™/Zeo-At2g04090 can also be directionally cloned into the destination vector PHP29634 to create an expression vector. Destination vector PHP29634 is similar to destination vector PHP23236, however, destination vector PHP29634 has site-specific recombination sites FRT1 and FRT87 and also encodes the GAT4602 selectable marker protein for selection of transformants using glyphosate. This expression vector contains the protein-coding region of interest, encoding the Arabidopsis MATE-efflux polypeptide, under control of the UBI promoter and is a T-DNA binary vector for Agrobacterium-mediated transformation into corn as described, but not limited to, the examples described herein.

### Example 17

### Transformation of Gaspe Flint Derived Maize Lines with a Validated Arabidopsis Lead Gene

Maize plants can be transformed to overexpress the Arabidopsis lead gene or the corresponding homologs from other species in order to examine the resulting phenotype.

Recipient Plants:

Recipient plant cells can be from a uniform maize line having a short life cycle (“fast cycling”), a reduced size, and high transformation potential. Typical of these plant cells for maize are plant cells from any of the publicly available Gaspe Flint (GBF) line varieties. One possible candidate plant line variety is the F1 hybrid of GBF×QTM (Quick Turnaround Maize, a publicly available form of Gaspe Flint selected for growth under greenhouse conditions) disclosed in Tomes et al. U.S. Patent Application Publication No. 2003/0221212. Transgenic plants obtained from this line are of such a reduced size that they can be grown in four inch pots (¼ the space needed for a normal sized maize plant) and mature in less than 2.5 months. (Traditionally 3.5 months is required to obtain transgenic T0 seed once the transgenic plants are acclimated to the greenhouse.) Another suitable line is a double haploid line of GS3 (a highly transformable line) X Gaspe Flint. Yet another suitable line is a transformable elite inbred line carrying a transgene which causes early flowering, reduced stature, or both.

Transformation Protocol:

Any suitable method may be used to introduce the transgenes into the maize cells, including but not limited to inoculation type procedures using Agrobacterium based vectors. Transformation may be performed on immature embryos of the recipient (target) plant.

Precision Growth and Plant Tracking:

The event population of transgenic (T0) plants resulting from the transformed maize embryos is grown in a controlled greenhouse environment using a modified randomized block design to reduce or eliminate environmental error. A randomized block design is a plant layout in which the experimental plants are divided into groups (e.g., thirty plants per group), referred to as blocks, and each plant is randomly assigned a location with the block.

For a group of thirty plants, twenty-four transformed, experimental plants and six control plants (plants with a set phenotype) (collectively, a “replicate group”) are placed in pots which are arranged in an array (a.k.a. a replicate group or block) on a table located inside a greenhouse. Each plant, control or experimental, is randomly assigned to a location with the block which is mapped to a unique, physical greenhouse location as well as to the replicate group. Multiple replicate groups of thirty plants each may be grown in the same greenhouse in a single experiment. The layout (arrangement) of the replicate groups should be determined to minimize space requirements as well as environmental effects within the greenhouse. Such a layout may be referred to as a compressed greenhouse layout.

An alternative to the addition of a specific control group is to identify those transgenic plants that do not express the gene of interest. A variety of techniques such as RT-PCR can be applied to quantitatively assess the expression level of the introduced gene. T0 plants that do not express the transgene can be compared to those which do.

Each plant in the event population is identified and tracked throughout the evaluation process, and the data gathered from that plant is automatically associated with that plant so that the gathered data can be associated with the transgene carried by the plant. For example, each plant container can have a machine readable label (such as a Universal Product Code (UPC) her code) which includes information about the plant identity, which in turn is correlated to a greenhouse location so that data obtained from the plant can be automatically associated with that plant.

Alternatively any efficient, machine readable, plant identification system can be used, such as two-dimensional matrix codes or even radio frequency identification tags (RFID) in which the data is received and interpreted by a radio frequency receiver/processor. See U.S. Published Patent Application No. 200410122592, incorporated herein by reference.

Phenotypic Analysis Using Three-Dimensional Imaging:

Each greenhouse plant in the T0 event population, including any control plants, is analyzed for agronomic characteristics of interest, and the agronomic data for each plant is recorded or stored in a manner so that it is associated with the identifying data (see above) for that plant. Confirmation of a phenotype (gene effect) can be accomplished in the T1 generation with a similar experimental design to that described above.

The T0 plants are analyzed at the phenotypic level using quantitative, non-destructive imaging technology throughout the plant's entire greenhouse life cycle to assess the traits of interest. A digital imaging analyzer may be used for automatic multi-dimensional analyzing of total plants. The imaging may be done inside the greenhouse. Two camera systems, located at the top and side, and an apparatus to rotate the plant, are used to view and image plants from all sides. Images are acquired from the top, front and side of each plant. All three images together provide sufficient information to evaluate the biomass, size and morphology of each plant.

Due to the change in size of the plants from the time the first leaf appears from the soil to the time the plants are at the end of their development, the early stages of plant development are best documented with a higher magnification from the top. This may be accomplished by using a motorized zoom lens system that is fully controlled by the imaging software.

In a single imaging analysis operation, the following events occur: (1) the plant is conveyed inside the analyzer area, rotated 360 degrees so its machine readable label can be read, and left at rest until its leaves stop moving; (2) the side image is taken and entered into a database; (3) the plant is rotated 90 degrees and again left at rest until its leaves stop moving, and (4) the plant is transported out of the analyzer.

Plants are allowed at least six hours of darkness per twenty four hour period in order to have a normal day/night cycle.

Imaging Instrumentation:

Any suitable imaging instrumentation may be used, including but not limited to light spectrum digital imaging instrumentation commercially available from LemnaTec GmbH of Wurselen, Germany. The images are taken and analyzed with a LemnaTec Scanalyzer HIS LT-0001-2 having a ½″ IT Progressive Scan IEE CCD imaging device. The imaging cameras may be equipped with a motor zoom, motor aperture and motor focus. All camera settings may be made using LemnaTec software. For example, the instrumental variance of the imaging analyzer is less than about 5% for major components and less than about 10% for minor components.

Software:

The imaging analysis system comprises a LemnaTec HTS Bonit software program for color and architecture analysis and a server database for storing data from about 500,000 analyses, including the analysis dates. The original images and the analyzed images are stored together to allow the user to do as much reanalyzing as desired. The database can be connected to the imaging hardware for automatic data collection and storage. A variety of commercially available software systems (e.g. Matlab, others) can be used for quantitative interpretation of the imaging data, and any of these software systems can be applied to the image data set.

Conveyor System:

A conveyor system with a plant rotating device may be used to transport the plants to the imaging area and rotate them during imaging. For example, up to four plants, each with a maximum height of 1.5 m, are loaded onto cars that travel over the circulating conveyor system and through the imaging measurement area. In this case the total footprint of the unit (imaging analyzer and conveyor loop) is about 5 m×5 m.

The conveyor system can be enlarged to accommodate more plants at a time. The plants are transported along the conveyor loop to the imaging area and are analyzed for up to 50 seconds per plant. Three views of the plant are taken. The conveyor system, as well as the imaging equipment, should be capable of being used in greenhouse environmental conditions.

Illumination:

Any suitable mode of illumination may be used for the image acquisition. For example, a top light above a black background can be used. Alternatively, a combination of top- and backlight using a white background can be used. The illuminated area should be housed to ensure constant illumination conditions. The housing should be longer than the measurement area so that constant light conditions prevail without requiring the opening and closing or doors. Alternatively, the illumination can be varied to cause excitation of either transgene (e.g., green fluorescent protein (GFP), red fluorescent protein (RFP)) or endogenous (e.g. Chlorophyll) fluorophores.

Biomass Estimation Based on Three-Dimensional Imaging:

For best estimation of biomass the plant images should be taken from at least three axes, for example, the top and two side (sides 1 and 2) views. These images are then analyzed to separate the plant from the background, pot and pollen control bag (if applicable). The volume of the plant can be estimated by the calculation:

Volume(voxels)=\sqrt{TopArea(pixels)}×\sqrt{Side1Area(pixels)}×\sqrt{Side2Area(pixels)}

In the equation above the units of volume and area are “arbitrary units”. Arbitrary units are entirely sufficient to detect gene effects on plant size and growth in this system because what is desired is to detect differences (both positive-larger and negative-smaller) from the experimental mean, or control mean. The arbitrary units of size (e.g. area) may be trivially converted to physical measurements by the addition of a physical reference to the imaging process. For instance, a physical reference of known area can be included in both top and side imaging processes. Based on the area of these physical references a conversion factor can be determined to allow conversion from pixels to a unit of area such as square centimeters (cm²). The physical reference may or may not be an independent sample. For instance, the pot, with a known diameter and height, could serve as an adequate physical reference.

Color Classification:

The imaging technology may also be used to determine plant color and to assign plant colors to various color classes. The assignment of image colors to color classes is an inherent feature of the LemnaTec software. With other image analysis software systems color classification may be determined by a variety of computational approaches.

For the determination of plant size and growth parameters, a useful classification scheme is to define a simple color scheme including two or three shades of green and, in addition, a color class for chlorosis, necrosis and bleaching, should these conditions occur. A background color class which includes non plant colors in the image (for example pot and soil colors) is also used and these pixels are specifically excluded from the determination of size. The plants are analyzed under controlled constant illumination so that any change within one plant over time, or between plants or different batches of plants (e.g. seasonal differences) can be quantified.

In addition to its usefulness in determining plant size growth, color classification can be used to assess other yield component traits. For these other yield component traits additional color classification schemes may be used. For instance, the trait known as “staygreen”, which has been associated with improvements in yield, may be assessed by a color classification that separates shades of green from shades of yellow and brown (which are indicative of senescing tissues). By applying this color classification to images taken toward the end of the T0 or T1 plants' life cycle, plants that have increased amounts of green colors relative to yellow and brown colors (expressed, for instance, as Green/Yellow Ratio) may be identified. Plants with a significant difference in this Green/Yellow ratio can be identified as carrying transgenes which impact this important agronomic trait.

The skilled plant biologist will recognize that other plant colors arise which can indicate plant health or stress response (for instance anthocyanins), and that other color classification schemes can provide further measures of gene action in traits related to these responses.

Plant Architecture Analysis:

Transgenes which modify plant architecture parameters may also be identified using the present invention, including such parameters as maximum height and width, internodal distances, angle between leaves and stem, number of leaves starting at nodes and leaf length. The LemnaTec system software may be used to determine plant architecture as follows. The plant is reduced to its main geometric architecture in a first imaging step and then, based on this image, parameterized identification of the different architecture parameters can be performed. Transgenes that modify any of these architecture parameters either singly or in combination can be identified by applying the statistical approaches previously described.

Pollen Shed Date:

Pollen shed date is an important parameter to be analyzed in a transformed plant, and may be determined by the first appearance on the plant of an active male flower. To find the male flower object, the upper end of the stem is classified by color to detect yellow or violet anthers. This color classification analysis is then used to define an active flower, which in turn can be used to calculate pollen shed date.

Alternatively, pollen shed date and other easily visually detected plant attributes (e.g. pollination date, first silk date) can be recorded by the personnel responsible for performing plant care. To maximize data integrity and process efficiency this data is tracked by utilizing the same barcodes utilized by the LemnaTec light spectrum digital analyzing device. A computer with a barcode reader, a palm device, or a notebook PC may be used for ease of data capture recording time of observation, plant identifier, and the operator who captured the data.

Orientation of the Plants:

Mature maize plants grown at densities approximating commercial planting often have a planar architecture. That is, the plant has a clearly discernable broad side, and a narrow side. The image of the plant from the broadside is determined. To each plant a well defined basic orientation is assigned to obtain the maximum difference between the broadside and edgewise images. The top image is used to determine the main axis of the plant, and an additional rotating device is used to turn the plant to the appropriate orientation prior to starting the main image acquisition.

### Example 18A

### Evaluation of Gaspe Flint Derived Maize Lines for Drought Tolerance

Transgenic Gaspe Flint derived maize lines containing the candidate gene can be screened for tolerance to drought stress in the following manner.

Transgenic maize plants are subjected to well-watered conditions (control) and to drought-stressed conditions. Transgenic maize plants are screened at the T1 stage or later.

For plant growth, the soil mixture consists of ⅓ TURFACE®, ⅓ SB300 and ⅓ sand. All pots are filled with the same amount of soil ±10 grams. Pots are brought up to 100% field capacity (“FC”) by hand watering. All plants are maintained at 60% FC using a 20-10-20 (N—P—K) 125 ppm N nutrient solution. Throughout the experiment pH is monitored at least three times weekly for each table. Starting at 13 days after planting (DAP), the experiment can be divided into two treatment groups, well watered and reduce watered. All plants comprising the reduced watered treatment are maintained at 40% FC while plants in the well watered treatment are maintained at 80% FC. Reduced watered plants are grown for 10 days under chronic drought stress conditions (40% FC). All plants are imaged daily throughout chronic stress period. Plants are sampled for metabolic profiling analyses at the end of chronic drought period, 22 DAP. At the conclusion of the chronic stress period all plants are imaged and measured for chlorophyll fluorescence. Reduced watered plants are subjected to a severe drought stress period followed by a recovery period, 23-31 DAP and 32-34 DAP respectively. During the severe drought stress, water and nutrients are withheld until the plants reached 8% FC. At the conclusion of severe stress and recovery periods all plants are again imaged and measured for chlorophyll fluorescence. The probability of a greater Student's t Test is calculated for each transgenic mean compared to the appropriate null mean (either segregant null or construct null). A minimum (P<t) of 0.1 is used as a cut off for a statistically significant result.

### Example 18B

### Evaluation of Maize Lines for Drought Tolerance

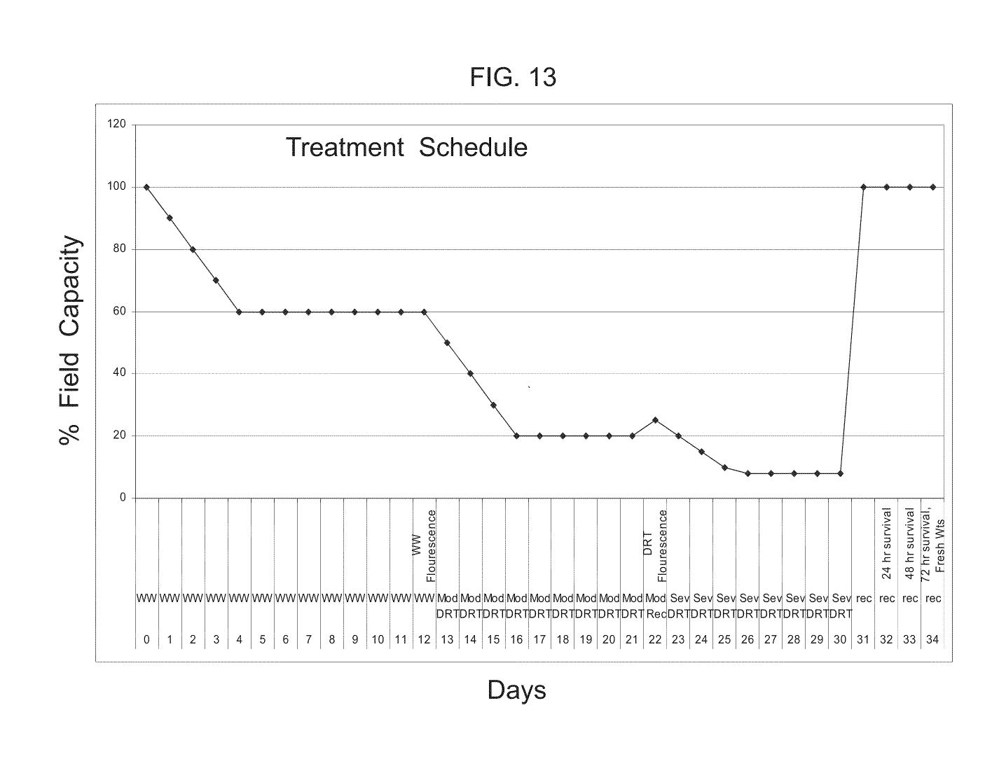

Lines with Enhanced Drought Tolerance can also be screened using the following method (see also FIG. 13 for treatment schedule):

Transgenic maize seedlings are screened for drought tolerance by measuring chlorophyll fluorescence performance, biomass accumulation, and drought survival. Transgenic plants are compared against the null plant (i.e., not containing the transgene). Experimental design is a Randomized Complete Block and Replication consist of 13 positive plants from each event and a construct null (2 negatives each event).

Plant are grown at well watered (WW) conditions 60% Field Capacity (% FC) to a three leaf stage. At the three leaf stage and under WW conditions the first fluorescence measurement is taken on the uppermost fully extended leaf at the inflection point, in the leaf margin and avoiding the mid rib. This is followed by imposing a moderate drought stress (FIG. 13, day 13, MOD DRT) by maintaining 20% FC for duration of 9 to 10 days. During this stress treatment leaves may appear gray and rolling may occur. At the end of MOD DRT period, plants are recovered (MOD rec) by increasing to 25% FC. During this time, leaves will begin to unroll. This is a time sensitive step that may take up to 1 hour to occur and can be dependent upon the construct and events being tested. When plants appear to have recovered completed (leaves unrolled), the second fluorescence measurement is taken.

This is followed by imposing a severe drought stress (SEV DRT) by withholding all water until the plants collapse. Duration of severe drought stress is 8-10 days and/or when plants have collapse. Thereafter, a recovery (REC) is imposed by watering all plants to 100% FC. Maintain 100% FC 72 hours. Survival score (yes/no) is recorded after 24, 48 and 72 hour recovery.

The entire shoot (Fresh) is sampled and weights are recorded (Fresh shoot weights). Fresh shoot material is then dried for 120 hrs at 70 degrees at which time a Dry Shoot weight is recorded.

Measured variables are defined as follows:

The variable “Fv′/Fm′ no stress” is a measure of the optimum quantum yield (Fv′/Fm′) under optimal water conditions on the uppermost fully extended leaf (most often the third leaf) at the inflection point, in the leaf margin and avoiding the mid rib. Fv′/Fm′ provides an estimate of the maximum efficiency of PSII photochemistry at a given PPFD, which is the PSII operating efficiency if all the PSII centers were open (Q_{A} oxidized).

The variable “Fv′/Fm′ stress” is a measure of the optimum quantum yield (Fv′/Fm′) under water stressed conditions (25% field capacity). The measure is preceded by a moderate drought period where field capacity drops from 60% to 20%. At which time the field capacity is brought to 25% and the measure collected.

The variable “phiPSII_no stress” is a measure of Photosystem II (PSII) efficiency under optimal water conditions on the uppermost fully extended leaf (most often the third leaf) at the inflection point, in the leaf margin and avoiding the mid rib. The phiPSII value provides an estimate of the PSII operating efficiency, which estimates the efficiency at which light absorbed by PSII is used for Q_{A} reduction.

The variable “phiPSII_stress” is a measure of Photosystem II (PSII) efficiency under water stressed conditions (25% field capacity). The measure is preceded by a moderate drought period where field capacity drops from 60% to 20%. At which time the field capacity is brought to 25% and the measure collected.

### Example 19

### Yield Analysis of Maize Lines with the Arabidopsis Lead Gene

A recombinant DNA construct containing a validated Arabidopsis gene can be introduced into an elite maize inbred line either by direct transformation or introgression from a separately transformed line.

Transgenic plants, either inbred or hybrid, can undergo more vigorous field-based experiments to study yield enhancement and/or stability under well-watered and water-limiting conditions.

Subsequent yield analysis can be done to determine whether plants that contain the validated Arabidopsis lead gene have an improvement in yield performance under water-limiting conditions, when compared to the control plants that do not contain the validated Arabidopsis lead gene. Specifically, drought conditions can be imposed during the flowering and/or grain fill period for plants that contain the validated Arabidopsis lead gene and the control plants. Reduction in yield can be measured for both. Plants containing the validated Arabidopsis lead gene have less yield loss relative to the control plants, for example, at least 25%, at least 20%, at least 15%, at least 10% or at least 5% less yield loss.

The above method may be used to select transgenic plants with increased yield, under water-limiting conditions and/or well-watered conditions, when compared to a control plant not comprising said recombinant DNA construct. Plants containing the validated Arabidopsis lead gene may have increased yield, under water-limiting conditions and/or well-watered conditions, relative to the control plants, for example, at least 5%, at least 10%, at least 15%, at least 20% or at least 25% increased yield.

### Example 20A

### Preparation of Maize MATE-Efflux Polypeptide Lead Gene Expression Vector for Transformation of Maize

Clones cfp6n.pk010.h3, cfp1n.pk004,c4, cfp6n.pk009.n19, cfp5n.pk002.e2 and the sequence SEQ ID NO:36 encode maize MATE-efflux polypeptides designated “Zm-MATE-EP1”, “Zm-MATE-EP2”, “Zm-MATE-EP3”, “Zm-MATE-EP4” and “Zm-MATE-EP5”, respectively (SEC) ID NOS:19, 21, 23, 25 and 37).

A MultiSite GATEWAY® LR recombination reaction was performedbetween the following multiple entry clones:

1. PHP31948, containing Att L4::Zm Ubi promoter::Zm Ubi 5′UTR::Zm Ubi intron 1::AttR1;

2. PHP20234, containing AttR2::PIN II term::AttL3; and

3. PHP33735, containing AttL1::Zm-MATE-EP3::AttL2; and the destination vector PHP22655 containing AttR4::ccdB::Cm^{r}::AttR3, to create an expression vector PHP33743. The vector PHP33743 contains the following expression cassettes:

1. Zm ubiquitin promoter::moPAT::PinII terminator; a cassette expressing the PAT herbicide resistance gene used for selection during the transformation process;

2. LTP2 promoter::DS-RED2::PinII terminator; a cassette expressing the DS-RED color marker gene used for seed sorting; and

3. AttB4:: Zm ubiquitin promoter::Att B1::Zm-MATE-EP3::AttB2::PinII terminator::AttB3; a cassette overexpressing the gene of interest, Zea mays MATE-efflux polypeptide-3.

### Example 20B

### Transformation of Maize with Maize MATE-EP Polypeptide Lead Genes Using Agrobacterium

The maize MATE-efflux polypeptide expression cassette present in vector PHP33743 can be introduced into a maize inbred line, or a transformable maize line derived from an elite maize inbred line, using Agrobacterium-mediated transformation as described in Examples 12 and 13.

Vector PHP33743 can be electroporated into the LBA4404 Agrobacterium strain containing vector PHP10523 (FIG. 7; SEQ ID NO:7) to create the co-integrate vector PHP33911. The co-integrate vector is formed by recombination of the 2 plasmids, PHP33743 and PHP10523, through the COS recombination sites contained on each vector. The co-integrate vector PHP33911 contains the same 3 expression cassettes as above (Example 20A) in addition to other genes (TET, TET, TRFA, ORI terminator, CTL, ORI V, VIR C1, VIR C2, VIR G, VIR B) needed for the Agrobacterium strain and the Agrobacterium-mediated transformation.

Analysis of Maize Lines Transformed with PHP33911 Encoding the Zm-MATE-EP3 Protein

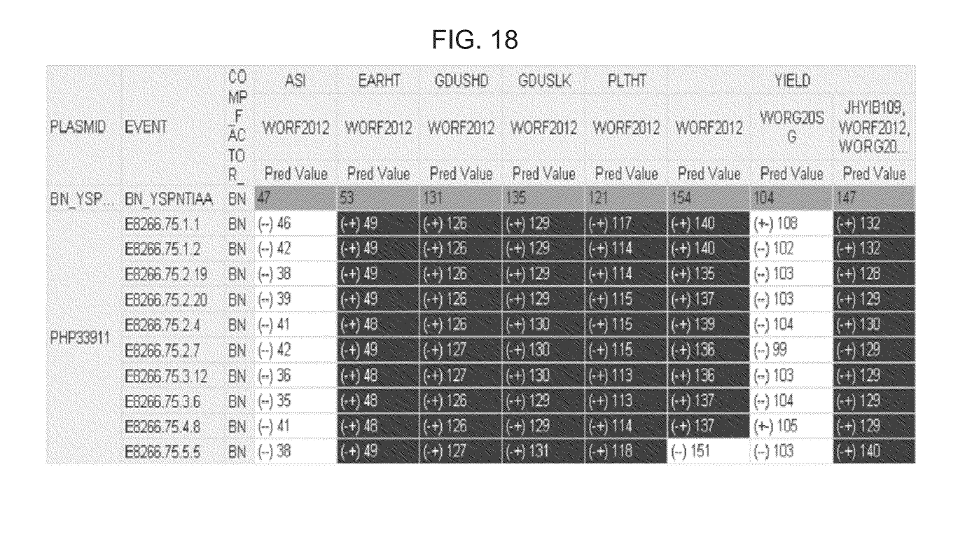

Agronomic data were collected in Woodland, Calif., in 2010 with 4-8 replicates per irrigation treatment. The WORF2012 location was subjected to a gradual drought treatment that reduced yield by about 35% compared to a well-watered field. Agronomic characteristics measured in this location included thermal time to anthesis and silking, and plant and ear height (inches), as well as grain yield (buiacre). The WORG20S location experienced a rapidly developing stress at flowering; this reduced yield by over 50%. Yield was measured at this location. Results for the 10 transgenic events are shown in FIG. 18 together with the bulk null control (BN).

Data analysis was by ASREML (VSN International Ltd), and the values are BLUPs (Best Linear Unbiased Prediction) (Cullis, B. R et al (1998) Biometrics 54: 1-18; Gilmour, A. R. et al (2009) ASRemI User Guide 3.0; Gilmour, A. R., et al (1995) Biometrics 51: 1440-50). For all traits, we performed single location analyses to calculate the BLUPs (Best Linear Unbiased Prediction) for each event; for yield, across-location analysis was conducted as well. The significance test between the event and BN was performed and the results are shown in FIG. 18.

As shown in FIG. 18, the effect of the transgene was significant and negative for thermal time to anthesis and silking, and the transgene also reduced both plant and ear height. The transgene reduced yield in all events with gradual stress, but this effect was not significant with the more severe, rapid stress. Minimal variation was detected among events. In the across-location analysis (last column in the table), all events yielded significantly less than the null.

### Example 21

### Preparation of Maize Expression Plasmids for Transformation into Gaspe Flint Derived Maize Lines

Clones cfp6n.pk010.h3, cfp1n.pk004.c4, cfp6n.pk009.n19 and cfp5n.pk002.e2 encode complete maize MATE-efflux polypeptides designated “Zm-MATE-EP1”, “Zm-MATE-EP2”, “Zm-MATE-EP3” and “Zm-MATE-EP4”, respectively (SEQ ID NOS:19, 21, 23 and 25)

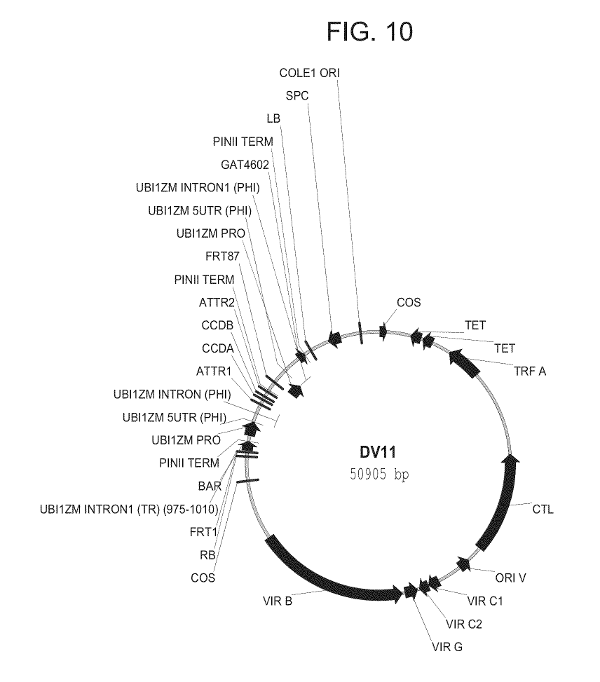

Using the INVITROGEN™ GATEWAY® Recombination technology, these clones encoding maize MATE-efflux polypeptide homologs were directionally cloned into the destination vector PHP29634 (SEQ ID NO:16; FIG. 10 to create the expression vectors listed in Table 6. Destination vector PHP29634 is similar to destination vector PHP23236; however, destination vector PHP29634 has site-specific recombination sites FRT1 and FRT87 and also encodes the GAT4602 selectable marker protein for selection of transformants using glyphosate. Each expression vector contains the cDNA of interest, encoding the Zea mays MATE-efflux polypeptides, under control of the UBI promoter and is a T-DNA binary vector for Agrobacterium-mediated transformation into corn as described, but not limited to, the examples described herein.

TABLE 6
Maize MATE-Efflux Polypeptide Expression Vectors

| | | SEQ ID NO: | Expression |
|---|---|---|---|
| Protein | Clone Origin | (Amino Acid) | Vector |
| ZmMATE-EP1 | cfp6n.pk010.h3 (FIS) | 20 | PHP33509 |
| ZmMATE-EP2 | cfp1n.pk004.c4 (FIS) | 22 | PHP33507 |
| ZmMATE-EP3 | cfp6n.pk009.n19 (FIS) | 24 | PHP33499 |
| Zm-MATE-EP4 | cfp5n.pk002.e2 (FIS) | 26 | PHP33459 |

### Example 22

### Transformation and Evaluation of Soybean with Soybean Homologs of Validated Lead Genes

Based on homology searches, one or several candidate soybean homologs of validated Arabidopsis lead genes can be identified and also be assessed for their ability to enhance drought tolerance in soybean. Vector construction, plant transformation and phenotypic analysis will be similar to that in previously described Examples.

### Example 23

### Transformation of Arabidopsis with Maize and Soybean Homologs of Validated Lead Genes

Soybean and maize homologs to validated Arabidopsis lead genes can be transformed into Arabidopsis under control of the 35S promoter and assessed for their ability to enhance drought tolerance in Arabidopsis. Vector construction, plant transformation and phenotypic analysis will be similar to that in previously described Examples.

### Example 24

### Evaluation of Arabidopsis and Maize MATE-EP Polypeptides by Expression Vectors Using Different Promoters

Recombinant constructs can be made to express MATE-EP polypeptides under different inducible or constitutive promoters. Inducible promoters include the following: drought inducible promoters (RAB18-At5g66400 and RD29A-At5g52310); heat inducible promoter (HSP; At5g12030); and root-specific promoters (PHT1; 1 (inorganic phosphate transporter 1-1)-At5g43350 and PIN2-At5g57090). Each of these constructs can be tested in different assays such as the drought, triple stress and osmotic stress assay.

### Example 25A

### Osmotic Stress Assay

To assay the osmotic stress tolerance of a transgenic line, a combination of osmolytes in the media, such as water soluble inorganic salts, sugar alcohols and high molecular weight non-penetrating osmolytes can be used to select for osmotically-tolerant plant lines.

The osmotic stress agents used in this assay are:

1) NaCl (sodium chloride)

2) Sorbitol

3) Mannitol

4) Polyethylene Glycol (PEG)

By providing these agents in the media, we aimed to mimic the multiple stress conditions in the in vitro environment thereby giving the plant the opportunity to respond to four stress agents.

### Methods and Materials:

The standardization of growth conditions and generation of kill curves for various osmotic stress agents individually was done before the development of quad stress assay conditions. Data generated from the kill curve experiments showed that the lethal concentrations for NaCl was 150 mM, sorbitol and mannitol was 500 mM, and PEG could only be used at 10% concentration (higher concentrations precipitated in the media). As there were four stress agents being used together, a quarter of each together in a solution would denote 100% stress or an osmotic pressure of 1.23 MPa. Therefore the following concentrations of each component are used in 100% quad media.

| Stress agents | Concentrations | |
|---|---|---|
| NaCl— | 62.5 | mM |
| Sorbitol- | 125 | mM |
| Mannitol- | 125 | mM |

| | |
|---|---|
| PEG- | 10% |

Assay Conditions: Seeds are surface sterilized and stratified for 48 hrs. About 100 seeds are inoculated in one plate and cultured in a growth chamber programmed for 16 h of light at 22° C. temperature and 50% relative humidity. Germination is scored as the emergence of radicle.
Assay Plan: A 6-day assay and an extended 10-day assay are done to test the seeds transgenic Arabidopsis line for osmotic stress tolerance.
Day 0—Surface sterilized seeds of different drought leads and stratify
Day 2—Inoculated onto quad media
Day 4—Counted for germination (48 hrs)
Day 5—Counted for germination (72 hrs) Take pictures or Scan plates from 48 hrs to 96 hrs.
Day 6—Counted for germination (96 hrs)
For the extended 10-day assay, germination is scored from 48 hrs to 96 hrs. On day 7, 8, 9 and 10, the emerged seedlings were checked for greenness and four leaf stage.

### Preparation of Media:

Germination medium (GM or 0%) for 1 liter:

| | | |
|---|---|---|
| MS salt | 4.3 | g |
| Sucrose | 10 | g |
| 1000x Vitamin mix | 1 | ml |
| MES (pH 5.7 with KOH) | 10 | ml |
| Phytagel (0.3%) | 3 | g |

To this the quad agents (the four osmolytes) are added by individually weighing the specific amounts in grams for their respective concentrations. Quad media preparation chart for all concentrations of osmolytes is given in Table 7.

TABLE 7

| | 10% | 20% | 30% | 40% | 50% | 60% | 70% | 80% | 90% | 100% |
|---|---|---|---|---|---|---|---|---|---|---|
| NaCl | 0.36 | 0.731 | 1.09 | 1.46 | 1.82 | 2.19 | 2.55 | 2.9 | 3.29 | 3.656 |
| Mannitol | 2.27 | 4.55 | 6.83 | 9.1 | 11.38 | 13.66 | 15.93 | 18.2 | 20.49 | 22.77 |
| Sorbitol | 2.27 | 4.55 | 6.83 | 9.1 | 11.38 | 13.66 | 15.93 | 18.2 | 20.49 | 22.77 |
| PEG | 10 | 20 | 30 | 40 | 50 | 60 | 70 | 80 | 90 | 100 |

### Sterilization of Seeds:

Approximately 100 μl of Arabidopsis Columbia wild type seeds (col wt) and the seeds of the transgenic line to be tested are taken in 1.75 ml microfuge tubes and sterilized in ethanol for 1 min 30 sec followed by one wash with sterile water. Then they are subjected to bleach treatment (4% bleach with Tween 20) for 2 min 30 sec. This is followed by 4 to 5 washes in sterile water. Seeds are stratified at 4° C. for 48 hrs before inoculation.

### Inoculation of Seeds:

Stratified seeds are plated onto a single plate of each quad stress concentration as given in Table 7. Plates are cultured in the chambers set at 16 h of light at 22° C. temperature and 50% relative humidity. Germination is scored as the emergence of radicle over a period of 48 to 96 hrs. Seeds are counted manually using a magnifying lens. Plates are scanned at 800 dpi using Epson scanner 10,000 XL and photographed. In case of the extended assay, leaf greenness (manual) and true leaf emergence i.e, 4Leaf stage (manual scoring) are also scored over a period of 10 days to account for the growth rate and health of the germinated seedlings.

The data is analyzed as percentage germination to the total number of seeds that are inoculated. Analyzed data is represented in the form of bar graphs and sigmoid curves by plotting quad concentrations against percent germination.

### Example 25B

### Seedling Emergence under Osmotic Stress of Transgenic Arabidopsis Seeds with At-MATE-EP Proteins

T1 seeds from transgenic Arabidopsis line with At-MATE-EP protein, containing the 35S promoter::At2g04090 expression construct pBC-Yellow-At2g04090, generated as described in Example 5, were screened for seedling emergence under osmotic stress as described in Example 24A.

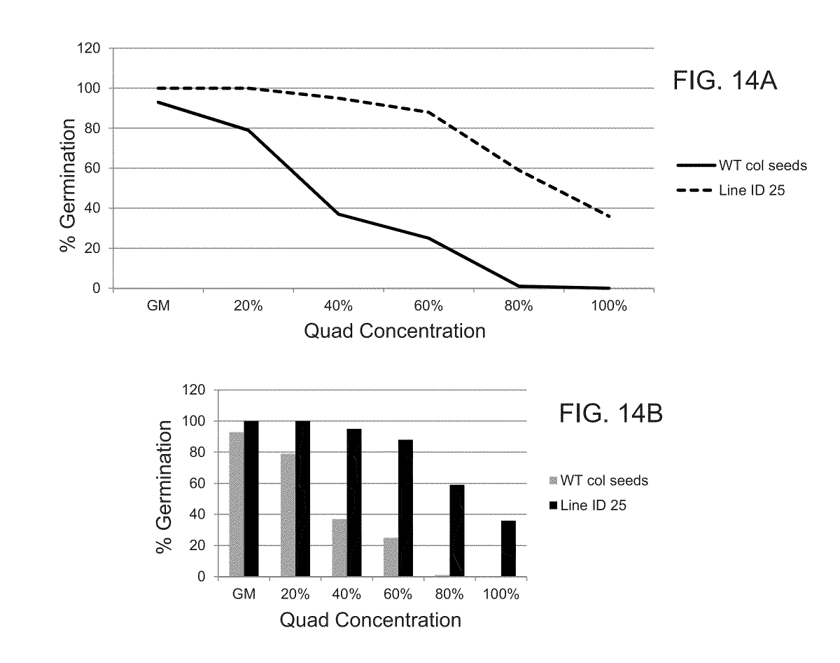

Arabidopsis Columbia seeds were used as wild-type control and at 60% there was a dip in germination and thereafter a decline and zero germination at 100%, as shown in FIG. 14A, FIG. 14B and Table 8.

Table 8 presents the percentage germination data at 48 hours for seedling emergence under osmotic stress.

TABLE 8

| | WT | Line ID 25 |
|---|---|---|
| GM | 93 | 100 |
| 20% | 79 | 100 |
| 40% | 37 | 95 |
| 60% | 25 | 88 |
| 80% | 1 | 59 |
| 100% | 0 | 36 |

### Seedling Emergence Under Osmotic Stress—10 Day Assay:

The results in FIG. 14A and FIG. 14B demonstrate that the transgenic Arabidopsis line containing the 35 S promoter::At2g04090 expression construct, pBC-Yellow-At2g04090, which was previously selected as having a drought tolerance phenotype, also demonstrates increased seedling emergence under osmotic stress.

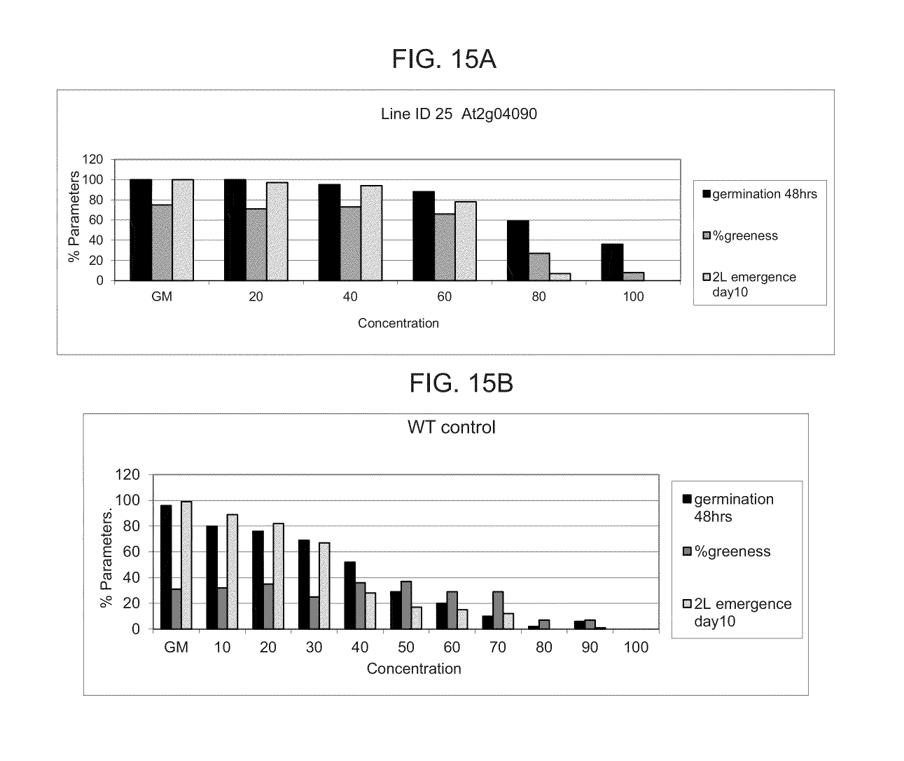

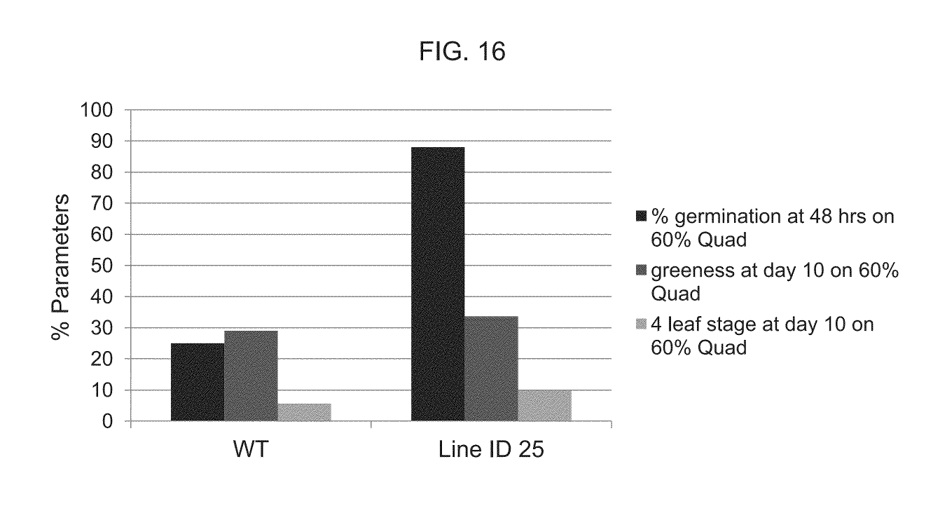

The osmotic stress assay for Line ID 25 was repeated, and scored for percentage greenness and percentage leaf emergence in an extended 10 day assay as well. The line was scored at 60% quad, for germination at 48 hours, and for percentage greenness and percentage leaf emergence in an extended 10 day assay. The results are shown in FIG. 15A, FIG. 15B, FIG. 16 and Table 10.

Percentage greenness and percentage leaf emergence were assayed. Percentage greenness was scored as the percentage of seedlings with green leaves (cotyledonary or true leaves) compared to yellow, brown or purple leaves. Greenness was scored manually and if there was any yellow or brown streaks on any of the 4 leaves, it was not considered green. Greenness was counted for seedlings with total green leaves only.

The leaf emergence was scored as the appearance of fully expanded leaves 1 and 2, after the two cotyledonary leaves had fully expanded. Therefore, the percentage leaf emergence is the number of seedlings with 2 true leaves or 4 leaves in total (2 cotyledonary and 2 true leaves).

TABLE 9
Percentage Parameters (Germination, Greenness,
and Leaf Emergence) for Wild-Type Plants

| | % Germination | % Greenness | 2L Emergence |
|---|---|---|---|
| WT | at 48 hrs | on Day 10 | on Day 10 |
| GM | 96 | 31 | 99 |
| 10 | 80 | 32 | 89 |
| 20 | 76 | 35 | 82 |
| 30 | 69 | 25 | 67 |
| 40 | 52 | 36 | 28 |
| 50 | 29 | 37 | 17 |
| 60 | 20 | 29 | 15 |
| 70 | 10 | 29 | 12 |
| 80 | 2 | 7 | 0 |
| 90 | 6 | 7 | 1 |
| 100 | 0 | 0 | 0 |

TABLE 10
Percentage Parameters (Germination, Greenness, and Leaf
Emergence) for At2g04090 Transgenic Plants (Line ID 25)

| LINE | % Germination | | 2L Emergence |
|---|---|---|---|
| ID 25 | at 48 hrs | % Greenness | on Day 10 |
| GM | 100 | 75 | 100 |
| 20 | 100 | 71 | 97 |
| 40 | 95 | 73 | 94 |
| 60 | 88 | 66 | 78 |
| 80 | 59 | 27 | 7 |
| 100 | 36 | 8 | 0 |

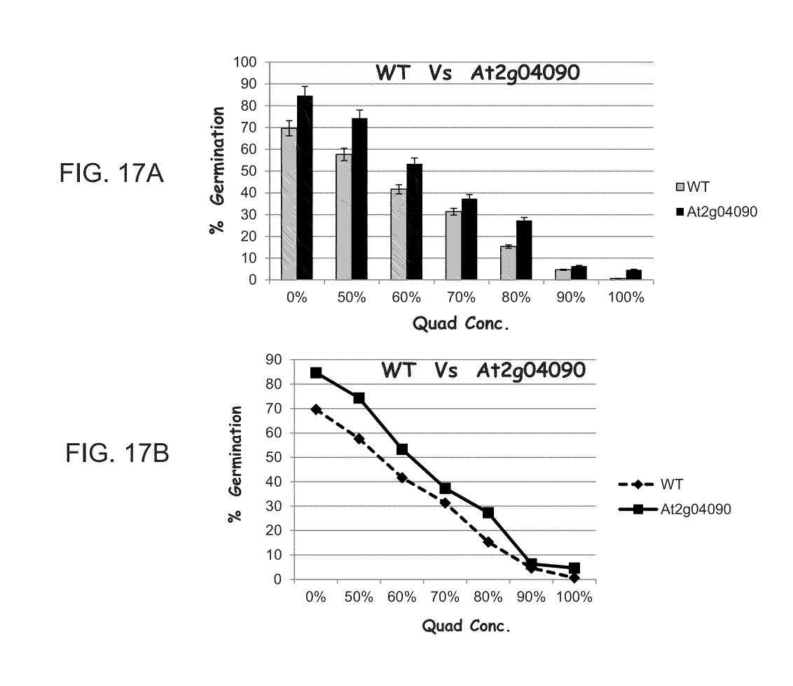

The percentage germination experiment at 48 hours was repeated once more with bulked seeds, in triplicates, and the results are shown in FIG. 17A, FIG. 17B and Table 11. Seeds were plated on MSO plate containing MS media+methionine sulphoximine and selected plants transplanted to the soil, seeds harvested and assayed.

TABLE 11

| | WT | At2g04090 |
|---|---|---|
| 0% | 70 | 85 |
| 50% | 58 | 74 |
| 60% | 42 | 53 |
| 70% | 31 | 37 |
| 80% | 15 | 27 |
| 90% | 5 | 6 |
| 100% | 1 | 5 |

1. A plant comprising in its genome a recombinant DNA construct comprising a polynucleotide operably linked to at least one regulatory element, wherein said polynucleotide encodes a polypeptide having an amino acid sequence of at least 80% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102 and wherein said plant exhibits an increase in at least one trait selected from the group consisting of: drought tolerance, yield, biomass and tolerance to osmotic stress, when compared to a control plant not comprising said recombinant DNA construct.

2. (canceled)

3. (canceled)

4. The plant of claim 1, wherein said plant exhibits said increase in yield, biomass, or both, when compared, under water limiting conditions, to said control plant not comprising said recombinant DNA construct.

5. The plant of claim 1, wherein said plant is selected from the group consisting of: Arabidopsis, maize, soybean, sunflower, sorghum, canola, wheat, alfalfa, cotton, rice, barley, millet, sugar cane and switchgrass.

6. Seed of the plant of claim 1, wherein said seed comprises in its genome a recombinant DNA construct comprising a polynucleotide operably linked to at least one regulatory element, wherein said polynucleotide encodes a polypeptide having an amino acid sequence of at least 80% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102 and wherein a plant produced from said seed exhibits an increase in at least one trait selected from the group consisting of: drought tolerance, osmotic stress tolerance, yield and biomass, when compared to a control plant not comprising said recombinant DNA construct.

7. (canceled)

8. A method of selecting for an increase in a plant of at least one trait selected from the group consisting of: drought tolerance, yield and biomass, comprising:
(a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 80% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102;
(b) obtaining a progeny plant derived from the transgenic plant of (a), wherein the progeny plant comprises in its genome the recombinant DNA construct; and
(c) selecting the progeny plant of (b) with an increase in at least one trait selected from the group consisting of: drought tolerance, yield and biomass, when compared to a control plant not comprising the recombinant DNA construct.

9. (canceled)

10. The method of claim 8, wherein said selecting step (c) comprises selecting the progeny plant of (b) that exhibits an increase of yield, biomass or both when compared, under water limiting conditions, to a control plant not comprising the recombinant DNA construct.

11. (canceled)

12. (canceled)

13. A method of increasing abiotic stress tolerance in a plant, comprising:
(a) introducing into a regenerable plant cell a recombinant DNA construct comprising a polynucleotide operably linked to at least one regulatory sequence, wherein the polynucleotide encodes a polypeptide having an amino acid sequence of at least 80% sequence identity, based on the Clustal V method of alignment, when compared to SEQ ID NOS: 18, 20, 22, 24, 26, 30, 31, 35, 37, 38, 39, 41-49, 51-65, 69, 71, 73, 75, 77, 79, 81, 83, 85, 87, 88-101 or 102;
(b) regenerating a transgenic plant from the regenerable plant cell after step (a), wherein the transgenic plant comprises in its genome the recombinant DNA construct; and
(c) obtaining a progeny plant derived from the transgenic plant of step (b), wherein said progeny plant comprises in its genome the recombinant DNA construct and exhibits increased tolerance at least one abiotic stress selected from the group consisting of: drought stress, osmotic stress, heat stress, high light stress, salt stress, paraquat stress and cold temperature stress, when compared to a control plant not comprising the recombinant DNA construct.

14. The method of claim 8, wherein said plant is selected from the group consisting of: Arabidopsis, maize, soybean, sunflower, sorghum, canola, wheat, alfalfa, cotton, rice, barley, millet, sugar cane and switchgrass.

15. An isolated polynucleotide comprising:
(a) a nucleotide sequence encoding a polypeptide with drought tolerance activity, wherein, based on the Clustal V method of alignment with pairwise alignment default parameters of KTUPLE=1, GAP PENALTY=3, WINDOW=5 and DIAGONALS SAVED=5, the polypeptide has an amino acid sequence of at least 95% sequence identity when compared to SEQ ID NO:18, 20, 22, 24, 26, 30, 31, 35, 37, 38 or 39; or
(b) the full complement of the nucleotide sequence of (a).

16. The polynucleotide of claim 15, wherein the amino acid sequence of the polypeptide comprises SEQ ID NO:18, 20, 22, 24, 26, 30, 31, 35, 37, 38, 39, 41-49, 51-65, 69, 71, 73, 75, 77, 79, 81, 83, 85, 87, 88-101 or 102.

17. The polynucleotide of claim 15 wherein the nucleotide sequence comprises SEQ ID NO:17, 19, 21, 23, 25, 36, 50, 66, 68, 70, 72, 74, 76, 78, 80, 82, 84 or 86.

18. A plant or seed comprising a recombinant DNA construct, wherein the recombinant DNA construct comprises the polynucleotide of claim 15 operably linked to at least one regulatory sequence.

19. A plant or seed comprising a recombinant DNA construct, wherein the recombinant DNA construct comprises the polynucleotide of claim 16 operably linked to at least one regulatory sequence.

20. A plant or seed comprising a recombinant DNA construct, wherein the recombinant DNA construct comprises the polynucleotide of claim 17 operably linked to at least one regulatory sequence.

21. Seed of the plant of claim 5, wherein said seed comprises in its genome a recombinant DNA construct comprising a polynucleotide operably linked to at least one regulatory element, wherein said polynucleotide encodes a polypeptide having an amino acid sequence of at least 80% sequence identity, based on the Clustal V method of alignment, when compared to SEQ ID NO:18, 20, 22, 24, 26, 30, 31, 35, 37, 38, 39, 41-49, 51-65, 69, 71, 73, 75, 77, 79, 81, 83, 85, 87, 88-101 or 102 and wherein a plant produced from said seed exhibits an increase in at least one trait selected from the group consisting of: drought tolerance, osmotic stress tolerance, yield and biomass, when compared to a control plant not comprising said recombinant DNA construct.

22. The method of claim 10, wherein said plant is selected from the group consisting of: Arabidopsis, maize, soybean, sunflower, sorghum, canola, wheat, alfalfa, cotton, rice, barley, millet, sugar cane and switchgrass.

23. The method of claim 13, wherein said plant is selected from the group consisting of: Arabidopsis, maize, soybean, sunflower, sorghum, canola, wheat, alfalfa, cotton, rice, barley, millet, sugar cane and switchgrass.


SEQUENCE LISTING (103 sequences)

SEQ ID NO: 1 - DNA, 18491 bp, artificial sequence
  pHSbarENDs2 activation tagging construct
catgaatcaaacaaacatacacagcgacttattcacacgagctcaaattacaacggtatatatcctgccgtcgacaaccatggtctagacaggatccccgggtaccgagctcgaatttgcaggtcgactgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaattgccctttggtcttctgagactgttgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcagttaacccgcgatcctgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaattgccctttggtcttctgagactgttgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcagttaacccgcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggatcgatccgtcgatcgaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgaacgttattagttcgccgctcggtgtgtcgtagatactagcccctggggccttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgccccccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttctagggggggggtaccgatctgagatcggtaacgaaaacgaacgggtagggatgaaaacggtcggtaacggtcggtaaaatacctctaccgttttcattttcatatttaacttgcgggacggaaacgaaaacgggatataccggtaacgaaaacgaacgggataaatacggtaatcgaaaaccgatacgatccggtcgggttaaagtcgaaatcggacgggaaccggtatttttgttcggtaaaatcacacatgaaaacatatattcaaaacttaaaaacaaatataaaaaattgtaaacacaagtcttaatgatcactagtggcgcgcctaggagatctcgagtagggataacagggtaatacatagataaaatccatataaatctggagcacacatagtttaatgtagcacataagtgataagtcttgggctcttggctaacataagaagccatataagtctactagcacacatgacacaatataaagtttaaaacacatattcataatcacttgctcacatctggatcacttagcatgctacagctagtgcaatattagacactttccaatatttctcaaacttttcactcattgcaacggccattctcctaatgacaaatttttcatgaacacaccattggtcaatcaaatcctttatctcacagaaacctttgtaaaataaatttgcagtggaatattgagtaccagataggagttcagtgagatcaaaaaacttcttcaaacacttaaaaagagttaatgccatcttccactcctcggctttaggacaaattgcatcgtacctacaataattgacatttgattaattgagaatttataatgatgacatgtacaacaattgagacaaacatacctgcgaggatcacttgttttaagccgtgttagtgcaggcttataatataaggcatccctcaacatcaaataggttgaattccatctagttgagacatcatatgagatccctttagatttatccaagtcacattcactagcacacttcattagttcttcccactgcaaaggagaagattttacagcaagaacaatcgctttgattttctcaattgttcctgcaattacagccaagccatcctttgcaaccaagttcagtatgtgacaagcacacctcacatgaaagaaagcaccatcacaaactagatttgaatcagtgtcctgcaaatcctcaattatatcgtgcacagctacttcatttgcactagcattatccaaagacaaggcaaacaattttttctcaatgttccacttaaccatgattgcagtgaaggtttgtgataacctttggccagtgtggcgcccttcaacatgaaaaaagccaacaattcttttttggagacaccaatcatcatcaatccaatggatggtgacacacatgtatgacttattttgacaagatgtccacatatccatagttgtactgaagcgagactgaacatcttttagttttccatacaacttttctttttcttccaaatacaaatccatgatatattttctagcagtgacacgggactttattggaaagtgagggcgcagagacttaacaaactcaacaaagtactcatgttctacaatattgaaaggatattcatgcatgattattgccaaatgaagcttctttaggctaaccacttcatcgtacttataaggctcaatgagatttatgtctttgccatgatccttttcactttttagacacaactgacctttaactaaactatgtgatgttctcaagtgatttcgaaatccgcttgttccatgatgaccctcagccctatacttagccttgcaattaggaaagttgcaatgtccccatacctgaacgtatttctttccatcgacctccacttcaatttccttcttggtgaaatgctgccatacatccgatgtgcacttctttgccctcttctgtggtgcttcttcttcgggttcaggttgtggctgtggttgtggttctggttgtggttgtggttgtggttgtggttcatgaacaatagccatatcatcttgactcggatctgtagctgtaccatttgcattactactgcttacactctgaataaaatgcctctcggcctcagctgttgatgatgatggtgatgtgcggccacatccatgcccacgcgcacgtgcacgtacattctgaatccgactagaagaggcttcagcttttcttttcaaccctgttataaacagatttttcgtattattctacagtcaatatgatgcttcccaatctacaaccaattagtaatgctaatgctattgctactgtttttctaatatataccttgagcatatgcagagaatacggaatttgttttgcgagtagaaggcgctcttgtggtagacatcaacttggccaatcttatggctgagcctgagggaggattatttccaaccggaggcgtcatctgaggaatggagtcgtagccggctagccgaagtggagagcagagccctggacagcaggtgttcagcaatcagcttggtgctgtactgctgtgacttgtgagcacctggacggctggacagcaatcagcaggtgttgcagagcccctggacagcacacaaatgacacaacagcttggtgcaatggtgctgacgtgctgtactgctaagtgctgtgagcctgtgagcagccgtggagacagggagaccgcggatggccggatgggcgagcgccgagcagtggaggtctggaggaccgctgaccgcagatggcggatggcggatgggcggaccgcggatgggcgagcagtggagtggaggtctgggcggatgggcggaccgcggcgcggatgggcgagtcgcgagcagtggagtggagggcggaccgtggatggcggcgtctgcgtccggcgtgccgcgtcacggccgtcaccgcgtgtggtgcctggtgcagcccagcggccggccggctgggagacagggagagtcggagagagcaggcgagagcgagacgcgtcgccggcgtcggcgtgcggctggcggcgtccggactccggcgtgggcgcgtggcggcgtgtgaatgtgtgatgctgttactcgtgtggtgcctggccgcctgggagagaggcagagcagcgttcgctaggtatttcttacatgggctgggcctcagtggttatggatgggagttggagctggccatattgcagtcatcccgaattagaaaatacggtaacgaaacgggatcatcccgattaaaaacgggatcccggtgaaacggtcgggaaactagctctaccgtttccgtttccgtttaccgttttgtatatcccgtttccgttccgttttcgttttttacctcgggttcgaaatcgatcgggataaaactaacaaaatcggttatacgataacggtcggtacgggattttcccatcctactttcatccctgagattattgtcgtttctttcgcagatcggtaccccccccctagagtcgacatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttaggtacggactagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaatcgttgcggttctgtcagttccaaacgtaaaacggcttgtcccgcgtcatcggcgggggtcataacgtgactcccttaattctccgctcatgatccccgggtaccgagctcgaattgcggctgagtggctccttcaatcgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtatcgatatggtggatttatcacaaatgggacccgccgccgacagaggtgtgatgttaggccaggactttgaaaatttgcgcaactatcgtatagtggccgacaaattgacgccgagttgacagactgcctagcatttgagtgaattatgtgaggtaatgggctacactgaattggtagctcaaactgtcagtatttatgtatatgagtgtatattttcgcataatctcagaccaatctgaagatgaaatgggtatctgggaatggcgaaatcaaggcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggtccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaagggaacgcatgaagttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaagacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcaattcggtcgatatggctattacgaagaaggctcgtgcgcggagtcccgtgaactttcccacgcaacaagtgaaccgcaccgggtttgccggaggccatttcgttaaaatgcgcagc

SEQ ID NO: 2 - DNA, 4291 bp, artificial sequence
  Gateway donor vector pDONR-Zeo
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacacattgatgagcaatgcttttttataatgccaactttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccacgcggcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaataatatcatcatgatcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 3 - DNA, 4762 bp, Artificial Sequence
  gateway donor vector pDONR221
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacacattgatgagcaatgcttttttataatgccaactttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccacgcggcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 4 - DNA, 16843 bp, artificial sequence
  destination vector for use with arabidopsis
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacctgtatggccgcattcgcaaaacacacctagactagatttgttttgctaacccaattgatattaattatatatgattaatatttatatgtatatggatttggttaatgaaatgcatctggttcatcaaagaattataaagacacgtgacattcatttaggataagaaatatggatgatctctttctcttttattcagataactagtaattacacataacacacaactttgatgcccacattatagtgattagcatgtcactatgtgtgcatccttttatttcatacattaattaagttggccaatccagaagatggacaagtctaggttaaccatgtggtacctacgcgttcgaatatccatgggccgcttcaggccagggcgctggggaaggcgatggcgtgctcggtcagctgccacttctggttcttggcgtcgctccggtcctcccgcagcagcttgtgctggatgaagtgccactcgggcatcttgctgggcacgctcttggccttgtacacggtgtcgaactggcaccggtaccggccgccgtccttcagcagcaggtacatgctcacgtcgcccttcaggatgccctgcttaggcacgggcatgatcttctcgcagctggcctcccagttggtggtcatcttcttcatcacggggccgtcggcggggaagttcacgccgttgaagatgctcttgtggtagatgcagttctccttcacgctcacggtgatgtccacgttacagatgcacacggcgccgtcctcgaacaggaagctccggccccaggtgtagccggcggggcagctgttcttgaagtagtccacgatgtcctgggggtactcggtgaagatccggtcgccgtacttgaagccggcgctcaggatgtcctcgctgaagggcagggggccgccctcgatcacgcacaggttgatggtctgcttgcccttgaaggggtagccgatgccctcgccggtgatcacgaacttgtggccgttcacgcagccctccatgtggtacttcatggtcatctcctccttcaggccgtgcttgctgtgggccatggtggcgaccggtgaattcgagctcggtacccggggatcctgagtaaaacagaggagggtctcactaagtttatagagagactgagagagataaagggacacgtatgaagcgtctgttttcgtggtgtgacgtcaaagtcattttgctctctacgcgtgtctgtgtcggcttgatctttttttttgctttttggaactcatgtcggtagtatatcttttatttattttttctttttttcccttttctttcaaactgatgtcggtatgatatttattccatcctaaaatgtaacttactattattagtagtcggtccatgtctattggcccatcatgtggtcattttacgtttacgtcgtgtggctgtttattataacaaacggcacatccttctcattcgaattgtatttctccttaatcgttctaataggtatgatcttttattttatacgtaaaattaaaattgaatgatgtcaagaacgaaaattaatttgtatttacaaaggagctaaatattgtttattcctctactggtagaagataaaagaagtagatgaaataatgatcttactagagaatattcctcatttacactagtcaaatggaaatcttgtaaacttttacaataatttatcctgaaaatatgaaaaaatagaagaaaatgtttacctcctctctcctcttaattcacctacgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttgttgaaacatccctgaagtgtctcattttattttatttattctttgctgataaaaaaataaaataaaagaagctaagcacacggtcaaccattgctctactgctaaaagggttatgtgtagtgttttactgcataaattatgcagcaaacaagacaactcaaattaaaaaatttcctttgcttgtttttttgttgtctctgacttgactttcttgtggaagttggttgtataaggattgggacacaccattgtccttcttaatttaattttatttctttgctgataaaaaaaaaaaatttcatatagtgttaaataataatttgttaaataaccaaaaagtcaaatatgtttactctcgtttaaataattgagagtcgtccagcaaggctaaacgattgtatagatttatgacaatatttacttttttatagataaatgttatattataataaatttatatacatatattatatgttatttattatttattattattttaaatccttcaatattttatcaaaccaactcataattttttttttatctgtaagaagcaataaaattaaatagacccactttaaggatgatccaacctttatacagagtaagagagttcaaatagtaccctttcatatacatatcaactaaaatattagaaatatcatggatcaaaccttataaagacattaaataagtggataagtataatatataaatgggtagtatataatatataaatggatacaaacttctctctttataattgttatgtctccttaacatcctaatataatacataagtgggtaatatataatatataaatggagacaaacttcttccattataattgttatgtcttcttaacacttatgtctcgttcacaatgctaaagttagaattgtttagaaagtcttatagtacacatttgtttttgtactatttgaagcattccataagccgtcacgattcagatgatttataataataagaggaaatttatcatagaacaataaggtgcatagatagagtgttaatatatcataacatcctttgtttattcatagaagaagtgagatggagctcagttattatactgttacatggtcggatacaatattccatgctctccatgagctcttacacctacatgcattttagttcatacttcatgcacgtggccatcacagctagctgcagctacatatttacattttacaacaccaggagaactgccctgttagtgcataacaatcagaagatggccgtggctactcgagttatcgaaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatcctctagattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgtttgataaccggtactagtgtgcacgtcgagcgtgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttggtcttctgagactgtatctttgacatttttggagtagaccagagtgtcgtgctccaccatgttgacgaagattttcttcttgtcattgagtcgtaaaagactctgtatgaactgttcgccagtcttcacggcgagttctgttagatcctcgatttgaatcttagactccatgcatggccttagattcagtaggaactacctttttagagactccaatctctattacttgccttggtttatgaagcaagccttgaatcgtccatactggaatagtacttctgatcttgagaaatatgtctttctctgtgttcttgatgcaattagtcctgaatcttttgactgcatctttaaccttcttgggaaggtatttgatctcctggagattgttactcgggtagatcgtcttgatgagacctgctgcgtaggcctctctaaccatctgtgggtcagcattctttctgaaattgaagaggctaaccttctcattatcagtggtgaacatagtgtcgtcaccttcaccttcgaacttccttcctagatcgtaaagatagaggaaatcgtccattgtaatctccggggcaaaggagatctcttttggggctggatcactgctgggccttttggttcctagcgtgagccagtgggctttttgctttggtgggcttgttagggccttagcaaagctcttgggcttgagttgagcttctcctttggggatgaagttcaacctgtctgtttgctgacttgttgtgtacgcgtcagctgctgctcttgcctctgtaatagtggcaaatttcttgtgtgcaactccgggaacgccgtttgttgccgcctttgtacaaccccagtcatcgtatataccggcatgtggaccgttatacacaacgtagtagttgatatgagggtgttgaatacccgattctgctctgagaggagcaactgtgctgttaagctcagatttttgtgggattggaattggatcctctagagcaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttaggtacggactagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 5 - DNA, 9142 bp, artificial sequence
  destination vector for use with soybean
ctagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttgatgggtaccggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagctgggtctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgaatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtcaaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgacctgcagcccctccaccgcggtggcggccgctctagagatccgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatgatcctatgcgtatggtatgacgtgtgttcaagatgatgacttcaaacctacctatgacgtatggtatgacgtgtgtcgactgatgacttagatccactcgagcggctataaatacgtacctacgcaccctgcgctaccatccctagagctgcagcttatttttacaacaattaccaacaacaacaaacaacaaacaacattacaattactatttacaattacagtcgacccatcaacaagtttgtacaaaaaagctgaacgagaaacgtaaaatgatataaatatcaatatattaaattagattttgcataaaaaacagactacataatactgtaaaacacaacatatccagtcatattggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgataacctagacttgtccatcttctggattggccaacttaattaatgtatgaaataaaaggatgcacacatagtgacatgctaatcactataatgtgggcatcaaagttgtgtgttatgtgtaatta

SEQ ID NO: 6 - DNA, 49911 bp, artificial sequence
  destination vector for use with Gaspe-Flint derived maize lines
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatttaaatacgcgttgatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatatcattgacacgcccggtcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattccggtccgggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcggaccgattaaactttaattcggtccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttggtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacagtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcggagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaattgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgactctagagctcgttcctcgaggaacggtacctgcggggaagcttacaataatgtgtgttgttaagtcttgttgcctgtcatcgtctgactgactttcgtcataaatcccggcctccgtaacccagctttgggcaagctcacggatttgatccggcggaacgggaatatcgagatgccgggctgaacgctgcagttccagctttccctttcgggacaggtactccagctgattgattatctgctgaagggtcttggttccacctcctggcacaatgcgaatgattacttgagcgcgatcgggcatccaattttctcccgtcaggtgcgtggtcaagtgctacaaggcacctttcagtaacgagcgaccgtcgatccgtcgccgggatacggacaaaatggagcgcagtagtccatcgagggcggcgaaagcctcgccaaaagcaatacgttcatctcgcacagcctccagatccgatcgagggtcttcggcgtaggcagatagaagcatggatacattgcttgagagtattccgatggactgaagtatggcttccatcttttctcgtgtgtctgcatctatttcgagaaagcccccgatgcggcgcaccgcaacgcgaattgccatactatccgaaagtcccagcaggcgcgcttgataggaaaaggtttcatactcggccgatcgcagacgggcactcacgaccttgaacccttcaactttcagggatcgatgctggttgatggtagtctcactcgacgtggctctggtgtgttttgacatagcttcctccaaagaaagcggaaggtctggatactccagcacgaaatgtgcccgggtagacggatggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaatatgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtactgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttgaagatggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtgaagagggacctatcggaacccctcaccaaatattgagtgtaggtttgaggccgctggccgcgtcctcagtcaccttttgagccagataattaagagccaaatgcaattggctcaggctgccatcgtccccccgtgcgaaacctgcacgtccgcgtcaaagaaataaccggcacctcttgctgtttttatcagttgagggcttgacggatccgcctcaagtttgcggcgcagccgcaaaatgagaacatctatactcctgtcgtaaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgcaagaaactctctcgtactaaacggcttagcgataaaatcacttgctcctagctcgagtgcaacaactttatccgtctcctcaaggcggtcgccactgataattatgattggaatatcagactttgccgccagatttcgaacgatctcaagcccatcttcacgacctaaatttagatcaacaaccacgacatcgaccgtcgcggaagagagtactctagtgaactgggtgctgtcggctaccgcggtcactttgaaggcgtggatcgtaaggtattcgataataagatgccgcatagcgacatcgtcatcgataagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacgaaaatgcgaaccaagtatttcaattttatgacaaaagttctcaatcgttgttacaagtgaaacgcttcgaggttacagctactattgattaaggagatcgcctatggtctcgccccggcgtcgtgcgtccgccgcgagccagatctcgcctacttcataaacgtcctcataggcacggaatggaatgatgacatcgatcgccgtagagagcatgtcaatcagtgtgcgatcttccaagctagcaccttgggcgctacttttgacaagggaaaacagtttcttgaatccttggattggattcgcgccgtgtattgttgaaatcgatcccggatgtcccgagacgacttcactcagataagcccatgctgcatcgtcgcgcatctcgccaagcaatatccggtccggccgcatacgcagacttgcttggagcaagtgctcggcgctcacagcacccagcccagcaccgttcttggagtagagtagtctaacatgattatcgtgtggaatgacgagttcgagcgtatcttctatggtgattagcctttcctggggggggatggcgctgatcaaggtcttgctcattgttgtcttgccgcttccggtagggccacatagcaacatcgtcagtcggctgacgacgcatgcgtgcagaaacgcttccaaatccccgttgtcaaaatgctgaaggatagcttcatcatcctgattttggcgtttccttcgtgtctgccactggttccacctcgaagcatcataacgggaggagacttctttaagaccagaaacacgcgagcttggccgtcgaatggtcaagctgacggtgcccgagggaacggtcggcggcagacagatttgtagtcgttcaccaccaggaagttcagtggcgcagagggggttacgtggtccgacatcctgctttctcagcgcgcccgctaaaatagcgatatcttcaagatcatcataagagacgggcaaaggcatcttggtaaaaatgccggcttggcgcacaaatgcctctccaggtcgattgatcgcaatttcttcagtcttcgggtcatcgagccattccaaaatcggcttcagaagaaagcgtagttgcggatccacttccatttacaatgtatcctatctctaagcggaaatttgaattcattaagagcggcggttcctcccccgcgtggcgccgccagtcaggcggagctggtaaacaccaaagaaatcgaggtcccgtgctacgaaaatggaaacggtgtcaccctgattcttcttcagggttggcggtatgttgatggttgccttaagggctgtctcagttgtctgctcaccgttattttgaaagctgttgaagctcatcccgccacccgagctgccggcgtaggtgctagctgcctggaaggcgccttgaacaacactcaagagcatagctccgctaaaacgctgccagaagtggctgtcgaccgagcccggcaatcctgagcgaccgagttcgtccgcgcttggcgatgttaacgagatcatcgcatggtcaggtgtctcggcgcgatcccacaacacaaaaacgcgcccatctccctgttgcaagccacgctgtatttcgccaacaacggtggtgccacgatcaagaagcacgatattgttcgttgttccacgaatatcctgaggcaagacacactttacatagcctgccaaatttgtgtcgattgcggtttgcaagatgcacggaattattgtcccttgcgttaccataaaatcggggtgcggcaagagcgtggcgctgctgggctgcagctcggtgggtttcatacgtatcgacaaatcgttctcgccggacacttcgccattcggcaaggagttgtcgtcacgcttgccttcttgtcttcggcccgtgtcgccctgaatggcgcgtttgctgaccccttgatcgccgctgctatatgcaaaaatcggtgtttcttccggccgtggctcatgccgctccggttcgcccctcggcggtagaggagcagcaggctgaacagcctcttgaaccgctggaggatccggcggcacctcaatcggagctggatgaaatggcttggtgtttgttgcgatcaaagttgacggcgatgcgttctcattcaccttcttttggcgcccacctagccaaatgaggcttaatgataacgcgagaacgacacctccgacgatcaatttctgagaccccgaaagacgccggcgatgtttgtcggagaccagggatccagatgcatcaacctcatgtgccgcttgctgactatcgttattcatcccttcgcccccttcaggacgcgtttcacatcgggcctcaccgtgcccgtttgcggcctttggccaacgggatcgtaagcggtgttccagatacatagtactgtgtggccatccctcagacgccaacctcgggaaaccgaagaaatctcgacatcgctccctttaactgaatagttggcaacagcttccttgccatcaggattgatggtgtagatggagggtatgcgtacattgcccggaaagtggaataccgtcgtaaatccattgtcgaagacttcgagtggcaacagcgaacgatcgccttgggcgacgtagtgccaattactgtccgccgcaccaagggctgtgacaggctgatccaataaattctcagctttccgttgatattgtgcttccgcgtgtagtctgtccacaacagccttctgttgtgcctcccttcgccgagccgccgcatcgtcggcggggtaggcgaattggacgctgtaatagagatcgggctgctctttatcgaggtgggacagagtcttggaacttatactgaaaacataacggcgcatcccggagtcgcttgcggttagcacgattactggctgaggcgtgaggacctggcttgccttgaaaaatagataatttccccgcggtagggctgctagatctttgctatttgaaacggcaaccgctgtcaccgtttcgttcgtggcgaatgttacgaccaaagtagctccaaccgccgtcgagaggcgcaccacttgatcgggattgtaagccaaataacgcatgcgcggatctagcttgcccgccattggagtgtcttcagcctccgcaccagtcgcagcggcaaataaacatgctaaaatgaaaagtgcttttctgatcatggttcgctgtggcctacgtttgaaacggtatcttccgatgtctgataggaggtgacaaccagacctgccgggttggttagtctcaatctgccgggcaagctggtcaccttttcgtagcgaactgtcgcggtccacgtactcaccacaggcattttgccgtcaacgacgagggtccttttatagcgaatttgctgcgtgcttggagttacatcatttgaagcgatgtgctcgacctccaccctgccgcgtttgccaagaatgacttgaggcgaactgggattgggatagttgaagaattgctggtaatcctggcgcactgttggggcactgaagttcgataccaggtcgtaggcgtactgagcggtgtcggcatcataactctcgcgcaggcgaacgtactcccacaatgaggcgttaacgacggcctcctcttgagttgcaggcaatcgcgagacagacacctcgctgtcaacggtgccgtccggccgtatccatagatatacgggcacaagcctgctcaacggcaccattgtggctatagcgaacgcttgagcaacatttcccaaaatcgcgatagctgcgacagctgcaatgagtttggagagacgtcgcgccgatttcgctcgcgcggtttgaaaggcttctacttccttatagtgctcggcaaggctttcgcgcgccactagcatggcatattcaggccccgtcatagcgtccacccgaattgccgagctgaagatctgacggagtaggctgccatcgccccacattcagcgggaagatcgggcctttgcagctcgctaatgtgtcgtttgtctggcagccgctcaaagcgacaactaggcacagcaggcaatacttcatagaattctccattgaggcgaatttttgcgcgacctagcctcgctcaacctgagcgaagcgacggtacaagctgctggcagattgggttgcgccgctccagtaactgcctccaatgttgccggcgatcgccggcaaagcgacaatgagcgcatcccctgtcagaaaaaacatatcgagttcgtaaagaccaatgatcttggccgcggtcgtaccggcgaaggtgattacaccaagcataagggtgagcgcagtcgcttcggttaggatgacgatcgttgccacgaggtttaagaggagaagcaagagaccgtaggtgataagttgcccgatccacttagctgcgatgtcccgcgtgcgatcaaaaatatatccgacgaggatcagaggcccgatcgcgagaagcactttcgtgagaattccaacggcgtcgtaaactccgaaggcagaccagagcgtgccgtaaaggacccactgtgccccttggaaagcaaggatgtcctggtcgttcatcggaccgatttcggatgcgattttctgaaaaacggcctgggtcacggcgaacattgtatccaactgtgccggaacagtctgcagaggcaagccggttacactaaactgctgaacaaagtttgggaccgtcttttcgaagatggaaaccacatagtcttggtagttagcctgcccaacaattagagcaacaacgatggtgaccgtgatcacccgagtgataccgctacgggtatcgacttcgccgcgtatgactaaaataccctgaacaataatccaaagagtgacacaggcgatcaatggcgcactcaccgcctcctggatagtctcaagcatcgagtccaagcctgtcgtgaaggctacatcgaagatcgtatgaatggccgtaaacggcgccggaatcgtgaaattcatcgattggacctgaacttgactggtttgtcgcataatgttggataaaatgagctcgcattcggcgaggatgcgggcggatgaacaaatcgcccagccttaggggagggcaccaaagatgacagcggtcttttgatgctccttgcgttgagcggccgcctcttccgcctcgtgaaggccggcctgcgcggtagtcatcgttaataggcttgtcgcctgtacattttgaatcattgcgtcatggatctgcttgagaagcaaaccattggtcacggttgcctgcatgatattgcgagatcgggaaagctgagcagacgtatcagcattcgccgtcaagcgtttgtccatcgtttccagattgtcagccgcaatgccagcgctgtttgcggaaccggtgatctgcgatcgcaacaggtccgcttcagcatcactacccacgactgcacgatctgtatcgctggtgatcgcacgtgccgtggtcgacattggcattcgcggcgaaaacatttcattgtctaggtccttcgtcgaaggatactgatttttctggttgagcgaagtcagtagtccagtaacgccgtaggccgacgtcaacatcgtaaccatcgctatagtctgagtgagattctccgcagtcgcgagcgcagtcgcgagcgtctcagcctccgttgccgggtcgctaacaacaaactgcgcccgcgcgggctgaatatatagaaagctgcaggtcaaaactgttgcaataagttgcgtcgtcttcatcgtttcctaccttatcaatcttctgcctcgtggtgacgggccatgaattcgctgagccagccagatgagttgccttcttgtgcctcgcgtagtcgagttgcaaagcgcaccgtgttggcacgccccgaaagcacggcgacatattcacgcatatcccgcagatcaaattcgcagatgacgcttccactttctcgtttaagaagaaacttacggctgccgaccgtcatgtcttcacggatcgcctgaaattccttttcggtacatttcagtccatcgacataagccgatcgatctgcggttggtgatggatagaaaatcttcgtcatacattgcgcaaccaagctggctcctagcggcgattccagaacatgctctggttgctgcgttgccagtattagcatcccgttgttttttcgaacggtcaggaggaatttgtcgacgacagtcgaaaatttagggtttaacaaataggcgcgaaactcatcgcagctcatcacaaaacggcggccgtcgatcatggctccaatccgatgcaggagatatgctgcagcgggagcgcatacttcctcgtattcgagaagatgcgtcatgtcgaagccggtaatcgacggatctaactttacttcgtcaacttcgccgtcaaatgcccagccaagcgcatggccccggcaccagcgttggagccgcgctcctgcgccttcggcgggcccatgcaacaaaaattcacgtaaccccgcgattgaacgcatttgtggatcaaacgagagctgacgatggataccacggaccagacggcggttctcttccggagaaatcccaccccgaccatcactctcgatgagagccacgatccattcgcgcagaaaatcgtgtgaggctgctgtgttttctaggccacgcaacggcgccaacccgctgggtgtgcctctgtgaagtgccaaatatgttcctcctgtggcgcgaaccagcaattcgccaccccggtccttgtcaaagaacacgaccgtacctgcacggtcgaccatgctctgttcgagcatggctagaacaaacatcatgagcgtcgtcttacccctcccgataggcccgaatattgccgtcatgccaacatcgtgctcatgcgggatatagtcgaaaggcgttccgccattggtacgaaatcgggcaatcgcgttgccccagtggcctgagctggcgccctctggaaagttttcgaaagagacaaaccctgcgaaattgcgtgaagtgattgcgccagggcgtgtgcgccacttaaaattccccggcaattgggaccaataggccgcttccataccaataccttcttggacaaccacggcacctgcatccgccattcgtgtccgagcccgcgcgcccctgtccccaagactattgagatcgtctgcatagacgcaaaggctcaaatgatgtgagcccataacgaattcgttgctcgcaagtgcgtcctcagcctcggataatttgccgatttgagtcacggctttatcgccggaactcagcatctggctcgatttgaggctaagtttcgcgtgcgcttgcgggcgagtcaggaacgaaaaactctgcgtgagaacaagtggaaaatcgagggatagcagcgcgttgagcatgcccggccgtgtttttgcagggtattcgcgaaacgaatagatggatccaacgtaactgtcttttggcgttctgatctcgagtcctcgcttgccgcaaatgactctgtcggtataaatcgaagcgccgagtgagccgctgacgaccggaaccggtgtgaaccgaccagtcatgatcaaccgtagcgcttcgccaatttcggtgaagagcacaccctgcttctcgcggatgccaagacgatgcaggccatacgctttaagagagccagcgacaacatgccaaagatcttccatgttcctgatctggcccgtgagatcgttttccctttttccgcttagcttggtgaacctcctctttaccttccctaaagccgcctgtgggtagacaatcaacgtaaggaagtgttcattgcggaggagttggccggagagcacgcgctgttcaaaagcttcgttcaggctagcggcgaaaacactacggaagtgtcgcggcgccgatgatggcacgtcggcatgacgtacgaggtgagcatatattgacacatgatcatcagcgatattgcgcaacagcgtgttgaacgcacgacaacgcgcattgcgcatttcagtttcctcaagctcgaatgcaacgccatcaattctcgcaatggtcatgatcgatccgtcttcaagaaggacgatatggtcgctgaggtggccaatataagggagatagatctcaccggatctttcggtcgttccactcgcgccgagcatcacaccattcctctccctcgtgggggaaccctaattggatttgggctaacagtagcgcccccccaaactgcactatcaatgcttcttcccgcggtccgcaaaaatagcaggacgacgctcgccgcattgtagtctcgctccacgatgagccgggctgcaaaccataacggcacgagaacgacttcgtagagcgggttctgaacgataacgatgacaaagccggcgaacatcatgaataaccctgccaatgtcagtggcaccccaagaaacaatgcgggccgtgtggctgcgaggtaaagggtcgattcttccaaacgatcagccatcaactaccgccagtgagcgtttggccgaggaagctcgccccaaacatgataacaatgccgccgacgacgccggcaaccagcccaagcgaagcccgcccgaacatccaggagatcccgatagcgacaatgccgagaacagcgagtgactggccgaacggaccaaggataaacgtgcatatattgttaaccattgtggcggggtcagtgccgccacccgcagattgcgctgcggcgggtccggatgaggaaatgctccatgcaattgcaccgcacaagcttggggcgcagctcgatatcacgcgcatcatcgcattcgagagcgagaggcgatttagatgtaaacggtatctctcaaagcatcgcatcaatgcgcacctccttagtataagtcgaataagacttgattgtcgtctgcggatttgccgttgtcctggtgtggcggtggcggagcgattaaaccgccagcgccatcctcctgcgagcggcgctgatatgacccccaaacatcccacgtctcttcggattttagcgcctcgtgatcgtcttttggaggctcgattaacgcgggcaccagcgattgagcagctgtttcaacttttcgcacgtagccgtttgcaaaaccgccgatgaaattaccggtgttgtaagcggagatcgcccgacgaagcgcaaattgcttctcgtcaatcgtttcgccgcctgcataacgacttttcagcatgtttgcagcggcagataatgatgtgcacgcctggagcgcaccgtcaggtgtcagaccgagcatagaaaaatttcgagagtttatttgcatgaggccaacatccagcgaatgccgtgcatcgagacggtgcctgacgacttgggttgcttggctgtgatcttgccagtgaagcgtttcgccggtcgtgttgtcatgaatcgctaaaggatcaaagcgactctccaccttagctatcgccgcaagcgtagatgtcgcaactgatggggcacacttgcgagcaacatggtcaaactcagcagatgagagtggcgtggcaaggctcgacgaacagaaggagaccatcaaggcaagagaaagcgaccccgatctcttaagcataccttatctccttagctcgcaactaacaccgcctctcccgttggaagaagtgcgttgttttatgttgaagattatcgggagggtcggttactcgaaaattttcaattgcttctttatgatttcaattgaagcgagaaacctcgcccggcgtcttggaacgcaacatggaccgagaaccgcgcatccatgactaagcaaccggatcgacctattcaggccgcagttggtcaggtcaggctcagaacgaaaatgctcggcgaggttacgctgtctgtaaacccattcgatgaacgggaagcttccttccgattgctcttggcaggaatattggcccatgcctgcttgcgctttgcaaatgctcttatcgcgttggtatcatatgccttgtccgccagcagaaacgcactctaagcgattatttgtaaaaatgtttcggtcatgcggcggtcatgggcttgacccgctgtcagcgcaagacggatcggtcaaccgtcggcatcgacaacagcgtgaatcttggtggtcaaaccgccacgggaacgtcccatacagccatcgtcttgatcccgctgtttcccgtcgccgcatgttggtggacgcggacacaggaactgtcaatcatgacgacattctatcgaaagccttggaaatcacactcagaatatgatcccagacgtctgcctcacgccatcgtacaaagcgattgtagcaggttgtacaggaaccgtatcgatcaggaacgtctgcccagggcgggcccgtccggaagcgccacaagatgacattgatcacccgcgtcaacgcgcggcacgcgacgcggcttatttgggaacaaaggactgaacaacagtccattcgaaatcggtgacatcaaagcggggacgggttatcagtggcctccaagtcaagcctcaatgaatcaaaatcagaccgatttgcaaacctgatttatgagtgtgcggcctaaatgatgaaatcgtccttctagatcgcctccgtggtgtagcaacacctcgcagtatcgccgtgctgaccttggccagggaattgactggcaagggtgctttcacatgaccgctcttttggccgcgatagatgatttcgttgctgctttgggcacgtagaaggagagaagtcatatcggagaaattcctcctggcgcgagagcctgctctatcgcgacggcatcccactgtcgggaacagaccggatcattcacgaggcgaaagtcgtcaacacatgcgttataggcatcttcccttgaaggatgatcttgttgctgccaatctggaggtgcggcagccgcaggcagatgcgatctcagcgcaacttgcggcaaaacatctcactcacctgaaaaccactagcgagtctcgcgatcagacgaaggccttttacttaacgacacaatatccgatgtctgcatcacaggcgtcgctatcccagtcaatactaaagcggtgcaggaactaaagattactgatgacttaggcgtgccacgaggcctgagacgacgcgcgtagacagttttttgaaatcattatcaaagtgatggcctccgctgaagcctatcacctctgcgccggtctgtcggagagatgggcaagcattattacggtcttcgcgcccgtacatgcattggacgattgcagggtcaatggatctgagatcatccagaggattgccgcccttaccttccgtttcgagttggagccagcccctaaatgagacgacatagtcgacttgatgtgacaatgccaagagagagatttgcttaacccgatttttttgctcaagcgtaagcctattgaagcttgccggcatgacgtccgcgccgaaagaatatcctacaagtaaaacattctgcacaccgaaatgcttggtgtagacatcgattatgtgaccaagatccttagcagtttcgcttggggaccgctccgaccagaaataccgaagtgaactgacgccaatgacaggaatcccttccgtctgcagataggtaccatcgatagatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtagtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacccggaccgaagcttgcatgcctgcag

SEQ ID NO: 7 - DNA, 36909 bp, artificial sequence
  NCBI General Identifier No. 59797027
tctagagctcgttcctcgaggcctcgaggcctcgaggaacggtacctgcggggaagcttacaataatgtgtgttgttaagtcttgttgcctgtcatcgtctgactgactttcgtcataaatcccggcctccgtaacccagctttgggcaagctcacggatttgatccggcggaacgggaatatcgagatgccgggctgaacgctgcagttccagctttccctttcgggacaggtactccagctgattgattatctgctgaagggtcttggttccacctcctggcacaatgcgaatgattacttgagcgcgatcgggcatccaattttctcccgtcaggtgcgtggtcaagtgctacaaggcacctttcagtaacgagcgaccgtcgatccgtcgccgggatacggacaaaatggagcgcagtagtccatcgagggcggcgaaagcctcgccaaaagcaatacgttcatctcgcacagcctccagatccgatcgagggtcttcggcgtaggcagatagaagcatggatacattgcttgagagtattccgatggactgaagtatggcttccatcttttctcgtgtgtctgcatctatttcgagaaagcccccgatgcggcgcaccgcaacgcgaattgccatactatccgaaagtcccagcaggcgcgcttgataggaaaaggtttcatactcggccgatcgcagacgggcactcacgaccttgaacccttcaactttcagggatcgatgctggttgatggtagtctcactcgacgtggctctggtgtgttttgacatagcttcctccaaagaaagcggaaggtctggatactccagcacgaaatgtgcccgggtagacggatggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaatatgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtactgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttgaagatggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtgaagagggacctatcggaacccctcaccaaatattgagtgtaggtttgaggccgctggccgcgtcctcagtcaccttttgagccagataattaagagccaaatgcaattggctcaggctgccatcgtccccccgtgcgaaacctgcacgtccgcgtcaaagaaataaccggcacctcttgctgtttttatcagttgagggcttgacggatccgcctcaagtttgcggcgcagccgcaaaatgagaacatctatactcctgtcgtaaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgcaagaaactctctcgtactaaacggcttagcgataaaatcacttgctcctagctcgagtgcaacaactttatccgtctcctcaaggcggtcgccactgataattatgattggaatatcagactttgccgccagatttcgaacgatctcaagcccatcttcacgacctaaatttagatcaacaaccacgacatcgaccgtcgcggaagagagtactctagtgaactgggtgctgtcggctaccgcggtcactttgaaggcgtggatcgtaaggtattcgataataagatgccgcatagcgacatcgtcatcgataagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacgaaaatgcgaaccaagtatttcaattttatgacaaaagttctcaatcgttgttacaagtgaaacgcttcgaggttacagctactattgattaaggagatcgcctatggtctcgccccggcgtcgtgcgtccgccgcgagccagatctcgcctacttcataaacgtcctcataggcacggaatggaatgatgacatcgatcgccgtagagagcatgtcaatcagtgtgcgatcttccaagctagcaccttgggcgctacttttgacaagggaaaacagtttcttgaatccttggattggattcgcgccgtgtattgttgaaatcgatcccggatgtcccgagacgacttcactcagataagcccatgctgcatcgtcgcgcatctcgccaagcaatatccggtccggccgcatacgcagacttgcttggagcaagtgctcggcgctcacagcacccagcccagcaccgttcttggagtagagtagtctaacatgattatcgtgtggaatgacgagttcgagcgtatcttctatggtgattagcctttcctggggggggatggcgctgatcaaggtcttgctcattgttgtcttgccgcttccggtagggccacatagcaacatcgtcagtcggctgacgacgcatgcgtgcagaaacgcttccaaatccccgttgtcaaaatgctgaaggatagcttcatcatcctgattttggcgtttccttcgtgtctgccactggttccacctcgaagcatcataacgggaggagacttctttaagaccagaaacacgcgagcttggccgtcgaatggtcaagctgacggtgcccgagggaacggtcggcggcagacagatttgtagtcgttcaccaccaggaagttcagtggcgcagagggggttacgtggtccgacatcctgctttctcagcgcgcccgctaaaatagcgatatcttcaagatcatcataagagacgggcaaaggcatcttggtaaaaatgccggcttggcgcacaaatgcctctccaggtcgattgatcgcaatttcttcagtcttcgggtcatcgagccattccaaaatcggcttcagaagaaagcgtagttgcggatccacttccatttacaatgtatcctatctctaagcggaaatttgaattcattaagagcggcggttcctcccccgcgtggcgccgccagtcaggcggagctggtaaacaccaaagaaatcgaggtcccgtgctacgaaaatggaaacggtgtcaccctgattcttcttcagggttggcggtatgttgatggttgccttaagggctgtctcagttgtctgctcaccgttattttgaaagctgttgaagctcatcccgccacccgagctgccggcgtaggtgctagctgcctggaaggcgccttgaacaacactcaagagcatagctccgctaaaacgctgccagaagtggctgtcgaccgagcccggcaatcctgagcgaccgagttcgtccgcgcttggcgatgttaacgagatcatcgcatggtcaggtgtctcggcgcgatcccacaacacaaaaacgcgcccatctccctgttgcaagccacgctgtatttcgccaacaacggtggtgccacgatcaagaagcacgatattgttcgttgttccacgaatatcctgaggcaagacacactttacatagcctgccaaatttgtgtcgattgcggtttgcaagatgcacggaattattgtcccttgcgttaccataaaatcggggtgcggcaagagcgtggcgctgctgggctgcagctcggtgggtttcatacgtatcgacaaatcgttctcgccggacacttcgccattcggcaaggagttgtcgtcacgcttgccttcttgtcttcggcccgtgtcgccctgaatggcgcgtttgctgaccccttgatcgccgctgctatatgcaaaaatcggtgtttcttccggccgtggctcatgccgctccggttcgcccctcggcggtagaggagcagcaggctgaacagcctcttgaaccgctggaggatccggcggcacctcaatcggagctggatgaaatggcttggtgtttgttgcgatcaaagttgacggcgatgcgttctcattcaccttcttttggcgcccacctagccaaatgaggcttaatgataacgcgagaacgacacctccgacgatcaatttctgagaccccgaaagacgccggcgatgtttgtcggagaccagggatccagatgcatcaacctcatgtgccgcttgctgactatcgttattcatcccttcgcccccttcaggacgcgtttcacatcgggcctcaccgtgcccgtttgcggcctttggccaacgggatcgtaagcggtgttccagatacatagtactgtgtggccatccctcagacgccaacctcgggaaaccgaagaaatctcgacatcgctccctttaactgaatagttggcaacagcttccttgccatcaggattgatggtgtagatggagggtatgcgtacattgcccggaaagtggaataccgtcgtaaatccattgtcgaagacttcgagtggcaacagcgaacgatcgccttgggcgacgtagtgccaattactgtccgccgcaccaagggctgtgacaggctgatccaataaattctcagctttccgttgatattgtgcttccgcgtgtagtctgtccacaacagccttctgttgtgcctcccttcgccgagccgccgcatcgtcggcggggtaggcgaattggacgctgtaatagagatcgggctgctctttatcgaggtgggacagagtcttggaacttatactgaaaacataacggcgcatcccggagtcgcttgcggttagcacgattactggctgaggcgtgaggacctggcttgccttgaaaaatagataatttccccgcggtagggctgctagatctttgctatttgaaacggcaaccgctgtcaccgtttcgttcgtggcgaatgttacgaccaaagtagctccaaccgccgtcgagaggcgcaccacttgatcgggattgtaagccaaataacgcatgcgcggatctagcttgcccgccattggagtgtcttcagcctccgcaccagtcgcagcggcaaataaacatgctaaaatgaaaagtgcttttctgatcatggttcgctgtggcctacgtttgaaacggtatcttccgatgtctgataggaggtgacaaccagacctgccgggttggttagtctcaatctgccgggcaagctggtcaccttttcgtagcgaactgtcgcggtccacgtactcaccacaggcattttgccgtcaacgacgagggtccttttatagcgaatttgctgcgtgcttggagttacatcatttgaagcgatgtgctcgacctccaccctgccgcgtttgccaagaatgacttgaggcgaactgggattgggatagttgaagaattgctggtaatcctggcgcactgttggggcactgaagttcgataccaggtcgtaggcgtactgagcggtgtcggcatcataactctcgcgcaggcgaacgtactcccacaatgaggcgttaacgacggcctcctcttgagttgcaggcaatcgcgagacagacacctcgctgtcaacggtgccgtccggccgtatccatagatatacgggcacaagcctgctcaacggcaccattgtggctatagcgaacgcttgagcaacatttcccaaaatcgcgatagctgcgacagctgcaatgagtttggagagacgtcgcgccgatttcgctcgcgcggtttgaaaggcttctacttccttatagtgctcggcaaggctttcgcgcgccactagcatggcatattcaggccccgtcatagcgtccacccgaattgccgagctgaagatctgacggagtaggctgccatcgccccacattcagcgggaagatcgggcctttgcagctcgctaatgtgtcgtttgtctggcagccgctcaaagcgacaactaggcacagcaggcaatacttcatagaattctccattgaggcgaatttttgcgcgacctagcctcgctcaacctgagcgaagcgacggtacaagctgctggcagattgggttgcgccgctccagtaactgcctccaatgttgccggcgatcgccggcaaagcgacaatgagcgcatcccctgtcagaaaaaacatatcgagttcgtaaagaccaatgatcttggccgcggtcgtaccggcgaaggtgattacaccaagcataagggtgagcgcagtcgcttcggttaggatgacgatcgttgccacgaggtttaagaggagaagcaagagaccgtaggtgataagttgcccgatccacttagctgcgatgtcccgcgtgcgatcaaaaatatatccgacgaggatcagaggcccgatcgcgagaagcactttcgtgagaattccaacggcgtcgtaaactccgaaggcagaccagagcgtgccgtaaaggacccactgtgccccttggaaagcaaggatgtcctggtcgttcatcggaccgatttcggatgcgattttctgaaaaacggcctgggtcacggcgaacattgtatccaactgtgccggaacagtctgcagaggcaagccggttacactaaactgctgaacaaagtttgggaccgtcttttcgaagatggaaaccacatagtcttggtagttagcctgcccaacaattagagcaacaacgatggtgaccgtgatcacccgagtgataccgctacgggtatcgacttcgccgcgtatgactaaaataccctgaacaataatccaaagagtgacacaggcgatcaatggcgcactcaccgcctcctggatagtctcaagcatcgagtccaagcctgtcgtgaaggctacatcgaagatcgtatgaatggccgtaaacggcgccggaatcgtgaaattcatcgattggacctgaacttgactggtttgtcgcataatgttggataaaatgagctcgcattcggcgaggatgcgggcggatgaacaaatcgcccagccttaggggagggcaccaaagatgacagcggtcttttgatgctccttgcgttgagcggccgcctcttccgcctcgtgaaggccggcctgcgcggtagtcatcgttaataggcttgtcgcctgtacattttgaatcattgcgtcatggatctgcttgagaagcaaaccattggtcacggttgcctgcatgatattgcgagatcgggaaagctgagcagacgtatcagcattcgccgtcaagcgtttgtccatcgtttccagattgtcagccgcaatgccagcgctgtttgcggaaccggtgatctgcgatcgcaacaggtccgcttcagcatcactacccacgactgcacgatctgtatcgctggtgatcgcacgtgccgtggtcgacattggcattcgcggcgaaaacatttcattgtctaggtccttcgtcgaaggatactgatttttctggttgagcgaagtcagtagtccagtaacgccgtaggccgacgtcaacatcgtaaccatcgctatagtctgagtgagattctccgcagtcgcgagcgcagtcgcgagcgtctcagcctccgttgccgggtcgctaacaacaaactgcgcccgcgcgggctgaatatatagaaagctgcaggtcaaaactgttgcaataagttgcgtcgtcttcatcgtttcctaccttatcaatcttctgcctcgtggtgacgggccatgaattcgctgagccagccagatgagttgccttcttgtgcctcgcgtagtcgagttgcaaagcgcaccgtgttggcacgccccgaaagcacggcgacatattcacgcatatcccgcagatcaaattcgcagatgacgcttccactttctcgtttaagaagaaacttacggctgccgaccgtcatgtcttcacggatcgcctgaaattccttttcggtacatttcagtccatcgacataagccgatcgatctgcggttggtgatggatagaaaatcttcgtcatacattgcgcaaccaagctggctcctagcggcgattccagaacatgctctggttgctgcgttgccagtattagcatcccgttgttttttcgaacggtcaggaggaatttgtcgacgacagtcgaaaatttagggtttaacaaataggcgcgaaactcatcgcagctcatcacaaaacggcggccgtcgatcatggctccaatccgatgcaggagatatgctgcagcgggagcgcatacttcctcgtattcgagaagatgcgtcatgtcgaagccggtaatcgacggatctaactttacttcgtcaacttcgccgtcaaatgcccagccaagcgcatggccccggcaccagcgttggagccgcgctcctgcgccttcggcgggcccatgcaacaaaaattcacgtaaccccgcgattgaacgcatttgtggatcaaacgagagctgacgatggataccacggaccagacggcggttctcttccggagaaatcccaccccgaccatcactctcgatgagagccacgatccattcgcgcagaaaatcgtgtgaggctgctgtgttttctaggccacgcaacggcgccaacccgctgggtgtgcctctgtgaagtgccaaatatgttcctcctgtggcgcgaaccagcaattcgccaccccggtccttgtcaaagaacacgaccgtacctgcacggtcgaccatgctctgttcgagcatggctagaacaaacatcatgagcgtcgtcttacccctcccgataggcccgaatattgccgtcatgccaacatcgtgctcatgcgggatatagtcgaaaggcgttccgccattggtacgaaatcgggcaatcgcgttgccccagtggcctgagctggcgccctctggaaagttttcgaaagagacaaaccctgcgaaattgcgtgaagtgattgcgccagggcgtgtgcgccacttaaaattccccggcaattgggaccaataggccgcttccataccaataccttcttggacaaccacggcacctgcatccgccattcgtgtccgagcccgcgcgcccctgtccccaagactattgagatcgtctgcatagacgcaaaggctcaaatgatgtgagcccataacgaattcgttgctcgcaagtgcgtcctcagcctcggataatttgccgatttgagtcacggctttatcgccggaactcagcatctggctcgatttgaggctaagtttcgcgtgcgcttgcgggcgagtcaggaacgaaaaactctgcgtgagaacaagtggaaaatcgagggatagcagcgcgttgagcatgcccggccgtgtttttgcagggtattcgcgaaacgaatagatggatccaacgtaactgtcttttggcgttctgatctcgagtcctcgcttgccgcaaatgactctgtcggtataaatcgaagcgccgagtgagccgctgacgaccggaaccggtgtgaaccgaccagtcatgatcaaccgtagcgcttcgccaatttcggtgaagagcacaccctgcttctcgcggatgccaagacgatgcaggccatacgctttaagagagccagcgacaacatgccaaagatcttccatgttcctgatctggcccgtgagatcgttttccctttttccgcttagcttggtgaacctcctctttaccttccctaaagccgcctgtgggtagacaatcaacgtaaggaagtgttcattgcggaggagttggccggagagcacgcgctgttcaaaagcttcgttcaggctagcggcgaaaacactacggaagtgtcgcggcgccgatgatggcacgtcggcatgacgtacgaggtgagcatatattgacacatgatcatcagcgatattgcgcaacagcgtgttgaacgcacgacaacgcgcattgcgcatttcagtttcctcaagctcgaatgcaacgccatcaattctcgcaatggtcatgatcgatccgtcttcaagaaggacgatatggtcgctgaggtggccaatataagggagatagatctcaccggatctttcggtcgttccactcgcgccgagcatcacaccattcctctccctcgtgggggaaccctaattggatttgggctaacagtagcgcccccccaaactgcactatcaatgcttcttcccgcggtccgcaaaaatagcaggacgacgctcgccgcattgtagtctcgctccacgatgagccgggctgcaaaccataacggcacgagaacgacttcgtagagcgggttctgaacgataacgatgacaaagccggcgaacatcatgaataaccctgccaatgtcagtggcaccccaagaaacaatgcgggccgtgtggctgcgaggtaaagggtcgattcttccaaacgatcagccatcaactaccgccagtgagcgtttggccgaggaagctcgccccaaacatgataacaatgccgccgacgacgccggcaaccagcccaagcgaagcccgcccgaacatccaggagatcccgatagcgacaatgccgagaacagcgagtgactggccgaacggaccaaggataaacgtgcatatattgttaaccattgtggcggggtcagtgccgccacccgcagattgcgctgcggcgggtccggatgaggaaatgctccatgcaattgcaccgcacaagcttggggcgcagctcgatatcacgcgcatcatcgcattcgagagcgagaggcgatttagatgtaaacggtatctctcaaagcatcgcatcaatgcgcacctccttagtataagtcgaataagacttgattgtcgtctgcggatttgccgttgtcctggtgtggcggtggcggagcgattaaaccgccagcgccatcctcctgcgagcggcgctgatatgacccccaaacatcccacgtctcttcggattttagcgcctcgtgatcgtcttttggaggctcgattaacgcgggcaccagcgattgagcagctgtttcaacttttcgcacgtagccgtttgcaaaaccgccgatgaaattaccggtgttgtaagcggagatcgcccgacgaagcgcaaattgcttctcgtcaatcgtttcgccgcctgcataacgacttttcagcatgtttgcagcggcagataatgatgtgcacgcctggagcgcaccgtcaggtgtcagaccgagcatagaaaaatttcgagagtttatttgcatgaggccaacatccagcgaatgccgtgcatcgagacggtgcctgacgacttgggttgcttggctgtgatcttgccagtgaagcgtttcgccggtcgtgttgtcatgaatcgctaaaggatcaaagcgactctccaccttagctatcgccgcaagcgtagatgtcgcaactgatggggcacacttgcgagcaacatggtcaaactcagcagatgagagtggcgtggcaaggctcgacgaacagaaggagaccatcaaggcaagagaaagcgaccccgatctcttaagcataccttatctccttagctcgcaactaacaccgcctctcccgttggaagaagtgcgttgttttatgttgaagattatcgggagggtcggttactcgaaaattttcaattgcttctttatgatttcaattgaagcgagaaacctcgcccggcgtcttggaacgcaacatggaccgagaaccgcgcatccatgactaagcaaccggatcgacctattcaggccgcagttggtcaggtcaggctcagaacgaaaatgctcggcgaggttacgctgtctgtaaacccattcgatgaacgggaagcttccttccgattgctcttggcaggaatattggcccatgcctgcttgcgctttgcaaatgctcttatcgcgttggtatcatatgccttgtccgccagcagaaacgcactctaagcgattatttgtaaaaatgtttcggtcatgcggcggtcatgggcttgacccgctgtcagcgcaagacggatcggtcaaccgtcggcatcgacaacagcgtgaatcttggtggtcaaaccgccacgggaacgtcccatacagccatcgtcttgatcccgctgtttcccgtcgccgcatgttggtggacgcggacacaggaactgtcaatcatgacgacattctatcgaaagccttggaaatcacactcagaatatgatcccagacgtctgcctcacgccatcgtacaaagcgattgtagcaggttgtacaggaaccgtatcgatcaggaacgtctgcccagggcgggcccgtccggaagcgccacaagatgacattgatcacccgcgtcaacgcgcggcacgcgacgcggcttatttgggaacaaaggactgaacaacagtccattcgaaatcggtgacatcaaagcggggacgggttatcagtggcctccaagtcaagcctcaatgaatcaaaatcagaccgatttgcaaacctgatttatgagtgtgcggcctaaatgatgaaatcgtccttctagatcgcctccgtggtgtagcaacacctcgcagtatcgccgtgctgaccttggccagggaattgactggcaagggtgctttcacatgaccgctcttttggccgcgatagatgatttcgttgctgctttgggcacgtagaaggagagaagtcatatcggagaaattcctcctggcgcgagagcctgctctatcgcgacggcatcccactgtcgggaacagaccggatcattcacgaggcgaaagtcgtcaacacatgcgttataggcatcttcccttgaaggatgatcttgttgctgccaatctggaggtgcggcagccgcaggcagatgcgatctcagcgcaacttgcggcaaaacatctcactcacctgaaaaccactagcgagtctcgcgatcagacgaaggccttttacttaacgacacaatatccgatgtctgcatcacaggcgtcgctatcccagtcaatactaaagcggtgcaggaactaaagattactgatgacttaggcgtgccacgaggcctgagacgacgcgcgtagacagttttttgaaatcattatcaaagtgatggcctccgctgaagcctatcacctctgcgccggtctgtcggagagatgggcaagcattattacggtcttcgcgcccgtacatgcattggacgattgcagggtcaatggatctgagatcatccagaggattgccgcccttaccttccgtttcgagttggagccagcccctaaatgagacgacatagtcgacttgatgtgacaatgccaagagagagatttgcttaacccgatttttttgctcaagcgtaagcctattgaagcttgccggcatgacgtccgcgccgaaagaatatcctacaagtaaaacattctgcacaccgaaatgcttggtgtagacatcgattatgtgaccaagatccttagcagtttcgcttggggaccgctccgaccagaaataccgaagtgaactgacgccaatgacaggaatcccttccgtctgcagataggtaccatcgatagatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcggagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaattgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgac

SEQ ID NO: 8 - DNA, 13019 bp, artificial sequence
  vector used to construct PHP23236
gttacccggaccgaagcttagcccgggcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatttaaatacgcgttgatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatatcattgacacgcccggtcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattccggtccgggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcggaccgattaaactttaattcggtccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacagtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcg

SEQ ID NO: 9 - DNA, 15663 bp, Artificial Sequence
  PHP28647 destination vector for use with maize inbred-derived lines
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatctacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattccggtccgggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcggaccgaagctggccgctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcccccgggctgcaggtcgactctagaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacagtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttag

SEQ ID NO: 10 - DNA, 25 bp, Artificial Sequence
  attB1 site
acaagtttgtacaaaaaagcaggct

SEQ ID NO: 11 - DNA, 25 bp, Artificial Sequence
  attB2 site
accactttgtacaagaaagctgggt

SEQ ID NO: 12 - DNA, 55 bp, Artificial Sequence
  At2g04090 5′attB forward primer
ttaaacaagtttgtacaaaaaagcaggctcaacaatggaagatccacttttattg

SEQ ID NO: 13 - DNA, 50 bp, Artificial Sequence
  At2g04090 3′attB reverse primer
ttaaaccactttgtacaagaaagctgggttcagtatggggtaaaaaaaag

SEQ ID NO: 14 - DNA, 54 bp, Artificial Sequence
  VC062 primer
ttaaacaagtttgtacaaaaaagcaggctgcaattaaccctcactaaagggaac

SEQ ID NO: 15 - DNA, 53 bp, Artificial Sequence
  VC063 primer
ttaaaccactttgtacaagaaagctgggtgcgtaatacgactcactatagggc

SEQ ID NO: 16 - DNA, 50905 bp, Artificial Sequence
  destination vector for use with Gaspe-flint derived maize lines
ggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcggagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaattgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgactctagagctcgttcctcgaggcctcgaggcctcgaggaacggtacctgcggggaagcttacaataatgtgtgttgttaagtcttgttgcctgtcatcgtctgactgactttcgtcataaatcccggcctccgtaacccagctttgggcaagctcacggatttgatccggcggaacgggaatatcgagatgccgggctgaacgctgcagttccagctttccctttcgggacaggtactccagctgattgattatctgctgaagggtcttggttccacctcctggcacaatgcgaatgattacttgagcgcgatcgggcatccaattttctcccgtcaggtgcgtggtcaagtgctacaaggcacctttcagtaacgagcgaccgtcgatccgtcgccgggatacggacaaaatggagcgcagtagtccatcgagggcggcgaaagcctcgccaaaagcaatacgttcatctcgcacagcctccagatccgatcgagggtcttcggcgtaggcagatagaagcatggatacattgcttgagagtattccgatggactgaagtatggcttccatcttttctcgtgtgtctgcatctatttcgagaaagcccccgatgcggcgcaccgcaacgcgaattgccatactatccgaaagtcccagcaggcgcgcttgataggaaaaggtttcatactcggccgatcgcagacgggcactcacgaccttgaacccttcaactttcagggatcgatgctggttgatggtagtctcactcgacgtggctctggtgtgttttgacatagcttcctccaaagaaagcggaaggtctggatactccagcacgaaatgtgcccgggtagacggatggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaatatgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtactgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttgaagatggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtgaagagggacctatcggaacccctcaccaaatattgagtgtaggtttgaggccgctggccgcgtcctcagtcaccttttgagccagataattaagagccaaatgcaattggctcaggctgccatcgtccccccgtgcgaaacctgcacgtccgcgtcaaagaaataaccggcacctcttgctgtttttatcagttgagggcttgacggatccgcctcaagtttgcggcgcagccgcaaaatgagaacatctatactcctgtcgtaaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgcaagaaactctctcgtactaaacggcttagcgataaaatcacttgctcctagctcgagtgcaacaactttatccgtctcctcaaggcggtcgccactgataattatgattggaatatcagactttgccgccagatttcgaacgatctcaagcccatcttcacgacctaaatttagatcaacaaccacgacatcgaccgtcgcggaagagagtactctagtgaactgggtgctgtcggctaccgcggtcactttgaaggcgtggatcgtaaggtattcgataataagatgccgcatagcgacatcgtcatcgataagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacgaaaatgcgaaccaagtatttcaattttatgacaaaagttctcaatcgttgttacaagtgaaacgcttcgaggttacagctactattgattaaggagatcgcctatggtctcgccccggcgtcgtgcgtccgccgcgagccagatctcgcctacttcataaacgtcctcataggcacggaatggaatgatgacatcgatcgccgtagagagcatgtcaatcagtgtgcgatcttccaagctagcaccttgggcgctacttttgacaagggaaaacagtttcttgaatccttggattggattcgcgccgtgtattgttgaaatcgatcccggatgtcccgagacgacttcactcagataagcccatgctgcatcgtcgcgcatctcgccaagcaatatccggtccggccgcatacgcagacttgcttggagcaagtgctcggcgctcacagcacccagcccagcaccgttcttggagtagagtagtctaacatgattatcgtgtggaatgacgagttcgagcgtatcttctatggtgattagcctttcctggggggggatggcgctgatcaaggtcttgctcattgttgtcttgccgcttccggtagggccacatagcaacatcgtcagtcggctgacgacgcatgcgtgcagaaacgcttccaaatccccgttgtcaaaatgctgaaggatagcttcatcatcctgattttggcgtttccttcgtgtctgccactggttccacctcgaagcatcataacgggaggagacttctttaagaccagaaacacgcgagcttggccgtcgaatggtcaagctgacggtgcccgagggaacggtcggcggcagacagatttgtagtcgttcaccaccaggaagttcagtggcgcagagggggttacgtggtccgacatcctgctttctcagcgcgcccgctaaaatagcgatatcttcaagatcatcataagagacgggcaaaggcatcttggtaaaaatgccggcttggcgcacaaatgcctctccaggtcgattgatcgcaatttcttcagtcttcgggtcatcgagccattccaaaatcggcttcagaagaaagcgtagttgcggatccacttccatttacaatgtatcctatctctaagcggaaatttgaattcattaagagcggcggttcctcccccgcgtggcgccgccagtcaggcggagctggtaaacaccaaagaaatcgaggtcccgtgctacgaaaatggaaacggtgtcaccctgattcttcttcagggttggcggtatgttgatggttgccttaagggctgtctcagttgtctgctcaccgttattttgaaagctgttgaagctcatcccgccacccgagctgccggcgtaggtgctagctgcctggaaggcgccttgaacaacactcaagagcatagctccgctaaaacgctgccagaagtggctgtcgaccgagcccggcaatcctgagcgaccgagttcgtccgcgcttggcgatgttaacgagatcatcgcatggtcaggtgtctcggcgcgatcccacaacacaaaaacgcgcccatctccctgttgcaagccacgctgtatttcgccaacaacggtggtgccacgatcaagaagcacgatattgttcgttgttccacgaatatcctgaggcaagacacactttacatagcctgccaaatttgtgtcgattgcggtttgcaagatgcacggaattattgtcccttgcgttaccataaaatcggggtgcggcaagagcgtggcgctgctgggctgcagctcggtgggtttcatacgtatcgacaaatcgttctcgccggacacttcgccattcggcaaggagttgtcgtcacgcttgccttcttgtcttcggcccgtgtcgccctgaatggcgcgtttgctgaccccttgatcgccgctgctatatgcaaaaatcggtgtttcttccggccgtggctcatgccgctccggttcgcccctcggcggtagaggagcagcaggctgaacagcctcttgaaccgctggaggatccggcggcacctcaatcggagctggatgaaatggcttggtgtttgttgcgatcaaagttgacggcgatgcgttctcattcaccttcttttggcgcccacctagccaaatgaggcttaatgataacgcgagaacgacacctccgacgatcaatttctgagaccccgaaagacgccggcgatgtttgtcggagaccagggatccagatgcatcaacctcatgtgccgcttgctgactatcgttattcatcccttcgcccccttcaggacgcgtttcacatcgggcctcaccgtgcccgtttgcggcctttggccaacgggatcgtaagcggtgttccagatacatagtactgtgtggccatccctcagacgccaacctcgggaaaccgaagaaatctcgacatcgctccctttaactgaatagttggcaacagcttccttgccatcaggattgatggtgtagatggagggtatgcgtacattgcccggaaagtggaataccgtcgtaaatccattgtcgaagacttcgagtggcaacagcgaacgatcgccttgggcgacgtagtgccaattactgtccgccgcaccaagggctgtgacaggctgatccaataaattctcagctttccgttgatattgtgcttccgcgtgtagtctgtccacaacagccttctgttgtgcctcccttcgccgagccgccgcatcgtcggcggggtaggcgaattggacgctgtaatagagatcgggctgctctttatcgaggtgggacagagtcttggaacttatactgaaaacataacggcgcatcccggagtcgcttgcggttagcacgattactggctgaggcgtgaggacctggcttgccttgaaaaatagataatttccccgcggtagggctgctagatctttgctatttgaaacggcaaccgctgtcaccgtttcgttcgtggcgaatgttacgaccaaagtagctccaaccgccgtcgagaggcgcaccacttgatcgggattgtaagccaaataacgcatgcgcggatctagcttgcccgccattggagtgtcttcagcctccgcaccagtcgcagcggcaaataaacatgctaaaatgaaaagtgcttttctgatcatggttcgctgtggcctacgtttgaaacggtatcttccgatgtctgataggaggtgacaaccagacctgccgggttggttagtctcaatctgccgggcaagctggtcaccttttcgtagcgaactgtcgcggtccacgtactcaccacaggcattttgccgtcaacgacgagggtccttttatagcgaatttgctgcgtgcttggagttacatcatttgaagcgatgtgctcgacctccaccctgccgcgtttgccaagaatgacttgaggcgaactgggattgggatagttgaagaattgctggtaatcctggcgcactgttggggcactgaagttcgataccaggtcgtaggcgtactgagcggtgtcggcatcataactctcgcgcaggcgaacgtactcccacaatgaggcgttaacgacggcctcctcttgagttgcaggcaatcgcgagacagacacctcgctgtcaacggtgccgtccggccgtatccatagatatacgggcacaagcctgctcaacggcaccattgtggctatagcgaacgcttgagcaacatttcccaaaatcgcgatagctgcgacagctgcaatgagtttggagagacgtcgcgccgatttcgctcgcgcggtttgaaaggcttctacttccttatagtgctcggcaaggctttcgcgcgccactagcatggcatattcaggccccgtcatagcgtccacccgaattgccgagctgaagatctgacggagtaggctgccatcgccccacattcagcgggaagatcgggcctttgcagctcgctaatgtgtcgtttgtctggcagccgctcaaagcgacaactaggcacagcaggcaatacttcatagaattctccattgaggcgaatttttgcgcgacctagcctcgctcaacctgagcgaagcgacggtacaagctgctggcagattgggttgcgccgctccagtaactgcctccaatgttgccggcgatcgccggcaaagcgacaatgagcgcatcccctgtcagaaaaaacatatcgagttcgtaaagaccaatgatcttggccgcggtcgtaccggcgaaggtgattacaccaagcataagggtgagcgcagtcgcttcggttaggatgacgatcgttgccacgaggtttaagaggagaagcaagagaccgtaggtgataagttgcccgatccacttagctgcgatgtcccgcgtgcgatcaaaaatatatccgacgaggatcagaggcccgatcgcgagaagcactttcgtgagaattccaacggcgtcgtaaactccgaaggcagaccagagcgtgccgtaaaggacccactgtgccccttggaaagcaaggatgtcctggtcgttcatcggaccgatttcggatgcgattttctgaaaaacggcctgggtcacggcgaacattgtatccaactgtgccggaacagtctgcagaggcaagccggttacactaaactgctgaacaaagtttgggaccgtcttttcgaagatggaaaccacatagtcttggtagttagcctgcccaacaattagagcaacaacgatggtgaccgtgatcacccgagtgataccgctacgggtatcgacttcgccgcgtatgactaaaataccctgaacaataatccaaagagtgacacaggcgatcaatggcgcactcaccgcctcctggatagtctcaagcatcgagtccaagcctgtcgtgaaggctacatcgaagatcgtatgaatggccgtaaacggcgccggaatcgtgaaattcatcgattggacctgaacttgactggtttgtcgcataatgttggataaaatgagctcgcattcggcgaggatgcgggcggatgaacaaatcgcccagccttaggggagggcaccaaagatgacagcggtcttttgatgctccttgcgttgagcggccgcctcttccgcctcgtgaaggccggcctgcgcggtagtcatcgttaataggcttgtcgcctgtacattttgaatcattgcgtcatggatctgcttgagaagcaaaccattggtcacggttgcctgcatgatattgcgagatcgggaaagctgagcagacgtatcagcattcgccgtcaagcgtttgtccatcgtttccagattgtcagccgcaatgccagcgctgtttgcggaaccggtgatctgcgatcgcaacaggtccgcttcagcatcactacccacgactgcacgatctgtatcgctggtgatcgcacgtgccgtggtcgacattggcattcgcggcgaaaacatttcattgtctaggtccttcgtcgaaggatactgatttttctggttgagcgaagtcagtagtccagtaacgccgtaggccgacgtcaacatcgtaaccatcgctatagtctgagtgagattctccgcagtcgcgagcgcagtcgcgagcgtctcagcctccgttgccgggtcgctaacaacaaactgcgcccgcgcgggctgaatatatagaaagctgcaggtcaaaactgttgcaataagttgcgtcgtcttcatcgtttcctaccttatcaatcttctgcctcgtggtgacgggccatgaattcgctgagccagccagatgagttgccttcttgtgcctcgcgtagtcgagttgcaaagcgcaccgtgttggcacgccccgaaagcacggcgacatattcacgcatatcccgcagatcaaattcgcagatgacgcttccactttctcgtttaagaagaaacttacggctgccgaccgtcatgtcttcacggatcgcctgaaattccttttcggtacatttcagtccatcgacataagccgatcgatctgcggttggtgatggatagaaaatcttcgtcatacattgcgcaaccaagctggctcctagcggcgattccagaacatgctctggttgctgcgttgccagtattagcatcccgttgttttttcgaacggtcaggaggaatttgtcgacgacagtcgaaaatttagggtttaacaaataggcgcgaaactcatcgcagctcatcacaaaacggcggccgtcgatcatggctccaatccgatgcaggagatatgctgcagcgggagcgcatacttcctcgtattcgagaagatgcgtcatgtcgaagccggtaatcgacggatctaactttacttcgtcaacttcgccgtcaaatgcccagccaagcgcatggccccggcaccagcgttggagccgcgctcctgcgccttcggcgggcccatgcaacaaaaattcacgtaaccccgcgattgaacgcatttgtggatcaaacgagagctgacgatggataccacggaccagacggcggttctcttccggagaaatcccaccccgaccatcactctcgatgagagccacgatccattcgcgcagaaaatcgtgtgaggctgctgtgttttctaggccacgcaacggcgccaacccgctgggtgtgcctctgtgaagtgccaaatatgttcctcctgtggcgcgaaccagcaattcgccaccccggtccttgtcaaagaacacgaccgtacctgcacggtcgaccatgctctgttcgagcatggctagaacaaacatcatgagcgtcgtcttacccctcccgataggcccgaatattgccgtcatgccaacatcgtgctcatgcgggatatagtcgaaaggcgttccgccattggtacgaaatcgggcaatcgcgttgccccagtggcctgagctggcgccctctggaaagttttcgaaagagacaaaccctgcgaaattgcgtgaagtgattgcgccagggcgtgtgcgccacttaaaattccccggcaattgggaccaataggccgcttccataccaataccttcttggacaaccacggcacctgcatccgccattcgtgtccgagcccgcgcgcccctgtccccaagactattgagatcgtctgcatagacgcaaaggctcaaatgatgtgagcccataacgaattcgttgctcgcaagtgcgtcctcagcctcggataatttgccgatttgagtcacggctttatcgccggaactcagcatctggctcgatttgaggctaagtttcgcgtgcgcttgcgggcgagtcaggaacgaaaaactctgcgtgagaacaagtggaaaatcgagggatagcagcgcgttgagcatgcccggccgtgtttttgcagggtattcgcgaaacgaatagatggatccaacgtaactgtcttttggcgttctgatctcgagtcctcgcttgccgcaaatgactctgtcggtataaatcgaagcgccgagtgagccgctgacgaccggaaccggtgtgaaccgaccagtcatgatcaaccgtagcgcttcgccaatttcggtgaagagcacaccctgcttctcgcggatgccaagacgatgcaggccatacgctttaagagagccagcgacaacatgccaaagatcttccatgttcctgatctggcccgtgagatcgttttccctttttccgcttagcttggtgaacctcctctttaccttccctaaagccgcctgtgggtagacaatcaacgtaaggaagtgttcattgcggaggagttggccggagagcacgcgctgttcaaaagcttcgttcaggctagcggcgaaaacactacggaagtgtcgcggcgccgatgatggcacgtcggcatgacgtacgaggtgagcatatattgacacatgatcatcagcgatattgcgcaacagcgtgttgaacgcacgacaacgcgcattgcgcatttcagtttcctcaagctcgaatgcaacgccatcaattctcgcaatggtcatgatcgatccgtcttcaagaaggacgatatggtcgctgaggtggccaatataagggagatagatctcaccggatctttcggtcgttccactcgcgccgagcatcacaccattcctctccctcgtgggggaaccctaattggatttgggctaacagtagcgcccccccaaactgcactatcaatgcttcttcccgcggtccgcaaaaatagcaggacgacgctcgccgcattgtagtctcgctccacgatgagccgggctgcaaaccataacggcacgagaacgacttcgtagagcgggttctgaacgataacgatgacaaagccggcgaacatcatgaataaccctgccaatgtcagtggcaccccaagaaacaatgcgggccgtgtggctgcgaggtaaagggtcgattcttccaaacgatcagccatcaactaccgccagtgagcgtttggccgaggaagctcgccccaaacatgataacaatgccgccgacgacgccggcaaccagcccaagcgaagcccgcccgaacatccaggagatcccgatagcgacaatgccgagaacagcgagtgactggccgaacggaccaaggataaacgtgcatatattgttaaccattgtggcggggtcagtgccgccacccgcagattgcgctgcggcgggtccggatgaggaaatgctccatgcaattgcaccgcacaagcttggggcgcagctcgatatcacgcgcatcatcgcattcgagagcgagaggcgatttagatgtaaacggtatctctcaaagcatcgcatcaatgcgcacctccttagtataagtcgaataagacttgattgtcgtctgcggatttgccgttgtcctggtgtggcggtggcggagcgattaaaccgccagcgccatcctcctgcgagcggcgctgatatgacccccaaacatcccacgtctcttcggattttagcgcctcgtgatcgtcttttggaggctcgattaacgcgggcaccagcgattgagcagctgtttcaacttttcgcacgtagccgtttgcaaaaccgccgatgaaattaccggtgttgtaagcggagatcgcccgacgaagcgcaaattgcttctcgtcaatcgtttcgccgcctgcataacgacttttcagcatgtttgcagcggcagataatgatgtgcacgcctggagcgcaccgtcaggtgtcagaccgagcatagaaaaatttcgagagtttatttgcatgaggccaacatccagcgaatgccgtgcatcgagacggtgcctgacgacttgggttgcttggctgtgatcttgccagtgaagcgtttcgccggtcgtgttgtcatgaatcgctaaaggatcaaagcgactctccaccttagctatcgccgcaagcgtagatgtcgcaactgatggggcacacttgcgagcaacatggtcaaactcagcagatgagagtggcgtggcaaggctcgacgaacagaaggagaccatcaaggcaagagaaagcgaccccgatctcttaagcataccttatctccttagctcgcaactaacaccgcctctcccgttggaagaagtgcgttgttttatgttgaagattatcgggagggtcggttactcgaaaattttcaattgcttctttatgatttcaattgaagcgagaaacctcgcccggcgtcttggaacgcaacatggaccgagaaccgcgcatccatgactaagcaaccggatcgacctattcaggccgcagttggtcaggtcaggctcagaacgaaaatgctcggcgaggttacgctgtctgtaaacccattcgatgaacgggaagcttccttccgattgctcttggcaggaatattggcccatgcctgcttgcgctttgcaaatgctcttatcgcgttggtatcatatgccttgtccgccagcagaaacgcactctaagcgattatttgtaaaaatgtttcggtcatgcggcggtcatgggcttgacccgctgtcagcgcaagacggatcggtcaaccgtcggcatcgacaacagcgtgaatcttggtggtcaaaccgccacgggaacgtcccatacagccatcgtcttgatcccgctgtttcccgtcgccgcatgttggtggacgcggacacaggaactgtcaatcatgacgacattctatcgaaagccttggaaatcacactcagaatatgatcccagacgtctgcctcacgccatcgtacaaagcgattgtagcaggttgtacaggaaccgtatcgatcaggaacgtctgcccagggcgggcccgtccggaagcgccacaagatgacattgatcacccgcgtcaacgcgcggcacgcgacgcggcttatttgggaacaaaggactgaacaacagtccattcgaaatcggtgacatcaaagcggggacgggttatcagtggcctccaagtcaagcctcaatgaatcaaaatcagaccgatttgcaaacctgatttatgagtgtgcggcctaaatgatgaaatcgtccttctagatcgcctccgtggtgtagcaacacctcgcagtatcgccgtgctgaccttggccagggaattgactggcaagggtgctttcacatgaccgctcttttggccgcgatagatgatttcgttgctgctttgggcacgtagaaggagagaagtcatatcggagaaattcctcctggcgcgagagcctgctctatcgcgacggcatcccactgtcgggaacagaccggatcattcacgaggcgaaagtcgtcaacacatgcgttataggcatcttcccttgaaggatgatcttgttgctgccaatctggaggtgcggcagccgcaggcagatgcgatctcagcgcaacttgcggcaaaacatctcactcacctgaaaaccactagcgagtctcgcgatcagacgaaggccttttacttaacgacacaatatccgatgtctgcatcacaggcgtcgctatcccagtcaatactaaagcggtgcaggaactaaagattactgatgacttaggcgtgccacgaggcctgagacgacgcgcgtagacagttttttgaaatcattatcaaagtgatggcctccgctgaagcctatcacctctgcgccggtctgtcggagagatgggcaagcattattacggtcttcgcgcccgtacatgcattggacgattgcagggtcaatggatctgagatcatccagaggattgccgcccttaccttccgtttcgagttggagccagcccctaaatgagacgacatagtcgacttgatgtgacaatgccaagagagagatttgcttaacccgatttttttgctcaagcgtaagcctattgaagcttgccggcatgacgtccgcgccgaaagaatatcctacaagtaaaacattctgcacaccgaaatgcttggtgtagacatcgattatgtgaccaagatccttagcagtttcgcttggggaccgctccgaccagaaataccgaagtgaactgacgccaatgacaggaatcccttccgtctgcagataggtaccatcgatagatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttacccggaccgaagcttgaagttcctattccgaagttcctattctctagaaagtataggaacttcagatctcgatgctcaccctgttgtttggtgttacttctgcaggtcgactctagaggatccaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggactggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatccgtcgaccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgcgatctggggaattcccatggacaccggtgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtaccggtctctacgtacagtccggactggcgccttggcgcgccgatcatccacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatttaaatacgcgttgatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatatcattgacacgcccggtcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattccggtccgggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcggaccgattaaactttaattcggtccgaagcttgaagttcctattccgaagttcctattctccagaaagtataggaacttcgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgatagaggtgaaaccgattaacgcagaggatacctatgaactaaggcatagaatactcagaccaaaccagccgatagaagcgtgtatgtttgaaagcgatttacttcgtggtgcatttcacttaggcggctattacgggggcaaactgatttccatagcttcattccaccaggccgagcactcagaactccaaggccagaaacagtaccagctccgaggtatggctaccttggaaggttatcgtgagcagaaggcgggatcgagtctaattaaacacgctgaagaaattcttcgtaagaggggggcggacttgctttggtgtaatgcgcggacatccgcctcaggctactacaaaaagttaggcttcagcgagcagggagaggtattcgacacgccgccagtaggacctcacatcctgatgtataaaaggatcacataactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcagagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacagtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgca

SEQ ID NO: 17 - DNA, 1457 bp, Arabidopsis thaliana
caacaatggaagatccacttttattgggagatgatcagttaatcactagaaacctcaagtcaacgccgacatggtggatgaattttacggcggagctgaagaacgtcagctctatggcggcgcctatggccaccgtgacagtgtctcaatatctattgcccgtgatctcggtcatggtcgccggccactgcggtgaactccagctgtctggtgtcactcttgccactgctttcgcaaacgtctccggcttcggcatcatgtatggtttagtgggtgcacttgaaactctatgtggccaagcttatggagcaaaacaatacactaaaatcggaacttacactttctctgcaatagtctcaaacgtacctatagttgttctcatatcgattctctggttttacatggacaaactctttgtttcacttggacaagatcctgacatctccaaggtagctggttcttacgcggtttgtcttataccggcattgttagctcaagcagtgcaacaacctttgactcggtttctccagactcagggtttggttcttcctcttctctactgtgccataaccacccttttattccatataccagtttgtttgattctggtttacgcgtttggtcttggaagcaatggagccgccttggctattggtttgtcttactggtttaatgtcttgattcttgctttatatgtgagattttcaagcgcttgcgagaagactcgcggctttgtatccgatgatttcgtgttgagtgtcaagcagttttttcagtatgggataccatcagcagcaatgacaaccatagaatggtcgttgtttgagctccttatcttatcttcaggactcctcccaaacccgaaactcgagacctctgttctttccatttgtcttacaacatcatctctccactgtgtcattccaatgggtatcggggctgctggaagcacacggatttcaaacgaattgggagcgggaaatccggaggttgctaggctggcagtgtttgccggtattttcctttggttcctagaggctaccatttgtagcacacttctgttcacttgcaaaaatatttttggctacgcgttcagcaatagcaaagaagttgtggactatgtcacggagctatcttcgctgctttgtctttcatttatggtcgatggattttcttcagtgcttgatggggttgctaggggaagtgggtggcaaaatattggagcttgggcaaatgtggtggcttactatctcctaggagctcctgttggatttttcttaggattttggggtcatatgaacggcaaagggctatggattggtgtgatcgttgggtccactgctcaagggatcatactagctatagtcactgcttgcctgagttgggaggagcaggtcaatagcaatcttaaatatatttttggacatttgatgaatcttttttttaccccatactga

SEQ ID NO: 18 - protein, 483 aa, Arabidopsis thaliana
MEDPLLLGDDQLITRNLKSTPTWWMNFTAELKNVSSMAAPMATVTVSQYLLPVISVMVAGHCGELQLSGVTLATAFANVSGFGIMYGLVGALETLCGQAYGAKQYTKIGTYTFSAIVSNVPIVVLISILWFYMDKLFVSLGQDPDISKVAGSYAVCLIPALLAQAVQQPLTRFLQTQGLVLPLLYCAITTLLFHIPVCLILVYAFGLGSNGAALAIGLSYWFNVLILALYVRFSSACEKTRGFVSDDFVLSVKQFFQYGIPSAAMTTIEWSLFELLILSSGLLPNPKLETSVLSICLTTSSLHCVIPMGIGAAGSTRISNELGAGNPEVARLAVFAGIFLWFLEATICSTLLFTCKNIFGYAFSNSKEVVDYVTELSSLLCLSFMVDGFSSVLDGVARGSGWQNIGAWANVVAYYLLGAPVGFFLGFWGHMNGKGLWIGVIVGSTAQGIILAIVTACLSWEEQVNSNLKYIFGHLMNLFFTPY

SEQ ID NO: 19 - DNA, 1896 bp, zea mays
gtgcgcacaggcgacaggcccatccacacatgcagctccaagcatcggtacggcactgagcatcggtgaggcaggcgacgcgcgccgcgcgagagaggcgcctcgcctggggagacagggagggtggccgagcccgagatggccgccgcgagggaggaggacgaggcggcacggccgctgcttctgctgccgcggacggcgcaggaggaccagaagtggtggaggcggtgggcgcgcgaggccgggtgggtggggtacctggccctgcccatggtggtggtgaacctgtcgcagtacgccgtccaggtgtcgtccaacatgatggtggggcacctccccggcgtgctcccgctctcctccgccgccatcgccacctccctcgccaacgtctccgggttcagcctcctgatcggaatggcaagtgcactggagacgctatgcggccaggcctacggtgcgaagcagtaccataagctaggcctagacacctacagagcggtcgtcaccctcctggtggtctgcgtccccctctcgctcctctgggtgttcatggacaagatcctggtcctcataggccaggaccctctcatctcgcaaggcgccgggaggtacatggtctggctgatcccggggctcttcgccaacgcggtgatccagccgctcaccaagttcctgcagacgcagagcctcatctacccgctgctgctgtcgtccgcggcgacggcggcggtccacgtccccctgtgctacgtgatggtgttcaagaccgggctcgggtacacgggcgccgccctgaccataagcatatcgtactggctgaacgtggccatgctcgtcggatacatcgccttctccagctcttgcaaggagacgcgcgcgcgcccgacggtcgaggtcttcaggggagtcgacgcgttcctgcgtctcgccctgccttctgcgctcatgatgtgtcttgaatggtggtcgtttgagctccttactctcatgtcagggcttctacccaacccagagctgcagacctcagtgctttcgatctgtctcacgagtgtaacattactcttcactataccttttggccttggagctgctggaagcacgcgagtggcaaatgagctgggcgctgggaaccctgacggagctcgatcagcagtccgcgtggtgctgtccatggcggggatcgacgcggtcgtcgtgagcggaagccttctggcggcccggcgcctcgtgggcatcgcgtacagcagcgaggaggaggtcatatctgcggtcgccgccatggttcctctggtctgcatcaccgccataactgactgcctacaaggaatcctctcaggcgtcgcccgcgggtgcgggtggcagcacctgggcgcgtacgtgaacctcggctcgttctacctgctggggatcccgatggcgatcctcctcggctttgtgctgcgcatggggtcgagggggctctggatgggcatcgtctgcggctccctgtcgcagaccacgctcatgtccgccatcacgttcttcaccgactggaacaagatggctgagaaggctagagagagggtgttcagcgacaagcagccacaagagccggggccatgatttgtacgccgactagcggcagctgcacatttttccagaccggttttgcaggggatcggaaggcgtttgcttgtcattccgtcgcattcacgaggagggagggggtcatgcctccctcctgcaaagtttctcacaaggcaaatgggttcttgctttgaacttcgccttcctacgtttgatttttgtatcatgtaaagtggagaggaacatggaacgaacaaattggacctcatatatgactaacaaggcgaaaatcatctcactaaaaaaaaaaaaaaaaaaaaaaaaaaaaaa

SEQ ID NO: 20 - protein, 487 aa, zea mays
MAAAREEDEAARPLLLLPRTAQEDQKWWRRWAREAGWVGYLALPMVVVNLSQYAVQVSSNMMVGHLPGVLPLSSAAIATSLANVSGFSLLIGMASALETLCGQAYGAKQYHKLGLDTYRAVVTLLVVCVPLSLLWVFMDKILVLIGQDPLISQGAGRYMVWLIPGLFANAVIQPLTKFLQTQSLIYPLLLSSAATAAVHVPLCYVMVFKTGLGYTGAALTISISYWLNVAMLVGYIAFSSSCKETRARPTVEVFRGVDAFLRLALPSALMMCLEWWSFELLTLMSGLLPNPELQTSVLSICLTSVTLLFTIPFGLGAAGSTRVANELGAGNPDGARSAVRVVLSMAGIDAVVVSGSLLAARRLVGIAYSSEEEVISAVAAMVPLVCITAITDCLQGILSGVARGCGWQHLGAYVNLGSFYLLGIPMAILLGFVLRMGSRGLWMGIVCGSLSQTTLMSAITFFTDWNKMAEKARERVFSDKQPQEPGP

SEQ ID NO: 21 - DNA, 2002 bp, zea mays
aagactccacctaaaacggccaggccagacttggtcgtgccggcgtgcggcgtgcccccgagctagctgtggacctgggagccacgacggcgatcatgggctcttcttccggggcgccgctgctcgtcgctcacccgagccgggggaaggaggacctgggagatcagcgccgcctccgctggtgctgcggcgtttcgtcggaagggcggtgggcggaggcgacggcggaggccgggcggctggcggcgctggcggcgcccatgatcgccgtggcgctgctgcagctcatgatgcaggtcatctccaccatcatggtgggccacctcggcgaggtgcccctcgccggcgccgctatcgccggctcgctcaccaacgtctccggatttagcgtcctcatgggactggcatgcggattggaaactatttgtggacaggccttcggggcagaacagtatcataaggtagctttatatacctaccggtctatagtcgtactccttattgcgagtgtacccatggccattttatgggttttcctcccagacgtacttcctctcataggtcaggatccacaaatagcgattgaggccgggaggtatgccttgtggcttatccctggtttattcgccttcagtgtggctcaatgcctttcaaagttcctccaatctcagagcttgatttttcccctggttctgagctccttgactacactcgctgtcttcattcctttgtgctggttcatggtttacaaagttgggatgggtaatgctggagctgcttttgcagtcagcatctgtgactgggttgaagtcacggttcttggtctttatattaagttctcaccttcttgtgagaaaactcgtgctccattcacgtgggaagcttttcaagggattggcagtttcatgcggttggctgtaccttcggctcttatggtttgtcttgaatggtggtcatatgagctgcttgttctgctttctgggatgttaccaaatgcagcacttgaaacttctgtgctctctatatgtatctctacagtaatactggtgtacaatctcccatatggcatcggaacagctgcaagcgtgcgtgtgtcaaatgaactaggcgcggggaacccagatagtgcccgcttagtagtagttgtcgccttatccattataatcttcacggcagttctggtgagcgtaactcttctatcgttgcgccatttcatcggaattgctttcagcaatgaggaggaggttgtaaattatgtcaccagaatggtaccgttgctttcgatttcagttattactgacaacctccaaggagtcctttcaggtatttctagaggctgtggttggcagcatttaggcgcctatgttaacttgggcgcgttctatcttgttggcattcctgtggcgctcgttgccggttttgctttgcatctaggaggagctgggttctggattggtatgatagctggtggagccacacaggtcactctcctaacaatcattactgcaatgacaaactggcggaagatggctgacaaagctagagatagagtatatgagggaagtctgcctatacaagcaaattgattattacgaacttgaagtcatatccgattatctccaaatttcgctgcatcctagttgtttgttgaaagagctatccatgccacggacaccctaccccccacccaaaaaaaagttggcaatgaaagtcgtaaaagatgggctatttttatacctactttgaactaggtgatgatgagaaccgtcttatcttataggtgatgatgctgtgtctagcttatagcttagactatctctaggctcaagcaatttatttatcttcatctcattttaaactctattttgtaaacatataggacctgtttggttcagtcccagtagacctgtctggcctaggcagcgtcccaggccattgattttggacgcttgaaaaaaaaaaaaaaaaaaaaaaaaaaaaaaaaaaaaaaaaaaaaaaaaaa

SEQ ID NO: 22 - protein, 496 aa, Zea mays
MGSSSGAPLLVAHPSRGKEDLGDQRRLRWCCGVSSEGRWAEATAEAGRLAALAAPMIAVALLQLMMQVISTIMVGHLGEVPLAGAAIAGSLTNVSGFSVLMGLACGLETICGQAFGAEQYHKVALYTYRSIVVLLIASVPMAILWVFLPDVLPLIGQDPQIAIEAGRYALWLIPGLFAFSVAQCLSKFLQSQSLIFPLVLSSLTTLAVFIPLCWFMVYKVGMGNAGAAFAVSICDWVEVTVLGLYIKFSPSCEKTRAPFTWEAFQGIGSFMRLAVPSALMVCLEWWSYELLVLLSGMLPNAALETSVLSICISTVILVYNLPYGIGTAASVRVSNELGAGNPDSARLVVVVALSIIIFTAVLVSVTLLSLRHFIGIAFSNEEEVVNYVTRMVPLLSISVITDNLQGVLSGISRGCGWQHLGAYVNLGAFYLVGIPVALVAGFALHLGGAGFWIGMIAGGATQVTLLTIITAMTNWRKMADKARDRVYEGSLPIQAN

SEQ ID NO: 23 - DNA, 1914 bp, zea mays
ggcacggccgagccgccttcgtcttggccgcagcggtgcgcgctctccatggcgtcctaattagcctagccgcaacgccgcgcccgtgcctgcggcatggatgaaccccttcttggcaatggtcttaagaccagcgggaaaagagagagcctggtagtggccgaggtcagaaaacagatgtaccttgctgggccactcatcgctgcatggatcctacagaacattgtccagatgatatctatcatgtttgtcggtcaccttggtgagcttgccctctccagtgcctccatcgccacctcctttgcaggcgttactggcttcagcttattgtctggcatggcgagcagcttggacacactgtgtgggcaatcgttcggggcaaagcagtactatcttcttggcatctacaagcagagggcaatccttgtgctcactctagtcagccttgtggttgcgattatctggtcatacactggacagatccttctactctttggtcaggatccagagattgcagctggggcagggagctatatccggtggatgattcctgcgctatttgtgtatggtccactacagtgccatgtccggtttctgcaaacgcagaacatagtcctcccagtgatgctgagctcaggtgccacagcactgaaccatctgctggtgtgctggctgctggtgtacaagattggtatgggcaacaagggtgctgccttggccaatgccatctcatactttaccaatgtatcaatcctggcaatttacgtcaggcttgcaccagcctgtagaaacacctggagagggttctcaaaggaggcttttcatgacataaccagcttcttgaggcttggtattccatctgcgctgatggtttgcttggagtggtggtcatttgagctcctggtacttctttctggacttctccccaatccaaagcttgagacatcggttttgtccatttccttaaacacaggctctttagcgtttatgatcccttttgggcttagcgcagccataagcacccgtgtttcaaatgaacttggtgctgggcgaccccatgctgcccatctggctacccgtgtggtcatggtgctggccatcgtggttggcgtattaatcggactagctatgattttggtgcgcaatatctgggggtatgcatacagtaatgagaaggaggtggtcaaatacatctccaaaatgatgccgatcctagccgtgtcattcttgttcgattgcgtgcagtgtgttctttcaggtgttgctaggggctgtggatggcaaaagattggggcttgtgttaatcttggtgcatactaccttataggaattccagctgccttctgtttcgcctttatgtaccatctaggtggaatgggactttggttgggaataatctgcgcactcgtcatacagatgctattgcttctcacaattaccttgtgcagcaactgggagaaagaagctttgaaggcaaaggacagagtatttagtacatccgtaccagctgacatgatgacatgatatttctgaagatattttttgaaggaaatgcccatgaaactttgccaaggaacatactaagaattggttgttcagattcaaggcctttctccatgccatcagaatgatgcttgggtctagtaatagctcctgagcagtctgttttttacttttggtcaatactcaatacatggaagatgtttggtttgaagaaccacgccatcctagatgaggtggtatattataagttgatataacggctccatactagttattgaggaatgagacgatgattatctcatgtaataataatgcttttccccctctagttattgatgtaagacaattgaggcaaatgaacaaaatgtcacatttgattaaataaaaaaaaaaaaaaaaaaaaaaaaaaaa

SEQ ID NO: 24 - protein, 475 aa, zea mays
MDEPLLGNGLKTSGKRESLVVAEVRKQMYLAGPLIAAWILQNIVQMISIMFVGHLGELALSSASIATSFAGVTGFSLLSGMASSLDTLCGQSFGAKQYYLLGIYKQRAILVLTLVSLVVAIIWSYTGQILLLFGQDPEIAAGAGSYIRWMIPALFVYGPLQCHVRFLQTQNIVLPVMLSSGATALNHLLVCWLLVYKIGMGNKGAALANAISYFTNVSILAIYVRLAPACRNTWRGFSKEAFHDITSFLRLGIPSALMVCLEWWSFELLVLLSGLLPNPKLETSVLSISLNTGSLAFMIPFGLSAAISTRVSNELGAGRPHAAHLATRVVMVLAIVVGVLIGLAMILVRNIWGYAYSNEKEVVKYISKMMPILAVSFLFDCVQCVLSGVARGCGWQKIGACVNLGAYYLIGIPAAFCFAFMYHLGGMGLWLGIICALVIQMLLLLTITLCSNWEKEALKAKDRVFSTSVPADMMT

SEQ ID NO: 25 - DNA, 1778 bp, zea mays
gacggttccgttcagtgtctggcggcgcatagatagatagccatggcgaagaagccagtggaggaagcgctcctcgcagcggcagacgagcaggagagcctgagcgtgcgcgaggagctgaagaagcagctatggctggctgggcccatgatcgccggcgcgctcctgcagaacgtgatccagatgatctccgtcatgtacgtgggacaccttggagagctgcccctcgctggcgcatccatggccaactccttcgccaccgtcaccggcctcagcctgctgctaggcatggcaagcgctctagacacactgtgcggccaagcgttcggcgcgaggcagtactatctcctgggcatctacaagcagcgcgccatgttcctcctgaccctggtgagcgtccctctgtcagtggtgtggttctacaccggcgagatcctcctcctgttcggccaggacccggacatcgccgcagaggccggcacctacgcccggtggatgatcccgctgctgttcgcgtacggcctgctgcagtgccacgtccggttcctccagacgcagaacatcgtggtgcccgtgatggcgagcgccggcgccgccgcggcctgccacgtcgtcgtctgctgggcgctggtgtacgcgctcgggatgggcagcaagggcgcggcgctcagcaactccatctcctactgggtcaacgtggccgtgctggccgtgtacgtgagggtgtccagcgcctgcaaggagacgtggacgggcttctccacggaggccttccgcgacgcgctcggcttcttcaggcttgcggtcccgtccgctctcatggtctgcttggagatgtggtcgtttgaactcattgtgctcctctcgggccttcttcccaacccaaaactggagacctccgtgctgtcgatcagccttaacactgctgcgttcgtgtggatgatcccctttgggcttagctcagctatcagcactcgcgtgtcgaatgagctgggtgccgggcgtcctcgagccgcccgccttgcggtgcgtgtcgttgtgttgctgtctgtcgccgaagggctaggcgtgggcctcatcctggtgtgcgtgcgctacgtctggggccatgcctacagcaacgtcgaggaggtggtgacgtacgtggccaacatgatgctggtcattgcagtgtccaacttcttcgatggtatccagtgcgtgctttcaggtgtggctagaggctgtggatggcagaagatcggtgcctgcatcaaccttggcgcctactacatcgtcggcatcccctctgcttacctcatagcgttcgtcctgcgtgtcggtgggacgggactctggttgggcatcatatgtgggctcatcgtacagctccttctgctcgcgatcatcactctctgcaccaactgggatagcgaggcaacgaaggcaaagaacagagtgttcaattcttcctctccagcatccggaacgtgaacggattggcggtgaagtaaacggcgagcaaacacggaaactataggagttatgcctgtagttttggaaacttcagcacctctgatcctctccgattttctggctcccacattaactctaatggttagcattgacacttgcagtctggattttgaaacataaatgcgttccttgaacagagtaggaagggaccaatggaaaacttatacaatagtgttagatcgcatgcaacattgtcctccaaagtgacctcatcacacaacaaaacgtgggtaattccaaaaaaaaaaaaaaaaaaaaaaaaa

SEQ ID NO: 26 - protein, 476 aa, zea mays
MAKKPVEEALLAAADEQESLSVREELKKQLWLAGPMIAGALLQNVIQMISVMYVGHLGELPLAGASMANSFATVTGLSLLLGMASALDTLCGQAFGARQYYLLGIYKQRAMFLLTLVSVPLSVVWFYTGEILLLFGQDPDIAAEAGTYARWMIPLLFAYGLLQCHVRFLQTQNIVVPVMASAGAAAACHVVVCWALVYALGMGSKGAALSNSISYWVNVAVLAVYVRVSSACKETWTGFSTEAFRDALGFFRLAVPSALMVCLEMWSFELIVLLSGLLPNPKLETSVLSISLNTAAFVWMIPFGLSSAISTRVSNELGAGRPRAARLAVRVVVLLSVAEGLGVGLILVCVRYVWGHAYSNVEEVVTYVANMMLVIAVSNFFDGIQCVLSGVARGCGWQKIGACINLGAYYIVGIPSAYLIAFVLRVGGTGLWLGIICGLIVQLLLLAIITLCTNWDSEATKAKNRVFNSSSPASGT

SEQ ID NO: 27 - protein, 483 aa, Arabidopsis thaliana; identical to SEQ ID NO: 18

SEQ ID NO: 28 - protein, 487 aa, zea mays; identical to SEQ ID NO: 20

SEQ ID NO: 29 - protein, 487 aa, zea mays; identical to SEQ ID NO: 20

SEQ ID NO: 30 - protein, 505 aa, sorghum bicolor
MGSSEAPLLLAHPGEGKEDPGADVGDRRRLRCCWWWRRCGGASSEGWWAEVTAEAGRLAALAAPMIAVALLQLMMQLISTIMVGHLGEVPLAGAAIANSLTNVSGFSVLMGLASGLETICGQAFGAEQYHKVALYTYRSIIVLLIASVPMAITWVFIPDVLPLIGQDPQIASEAGRYALWLIPGLFAFSVAQCLSKFLQSQSLIFPMVLSSFTTLAVFIPLCWFMVYKVGMGNAGAAFAVSICDWVEVTVLGLYIKFSPSCEKTRAPFTWEAFRGIGNFMRLAVPSALMICLEWWSYELLVLLCGVLPNAALETSVLSICISTVVLVYNLPYGIGTAASVRVSNELGAGNPDGARLVVVVALSIIICTAVLLSITLLSFRHFVGIAFSNEEEVVNHVTRMVPLLSISVLTDNLQGVLSGISRGCGWQHLGAYVNLGAFYLIGIPVGLVAGFALHLGGAGFWIGMIAGGATQVTLLSVITAMTNWQKMADKARDRVYEGSLPTQAD

SEQ ID NO: 31 - protein, 496 aa, zea mays
MGSSSGAPLLVAHPSRGKEDLGDQRRLRWCCGVSSEGRWAEATAEAGRLAALAAPMIAVALLQLMMQVISTIMVGHLGEVPLAGAAIAGSLTNVSGFSVLMGLACGLETICGQAFGAEQYHKVALYTYRSIVVLLIASVPMAILWVFLPDVLPLICQDPQIAIEAGRYALWLIPGLFAFSVAQCLSKFLQSQSLIFPLVLSSLTTLAVFIPLCWFMVYKVGMGNAGAAFAVSICDWVEVTVLGLYIKFSPSCEKTRAPFTWEAFQGIGSFMRLAVPSALMVCLEWWSYELLVLLSGMLPNAALETSVLSICISTVILVYNLPYGIGTAASVRVSNELGAGNPDSARLVVVVALSIIIFTAVLVSVTLLSLRHFIGIAFSNEEEVVNYVTRMVPLLSISVITDNLQGVLSGISRGCGWQHLGAYVNLGAFYLVGIPVALVAGFALHLGGAGFWIGMIAGGATQVTLLTIITAMTNWRKMADKARDRVYEGSLPIQAN

SEQ ID NO: 32 - protein, 479 aa, zea mays
MMPGMDEPLLGNGLKTSGKRESLVVAEVRKQMYLAGPLIAAWILQNIVQMISIMFVGHLGELALSSASIATSFAGVTGFSLLSGMASSLDTLCGQSFGAKQYYLLGIYKQRAILVLTLVSLVVAIIWSYTGQILLLFGQDPEIAAGAGSYIRWMIPALFVYGPLQCHVRFLQTQNIVLPVMLSSGATALNHLLVCWLLVYKIGMGNKGAALANAISYFTNVSILAIYVRLAPACRNTWRGFSKEAFHDITSFLRLGIPSALMVCLEWWSFELLVLLSGLLPNPKLETSVLSISLNTGSLAFMIPFGLSAAISTRVSNELGAGRPHAAHLATRVVMVLAIVVGVLIGLAMILVRNIWGYAYSNEKEVVKYISKMMPILAVSFLFDCVQCVLSGVARGCGWQKIGACVNLGAYYLIGIPAAFCFAFMYHLGGMGLWLGIICALVIQMLLLLTITLCSNWEKEALKAKDRVFSTSVPADMMT

SEQ ID NO: 33 - protein, 475 aa, zea mays; identical to SEQ ID NO: 24

SEQ ID NO: 34 - protein, 476 aa, zea mays; identical to SEQ ID NO: 26

SEQ ID NO: 35 - protein, 431 aa, zea mays
MAKKPVEEALLAAADEHEEENLSVREELKKQLWLAGPMIAGALLQNVIQMISVMYVGHLGELPLAGASMANSFATVTGLSLLLGMASALDTLCGQAFGARQYYLLGIYKQRAMFLLTLCHVRFLQTQNIVVPVMASAGAAAACHVVVCWALVYALGMGSKGAALSNAISYWVNVAVLAVYVRVSSACKETWTGFSTEAFRDALGFFRLAVPSALMVCLEMWSFELIVLLSGLLPNPKLETSVLSISLNTAAFVWMIPFGLSSAISTRVSNELGAGRPRAARLAVRVVVLLSVAEGLGVGLILVCVRYVWGHAYSNVEEVVTYVANMMLVIAVSNFFDGIQCVLSGVARGCGWQKIGACINLGAYYIVGIPSAYLIAFVLRVGGTGLWLGIICGLIVQLLLLAIITLCTNWDSEATKAKNRVFNSSSPASGT

SEQ ID NO: 36 - DNA, 1803 bp, Zea mays
ggcgggagagagaagagtcgacgacgacggtaaccggccgccatggaggtgagggtgccgcttcttccgcagcacatattgcggaacggcgacggacgggagaagaagtgcggcgtgaggcggtggagggagctgctagcgcgggaggccgggaaggtcggctgcgtggccctgccgatggcggccgtgagcgtgtcgcagtacgcggtgcaggtggcgtccaacatgatggtcggccccctccccggcgtcctcccgctctccgcctccgccatcgccacctccctcaccaccgtatccggtttcagcctcctcattggtatggcaagtggactggaaactctatgcggtcaagcctatggagcaaaacagtatgataaactggggatgcacacctacagagctatagtcacactcattgctgtgagcattccaatctcacttctgtgggcattcataggcaaactcctgatcctcataggtcaggaccccttgatctcaagggaagctgggagatacatagtctggttgattccaggtctctatgcatatgccatcagccagcctctcacaaaatttctacagtctcagagcctgataattcctatgctttggtcctccattgcaactctgctcttgcacattcctctttgttggttactagtgttcaagaccagtatggggtacattggagcttctttggcaataagcttgtcgtattggttaaacgtgatgatgcttgccgcttacatcagatactcggattcttgtaaggagacccgctcacctccaaccgttgaggccttcaaaggagtcagtgtgtttctacgcctggctctgccgtctgcactgatgttgtgtttcgaatggtggtcttttgagatccttattcttgtctcaggrattttaccaaatccagagctgcamacttcagttctttcaatctgtttgacgactatcacattaatgtatactataccttatgggcttggagcggctgcaagcactagggtagcaaatgagttgggtggtggtaaccctgaaggagctcgatcatctgttcgtgccatcatgtgtattgcagtgatggaagcagctatgatcacagtcatattgttagcgtcacagcacatcttgggttacgcatatagcagcgacaaggatgtcgtcgcgtatgtcaatgcaatggttccccttgtgtgcgtctccgttgctgctgacagcctacaaggtgttctatcaggtgttgcccgaggatgcgggtggcagcacttgggcgcctacgtcaacctgggctcgttctacctcgtcgggattccaacagcactcttcctcggcttcgttctgaagatggaagcgaaagggctttggatgggcatttcctgcggctccatagtgcagttcttgcttcttgccgtcataacgttcttcagcaactgggagaagatgtctgagaaggcaagggagagagttttcagcgatgatgagccgtcagataagggaacttcggattccgatggaccgagttttgttctagttttggcacgagccatctgaatctgatcccccttgtgaagacgcagcggacaagatcttgtagacaaagaagcccatcttggaatggggtttatgatgccttgctttctggtctctgaactgaatgtacagggaacttgttgaactctgaccactgttctgtgctccgtgctcgtgcttgctgttgtttgttaaaacaacgggacaattgtagcagaaacatgactacatgaggcttctgatgctatggtagaaatagtaaccaca

SEQ ID NO: 37 - protein, 504 aa, Zea mays
  misc_feature (295)..(295): Xaa can be any naturally occurring amino acid
MEVRVPLLPQHILRNGDGREKKCGVRRWRELLAREAGKVGCVALPMAAVSVSQYAVQVASNMMVGPLPGVLPLSASAIATSLTTVSGFSLLIGMASGLETLCGQAYGAKQYDKLGMHTYRAIVTLIAVSIPISLLWAFIGKLLILIGQDPLISREAGRYIVWLIPGLYAYAISQPLTKFLQSQSLIIPMLWSSIATLLLHIPLCWLLVFKTSMGYIGASLAISLSYWLNVMMLAAYIRYSDSCKETRSPPTVEAFKGVSVFLRLALPSALMLCFEWWSFEILILVSGILPNPELXTSVLSICLTTITLMYTIPYGLGAAASTRVANELGGGNPEGARSSVRAIMCIAVMEAAMITVILLASQHILGYAYSSDKDVVAYVNAMVPLVCVSVAADSLQGVLSGVARGCGWQHLGAYVNLGSFYLVGIPTALFLGFVLKMEAKGLWMGISCGSIVQFLLLAVITFFSNWEKMSEKARERVFSDDEPSDKGTSDSDGPSFVLVLARAI

SEQ ID NO: 38 - protein, 514 aa, Sorghum bicolor
MEERVPLLPQYTLRNDDGREEKCGGGGGVRWWRELLAREAGKVGCVALPMAAVSVSQYAVQVASNMMVGHLPGVLPLSASAIATSLATVSGFSLLIGMASGLETLCGQAYGAKQYDKLGMHTYRAIVTLIVVSIPISLLWAFIGKLLMLIGQDPLISKEAGRYIAWLIPGLFAYAISQPLTKFLQSQSLIIPMLWSSIATLLLHIPICWLLVFKTSLGYIGASLAISLSYWLNVIMLAAYIRYSNSCKETRSPPTVEAFKGVGVFLRLALPSALMLWFHIGLMNSIPQFYSFEWWSFEILILVSGILPNPELQTSVLSICLTTITLMYTIPYGLGAAASTRVANELGGGNPEGARSSVQVVMCIAVMEAVIITIILLASQHILGYAYSSDKDVVAYVNAMVPFVCVSVAADSLQGVLSGIARGCGWQHLGAYVNLGSFYLVGIPTALFLGFVLKMEAKGLWMGISCGSIVQFLLLAIITFFSNWQKMSEKARERVFSDEPSDKEPLESDGSNLF

SEQ ID NO: 39 - protein, 490 aa, Triticum aestivum
MEGRAPLLPRRQEAAKSGGWRCGAAAAEARKVAHVALPMAAVSVAQYAVQVASNMMVGHLPGGVLALSASAIATSLASVSGFSLLIGMSNGLETLCGQAYGAEQYGRLGVQTYRAMVTLTAVSIPISLLWIFMGKLLTLIGQDPVISHEAGRYIMWLIPALFAYAVSQPLTKFLQSQSLIIPMLWSSIATLLLHIPVCWLLVFKTSLGYIGAALAISVSYWLNVFMLVAYIGCSNSCKETFSPPTLDAFSGVGVFMRLALPSALMLCFEWWSFEVIILLSGLLPNPELQTSVLSTCMTTITLMYTIAYGIGAAGSTRVSNELGAGNPEGARLAVRVVMSIAVTEAVLITGALLASQHILGYAYSSDKEVVDYVNAMVPFICISVAADSLQGVLSGIARGCGSQHLGAYVNLGSFYLFGIPMSLLLGFVLKMGGKGLWMGISCGSIVQFLLLSGIVFFSNWQKMSDNARERVFGGTPAEKEPLMSDVTGAA

SEQ ID NO: 40 - protein, 483 aa, Arabidopsis thaliana
MEDPLLLGDNQIITGSLKPTPTWRMNFTAELKNLSRMALPMATVTVAQYLLPVISVMVAGHRSELQLSGVALATSFTNVSGFSVMFGLAGALETLCGQAYGAKQYAKIGTYTFSAIVSNVPIVVLISILWFYMDKLFVSLGQDPDISKVAGSYAVCLIPALLAQAVQQPLTRFLQTQGLVLPLLYCAITTLLFHIPVCLILVYAFGLGSNGAALAIGLSYWFNVLILALYVRFSSSCEKTRGFVSDDFVLSVKQFFQYGIPSAAMTTIEWSLFEFLILSSGLLPNPKLETSVLSICLTTSSLHYVIPMGIGAAGSIRVSNELGAGNPEVARLAVFAGIFLWFLEATICSTLLFICRDIFGYAFSNSKEVVDYVTELSPLLCISFLVDGFSAVLGGVARGSGWQHIGAWANVVAYYLLGAPVGLFLGFWCHMNGKGLWIGVVVGSTAQGIILAIVTACMSWNEQAAKARQRIVVRTSSFGNGLA

SEQ ID NO: 41 - protein, 476 aa, Arabidopsis thaliana
MEEPFLLQDEHLVPCKDTWKSGQVTVELKKVSSLAAPMAAVTIAQYLLPVISVMVAGHNGELQLSGVALATSFTNVSGFSILFGLAGALETLCGQAYGAKQYEKIGTYTYSATASNIPICVLISVLWIYIEKLLISLGQDPDISRVAGSYALWLIPALFAHAFFIPLTRFLLAQGLVLPLLYCTLTTLLFHIPVCWAFVYAFGLGSNGAAMAISVSFWFYVVILSCYVRYSSSCDKTRVFVSSDFVSCIKQFFHFGVPSAAMVCLEWWLFELLILCSGLLPNPKLETSVLSICLTTASLHYVIPGGVAAAVSTRVSNKLGAGIPQVARVSVLAGLCLWLVESAFFSTLLFTCRNIIGYAFSNSKEVVDYVANLTPLLCLSFILDGFTAVLNGVARGSGWQHIGALNNVVAYYLVGAPVGVYLAFNRELNGKGLWCGVVVGSAVQAIILAFVTASINWKEQAEKARKRMVSSENRLA

SEQ ID NO: 42 - protein, 476 aa, Arabidopsis thaliana
MEEPFLPQDEQIVPCKATWKSGQLNVELKKVSRLAVPMATVTIAQYLLPVISVMVAGHNGELQLSGVALATSFTNVSGFSIMFGLVGSLETLSGQAYGAKQYEKMGTYTYSAISSNIPICVLISILWIYMEKLLISLGQDPDISRVAGSYALRLIPTLFAHAIVLPLTRFLLAQGLVLPLLYFALTTLLFHIAVCWTLVSALGLGSNGAALAISVSFWFFAMTLSCYVRFSSSCEKTRRFVSQDFLSSVKQFFRYGVPSAAMLCLEWWLFELLILCSGLLQNPKLETSVLSICLTTATLHYVIPVGVAAAVSTRVSNKLGAGIPQVARVSVLAGLCLWLVESSFFSILLFAFRNIIGYAFSNSKEVVDYVADLSPLLCLSFVLDGFTAVLNGVARGCGWQHIGALNNVVAYYLVGAPVGIYLAFSCELNGKGLWCGVVVGSAVQAIILAIVTASMNWKEQAKKARKRLISSENGLA

SEQ ID NO: 43 - protein, 476 aa, Arabidopsis thaliana
MEEPFLPRDEQLVSCKSTWQSGQVTVELKKVSRLAAPMATVTIAQYLLPVISVMVAGHIGELELAGVALATSFTNVSGFSIMFGLVGALETLCGQAYGAEQYEKIGTYTYSAMASNIPICFIISILWIYIEKLLITLGQEPDISRVAGSYSLWLVPALFAHAIFLPLTRFLLAQGLVISLLYSAMTTLLFHIAVCWTLVFALGLGSNGAAIAISLSFWFYAVILSCHVRFFSSCEKTRGFVSNDFMSSIKQYFQYGVPSAGLICLEWWLFELLILCSGLLPNPKLETSVLSICLTIGTLHYVIPSGVAAAVSTRVSNKLGAGNPQVARVSVLAGLCLWLVESAFFSTLLFTCRNIIGYTFSNSKEVVDYVADISPLLCLSFILDGLTAVLNGVARGCGWQHIGALINVVAYYLVGAPVGVYLAFSREWNGKGLWCGVMVGSAVQATLLAIVTASMNWKEQAEKARKRIISTENGLV

SEQ ID NO: 44 - protein, 476 aa, Arabidopsis thaliana
MEEPFLLRDELLVPSQVTWHTNPLTVELKRVSRLAAPMATVTIAQYLLPVISVMVAGHNGELQLSGVALANSFTNVTGFSIMCGLVGALETLCGQAYGAKQYEKIGTYAYSAIASNIPICFLISILWLYIEKILISLGQDPEISRIAGSYAFWLIPALFGQAIVIPLSRFLLTQGLVIPLLFTAVTTLLFHVLVCWTLVFLFGLGCNGPAMATSVSFWFYAVILSCYVRFSSSCEKTRGFVSRDFVSSIKQFFQYGIPSAAMICLEWWLFEILILCSGLLPNPKLETSVLSICLTIETLHYVISAGVAAAVSTRVSNNLGAGNPQVARVSVLAGLCLWIVESAFFSILLFTCRNIIGYAFSNSKEVLDYVADLTPLLCLSFILDGFTAVLNGVARGSGWQHIGAWNNTVSYYLVGAPVGIYLAFSRELNGKGLWCGVVVGSTVQATILAIVTASINWKEQAEKARKRIVSTENRLA

SEQ ID NO: 45 - protein, 485 aa, Arabidopsis thaliana
MDSAEKGLLVVSDREEVNKKDGFLRETKKLSYIAGPMIAVNSSMYVLQVISIMMVGHLGELFLSSTAIAVSFCSVTGFSVVFGLASALETLCGQANGAKQYEKLGVHTYTGIVSLFLVCIPLSLLWTYIGDILSLIGQDAMVAQEAGKFATWLIPALFGYATLQPLVRFFQAQSLILPLVMSSVSSLCIHIVLCWSLVFKFGLGSLGAAIAIGVSYWLNVTVLGLYMTFSSSCSKSRATISMSLFEGMGEFFRFGIPSASMICLEWWSFEFLVLLSGILPNPKLEASVLSVCLSTQSSLYQIPESLGAAASTRVANELGAGNPKQARMAVYTAMVITGVESIMVGAIVFGARNVFGYLFSSETEVVDYVKSMAPLLSLSVIFDALHAALSGVARGSGRQDIGAYVNLAAYYLFGIPTAILLAFGFKMRGRGLWIGITVGSCVQAVLLGLIVILTNWKKQARKARERVMGDEYEEKESEEEHEYIS

SEQ ID NO: 46 - protein, 481 aa, Arabidopsis thaliana
MGDAESTKDRLLLPVERVENVTWSDLRDGSFTVELKRLIFFAAPMAAVVIAQFMLQIVSMMMVGHLGNLSLASASLASSFCNVTGFSFIIGLSCALDTLSGQAYGAKLYRKLGVQTYTAMFCLALVCLPLSLIWFNMEKLLLILGQDPSIAHEAGKYATWLIPGLFAYAVLQPLTRYFQNQSLITPLLITSYVVFCIHVPLCWFLVYNSGLGNLGGALAISLSNWLYAIFLGSFMYYSSACSETRAPLSMEIFDGIGEFFKYALPSAAMICLEWWSYELIILLSGLLPNPQLETSVLSVCLQTISTMYSIPLAIAAAASTRISNELGAGNSRAAHIVVYAAMSLAVIDALIVSMSLLIGRNLFGHIFSSDKETIDYVAKMAPLVSISLMLDALQGVLSGIARGCGWQHIGAYINLGAFYLWGIPIAASLAFWIHLKGVGLWIGIQAGAVLQTLLLALVTGCTNWESQADKARNRMALAYGT

SEQ ID NO: 47 - protein, 482 aa, Arabidopsis thaliana
MGDAESTSKTSLLLPVERVENVTWRDLRDGLFTAELKRLICFAAPMAAVVIAQFMLQIISMVMVGHLGNLSLASASLASSFCNVTGFSFIVGLSCALDTLSGQAYGAKLYRKVGVQTYTAMFCLALVCLPLTLIWLNMETLLVFLGQDPSIAHEAGRYAACLIPGLFAYAVLQPLTRYFQNQSMITPLLITSCFVFCLHVPLCWLLVYKSGLGNLGGALALSFSNCLYTIILGSLMCFSSACSETRAPLSMEIFDGIGEFFRYALPSAAMICLEWWSYELIILLSGLLPNPQLETSVLSVCLQTTATVYSIHLAIAAAASTRISNELGAGNSRAANIVVYAAMSLAVVEILILSTSLLVGRNVFGHVFSSDKETIDYVAKMAPLVSISLILDGLQGVLSGIARGCGWQHIGAYINLGAFYLWGIPIAASLAFWIHLKGVGLWIGIQAGAVLQTLLLTLVTGCTNWESQADKARNRMALAYGT

SEQ ID NO: 48 - protein, 487 aa, Arabidopsis thaliana
MEDAESTTKDPVDRVEKVTWRDLQDGSFTAELKKLICFAAPMAAVVITQSMLQIITMVIVGHLGRLSLASASFAISFCNVTGFSFIMGLSCALDTLSGQAYGAKLYRKLGVQAYTAMFCLTLVCLPLSLLWFNMGKLLVILGQDPSIAHEAGRFAAWLIPGLFAYAVLQPLTRYFKNQSLITPLLITSCVVFCLHVPLCWLLVYKSGLDHIGGALALSLSYWLYAIFLGSFMYFSSACSETRAPLTMEIFEGVREFIKYALPSAAMLCLEWWSYELIILLSGLLPNPQLETSVLSVCLQTLSMTYSIPLAIAAAASTRISNELGAGNSRAAHIVVYAAMSLAVVDALMVGTSLLAGKNLLGQVFSSDKNTIDYVAKMAPLVSISLILDSLQGVLSGVASGCGWQHIGAYINFGAFYLWGIPIAASLAFWVHLKGVGLWIGIIAGAVLQTLLLALVTGCINWENQAREARKRMAVAHESELTESELPF

SEQ ID NO: 49 - protein, 487 aa, Arabidopsis thaliana
MQDAERTTNDPVDRIEKVTWRDLQDGSFTAELKRLICFAAPMAAVVIIQFMIQIISMVMVGHLGRLSLASASFAVSFCNVTGFSFIIGLSCALDTLSGQAYGAKLYRKLGVQAYTAMFCLTLVCLPLSLLWFNMGKLIVILGQDPAIAHEAGRYAAWLIPGLFAYAVLQPLIRYFKNQSLITPLLVTSSVVFCIHVPLCWLLVYKSGLGHIGGALALSLSYWLYAIFLGSFMYYSSACSETRAPLTMEIFEGVREFIKYALPSAAMLCLEWWSYELIILLSGLLPNPQLETSVLSICFETLSITYSIPLAIAAAASTRISNELGAGNSRAAHIVVYAAMSLAVMDALMVSMSLLAGRHVFGHVFSSDKKTIEYVAKMAPLVSISIILDSLQGVLSGVASGCGWQHIGAYINFGAFYLWGIPIAASLAFWVHLKGVGLWIGILAGAVLQTLLLALVTGCTNWKTQAREARERMAVAHESELTESELPI

SEQ ID NO: 50 - DNA, 1434 bp, Arabidopsis thaliana
atggaagatccacttttattgggagatgatcagttaatcactagaaacctcaagtcaacgccgacatggtggatgaattttacggcggagctgaagaacgtcagctctatggcggcgcctatggccaccgtgacagtgtctcaatatctattgcccgtgatctcggtcatggtcgccggccactgcggtgaactccagctgtctggtgtcactcttgccactgctttcgcaaacgtctccggcttcggcatcatgtatggtttagtgggtgcacttgaaactctatgtggccaagcttatggagcaaaacaatacactaaaatcggaacttacactttctctgcaatagtctcaaacgtacctatagttgttctcatatcgattctctggttttacatggacaaactctttgtttcacttggacaagatcctgacatctccaaggtagctggttcttacgcggtttgtcttataccggcattgttagctcaagcagtgcaacaacctttgactcggtttctccagactcagggtttggttcttcctcttctctactgtgccataaccacccttttattccatataccagtttgtttgattctggtttacgcgtttggtcttggaagcaatggagccgccttggctattggtttgtcttactggtttaatgtcttgattcttgctttatatgtgagattttcaagcgcttgcgagaagactcgcggctttgtatccgatgatttcgtgttgagtgtcaagcagttttttcagtatgggataccatcagcagcaatgacaaccatagaatggtcgttgtttgagctccttatcttatcttcaggactcctcccaaacccgaaactcgagacctctgttctttccatttgtcttacaacatcatctctccactgtgtcattccaatgggtatcggggctgctggaagcacacggatttcaaacgaattgggagcgggaaatccggaggttgctaggctggcagtgtttgccggtattttcctttggttcctagaggctaccatttgtagcacacttctgttcacttgcaaaaatatttttggctacgcgttcagcaatagcaaagaagttgtggactatgtcacggagctatcttcgctgctttgtctttcatttatggtcgatggattttcttcagtgcttgatggggttgctaggggaagtgggtggcaaaatattggagcttgggcaaatgtggtggcttactatctcctaggagctcctgttggatttttcttaggattttggggtcatatgaacggcaaagggctatggattggtgtgatcgttgggtccactgctcaagggatcatactagctatagtcactgcttgcctgagttgggaggagcaggctgccaaggccagagaaagaatagttggaagaacattggagtga

SEQ ID NO: 51 - protein, 477 aa, Arabidopsis thaliana
MEDPLLLGDDQLITRNLKSTPTWWMNFTAELKNVSSMAAPMATVTVSQYLLPVISVMVAGHCGELQLSGVTLATAFANVSGFGIMYGLVGALETLCGQAYGAKQYTKIGTYTFSAIVSNVPIVVLISILWFYMDKLFVSLGQDPDISKVAGSYAVCLIPALLAQAVQQPLTRFLQTQGLVLPLLYCAITTLLFHIPVCLILVYAFGLGSNGAALAIGLSYWFNVLILALYVRFSSACEKTRGFVSDDFVLSVKQFFQYGIPSAAMTTIEWSLFELLILSSGLLPNPKLETSVLSICLTTSSLHCVIPMGIGAAGSTRISNELGAGNPEVARLAVFAGIFLWFLEATICSTLLFTCKNIFGYAFSNSKEVVDYVTELSSLLCLSFMVDGFSSVLDGVARGSGWQNIGAWANVVAYYLLGAPVGFFLGFWGHMNGKGLWIGVIVGSTAQGIILAIVTACLSWEEQAAKARERIVGRTLE

SEQ ID NO: 52 - protein, 485 aa, Glycine max
MEENLLAKQREKQKVTWDGLGEEMKRIICIAVPMVIVTATQYLLQVVSIMMVGHLNNNLYLSGAALAISLATVTGFSVLAGMASGLETICGQAYGAQQYEKVGVQTYTAIFSLTVVCLPLTFIWISMEKILVFIGQDPLIAQEAGKFLIWLVPALFAHAIMQPFVRYFQMQSLLLPMLISSCVTLCIHIPLCWALVFQTGMNNIGGALAMSISIWLNVTFLGLYMRYSPACAKTRAPISMELFQGIWEFFRFAIPSAVMICLEWWSFELLILLSGLLPNPQLETSVLSICLNTISTLFSIPFGIAAAASTRISNELGAGNPHAAHVAVLAAMSFAIMETAIVSGTLFVCRHDFGYIFSNEKEVVDYVTVMAPLICISVILDSIQGVLAGVARGCGWQHIGVYVNLGAFYLCGIPVAATLAFLAKMRGKGLWIGVQVGAFVQCILFSTITSCINWEQQCLKFFSQSWVWSTNMFHPSAVIIKRIQA

SEQ ID NO: 53 - protein, 480 aa, Glycine max
MEENLLVLAKGSGEEQKVAWEGLGEEMKRMIDIAGPMVVVTASQRLLQVVSVMMVGHLNDDLFLSSAALAISLTAVTGFSFLMGMASGLETICGQAYGAQQHKKIGVQTYTAIFALTFVCLPFTFLWINMEKILVFIGQDPLIAKEAGKFIIWLIPALFAYAILQPLVRYFQMQSLLLPMLMTSCVTLCVHIPLCWVLVFKTRLNNVGGALAMSISTWSNVIFLGLYMRYSPRCAKTRAPISMELFQGLREFFRFAIPSAVMICLEWWSFELIILLSGLLLNPQLETSVLSICLNTTSILYAIPFGIGAAASTRISNELGAGNPHGACVSVLAAISFAIIETTVVSGTLFACRHVFGYVFSNEKEVVDYVTVMAPLVCISVILDNIQGVLAGVARGCGWQHIGVYVNIGAFYLCGIPMAILLSFFAKMRGKGLWIGVQVGSFAQCVLLSTITSCINWEQQTIKARKRLFGSEFSADDRLI

SEQ ID NO: 54 - protein, 454 aa, Glycine max
MKRIIRVAGPMVFVYASQNLLQVVSIMMIGHLNDELFLSGAALAISLATVTGFSLLTGMASGLETICGQAYGARQYQKTGVQTYTAIFSLTCVCLPLTIIWISLENILVFIGQDPLIAHEAGNFIIWLLPALFAYAILQPLVRYFQMQSLLLPMLATSCVTLCLHIPLCWALVFKTELSNVGGALAMSISIWLNVIFLVLYMRYSPACEKTRAPVSMELFQGIWEFFRFAIPSAVMICLEWWSFELLILLSGLLPNPQLETSVLSICLNTISTLYAIAFGIAAAASTRISNELGAGNPHSARVAVLASMSFAIMEATIISGILFVCRHVFGYTFSNKKEVVDYVTVMAPLVCISVILDNIQGVLAGIARGCGWQHIGVYVNLGAFYLCGIPVAASLAFLAKMSGKGLWIGLQVGAFVQCALLSTVTSCTNWEQQAMKARKRLFDSEISAENILV

SEQ ID NO: 55 - protein, 493 aa, Glycine max
EREAEYVMRWSVFGEEMKRVGYLAGPMINVTLSQYFLQIISMMMVGHLGKLVLSSTAIAISLCAVSGFSLIFGMSCALETQCGQAYGAQQYRKFGVQIYTAIVSLTLACLPLTLLWVYLGKILIFLGQDPLISQEAGKFALCMIPALFAYATLQALVRYFLMQSLTSPLFISSSITLCFHVAFCWLLVFKCGFGNLGAAFSIGTSYWLNVVLLGLYMKFSTECEKTRVPISMELFHGIGEFFRCAIPSAGMICLEWWSFELLTLLSGLLPNPELETSVLSICLSVTTTIYTIPEAIGSAASTRVSNALGAGSPQSAQLSVSAAMTLAASAAILVSSIIFACRQVVGYVFSSELDVVDYFTDMVPLLCLSVILDTLHGTLSGIARGCGWQHLGAYVNLGAYYVVGIPIAAMLGFWVQLRGKGLWIGILTGAFCQTVMLSLITSCTNWEKQKLFFQSKKSSILTHAVLFSFEQALLVLVLTEEYSLLECPEIAGN

SEQ ID NO: 56 - protein, 448 aa, Glycine max
MREELKKVGTIAAPMVVASVLQYLLQVVSLVMVGHLNQLSLSSVAIAISLTNVSGFSVLSGMAGGLETLCGQAFGAGQYEKFGQYTYTAVISLSLICFPITILWTFMDKILTLLGQDPTISLEARKYAIWLIPALFGSAILKPLTRFFQTQSLISPMILTSAIALCFHGATCWTLVFKLELGHVGAAISFSLCVWFNVMLLLSFVRYSSACEKTRIPFSKNALVGVGVFFRFAVPAAVMVCLKWWACEILVLLAGLFPNPKLETSVLSIWFVSQSNCMVILFPLANISIEAYTRVSNELGAGNPQAVRVAVSATMFLAVTEGLIVSATLFGCRHLLGYAYSDDRMVVHYVAVMTPLLCLSIFTDSLQGVLSGVARGSGWQHLGAYVNLGAFYLVGIPVGIVLGFVAHLRAKGLWIGIVTGSIVQSILLSLVTALTNWKKQKYCMFGVN

SEQ ID NO: 57 - protein, 475 aa, Glycine max
MEESLVKKHEQERVTWGVYSEEMRRVCHIAGPMVAVVSSQYLLQVVSTMIVGHLGELYLSSAALAISLSGVTGFSLLMGMASGLETICGQAYGGQQYQRIGIQTYTAIFSLILVSIPVSLLWINMETILVFIGQDPLISHEAGKFTIWLVPALFAYAILQPLVRYFQIQSLLLPMFASSCVTLIIHVPLCWALVFKTSLSNVGGALAVSISIWSNVIFLVLYMRYSSACAKTRAPISMELFKGMWEFFRFAIPSAVMVCLEWWSYELLVLLSGLLPNPQLETSVLSVCLNTIATLYTIPFGIGAAASTRVSNELGAGNSHAARVAVLAAMSLAVIETSIVSATLFACRNVFGYIFSNEKEVVDYVTAMAPLVCISVILDSIQGVLTGIARGCGWQHLGVYVNLGAFYLCGIPMAALLAFLVRLGGKGLWIGIQSGAFVQCILLSIITGCINWEKQAIKARKRLFDEKISADNILV

SEQ ID NO: 58 - protein, 480 aa, Glycine max
MEEGSETGKWGWMKRRRAMREELKKVGTIAAPMVVASVLQYLLQVVSLVMVGHLNQLSLSTVAIATSLTNVSGFSVLSGMAGGLETLGGQAFGAGQYEKFGQYTYTAVISLSLICFPITILWTFMDKILTLLGQDPTISLEARKYAIWLIPALFGSAILKPLTRFFQTQSLISPMILTSAIALCFHGATCWTLVFKLELGHVGAAISFSLCVWFNVMLLLSFVRYSSACEKTRIPFSKNALVGVGDFFRFAVPAAVMVCLKWWACEILVLLAGLFPNPKLETSVLSICLTISTLHFTIPYGFGAAASTRVSNELGAGNPQAVRVAVSATMFLAVTEGLIVSATLFGCRHILGYAYSDDRMVVHYVAVMTPLLCLSIFTDSLQGVLSGVARGSGWQHLGAYVNLGAFYLVGIPVGIVLGFVAHLRAKGLWIGIVTGSIVQSILLSLVTALTNWKKQAMMARERIFDVKPPDENESNHMTSA

SEQ ID NO: 59 - protein, 500 aa, Oryza sativa
MEERIPLLSKRFPADGTAGVGGGREEEGGDRWWSGLAREAGKVGSMALPMAAMSVAQNAVQVASNMMVGHLPGVLPLSASAIATSLASVSGFSLLVGMASGLETLCGQAYGAKQYDKLGVQTYRAIVTLTVVTIPISLLWVFIGKLLTLIGQDPVISHEAGRYIVWLIPGLFAYAVCQPLTKFLQSQSLIFPMLWSSIATLLLHIPLSWLLVFKTSMGFTGAALAISISYWLNTFMLAAYIRFSCSCKVTRSPPTIEAFRGVGLFLRIALPSALMLCFEWWSFEILVLLSGLLPNPELESSVLSICLTTTSLMYTIPYGLGGAASTRVANELGAGNPEGARSAVHLVMSIAGTEAVLVTGMLFAAQRILGYAYSSDEEVVTYFTSMVPFVCISVAADSLQGVLSGIARGCGWQHLGAYVNLGSFYLVGIPVALLLGFGFKMEGKGLWLGIACGSVLQFLLLAVIAFFSNWQKMAEKARERIFGETPSEKQHLVLDATNSV

SEQ ID NO: 60 - protein, 491 aa, Oryza sativa
MAAAAREEQPLLLRREEGEEEGEEVGWRRRWGSEAGKLAYLALPMVAVSLTNYAVQVFSNMMVGHLPGVLPLSSAAIATSLASVTGFSLLIGMASALETLCGQAYGAKQYHTLGVHTYRAILTLLVVCIPLSLLWVFMGKILVLIGQDPLISHGAGRYIVWLIPGLFANALIQPITKFLQSQSLIMPMLVASVATLVFHIPLCWLMVFKTGLGYTGAALSISISYWLNVAMLVAYILLSSSCKETRTPPTIEAFKGLDGFLRLALPSALMICLEWWSFELLILMSGLLPNPELQTSVLSICLTSITLLFTIPYGLGAGGSTRVANELGAGNPEGARSAVYVVLSVAVTEALIVCGTLLASRRLLGRAYSSEEEVISFVAMMVPLVCITVVTDGLQGVMSGIARGCGWQHLGAYVNLGSFYLLGIPMAILLGFVLHMGAKGLWMGIVCGSISQITLLSAITFFTNWQKMAENARERVFSEKPTEPSRYHLVE

SEQ ID NO: 61 - protein, 502 aa, Oryza sativa
MGSSDSQAPLLLPRGSHRKEEEEEEYAAAGKVRGCCGGDGEGGWWREATAEAGRLASLAAPMIAVALLQLMMQLISTVMVGHLGEVALAGAAIANSLTNVSGFSVLMGLACGLETICGQAYGAEQYHKLALYMYRSIIVLLVVSVPIAIIWVFIPEVLPLIGQQPEIASEVGKYALWLIPGLFAFTVAQCLSKFLQTQSLIFPMVLSSSITLALFIPLCWFMVYKVGMGNAGAALSVSICDWVEVTVLGLYIVLSPSCEKTRAPLTWEAFSGIGSFLRLAVPSALMICLEWWSYELLVLLSGILPNPALETSVLSICISTVVLVYNLPHGIGTAASVRVSNELGAGNPEGARLVVGVALSVILCSAVLVSVTLLALRHFIGIAFSNEEEVINYVTRMVPVLSISVITDSLQGVLSGVSRGCGWQHLGAYVNLGAFYLVGVPVALFFGFAMHLGGMGFWMGMVAGGATQVTLLSIITAMTNWRKMAEKARDRVFEERIPTQSV

SEQ ID NO: 62 - protein, 489 aa, Sorghum bicolor
MAAAREEDEATQARPLLLPRRPAQEDQKWWRRWAREAGWVGYLALPMVVVNLSQYAVQVSSNMMVGHLPGVLPLSSAAIATSLANVSGFSLLIGLASALETLCGQAYGAKQYHKLGLDTYRAIVTLLVVCIPLSLLWVFMDKILVLIGQDPLISQGAGRYMIWMIPGLFANAVIQPLTKFLQTQSLIYPLLLSSVATAAIHIPLCYVMVFKTGLGYTGAALTISISYWLNVAMLVGYIVFSSSCKETRARPTIEVFRGVDAFLRLALPSALMMCFEWWSFELLTLMSGLLPNPELQTSVLSICLTSVTLLFTIPFGLGAAGSTRVANELGAGNPDGARSAVRVVLSMAGIDAVIVSGTLLAARRLVGLAYSSEEEVISSVAAMVPLVCITVITDCLQGVLSGVARGCGWQHLGAYVNLGSFYLLGIPMAILLGFVLHMGSRGLWMGIVCGSLSQTTLMSAITFFTDWPKMAEKARERVFSDKAHESAGP

SEQ ID NO: 63 - protein, 514 aa, Sorghum bicolor; identical to SEQ ID NO: 38

SEQ ID NO: 64 - protein, 505 aa, Sorghum bicolor; identical to SEQ ID NO: 30

SEQ ID NO: 65 - protein, 488 aa, Hordeum vulgare
MDSSSEAPLLLSRGNSHKEVPHEAGGKRQRWWREAAEESGRLAALAAPMIAVALLQLMMQLISTVMVGHLGEVALAGAAIANSLTNVSGFSVLIGLACGLETICGQAYGAEQYHKLSLYTYRSIIVLLIVSVPIAIVWVFIPEVLPLIGQQPEIANEAGKYALWLIPGLFAFSVAQCFSKFLQCQSLIFPMVLSSMITLAVFIPLCWFMVYKVGMGNAGAALSVSICDWVEVIVLGLYIKFSPSCEKTRAPLTWEAFKGIGSFMRLAVPSALMICLEWWSYELLVLLSGILPNPALETSVLSICISTVVLLYNLPYGIGTAASVRVSNELGAGNPEGARLVVGVALSIVVCSAALVSTTLLASRHFIGIAFSNEEEVIDYVTRMVPVLSISVITDSLQGVLSGVSRGCGWQHLGAYVNLGAFYLVGIPVALFFGFTMQLRGMGFWIGMIAGGATQVTLLSVITATTKWDKMADKAKERVFEDRLPTQQ

SEQ ID NO: 66 - DNA, 1627 bp, Triticum aestivum
ttcccattccatttccacacccacctcccttcgcctggtccgggggcgggcatccatggactcttcctccgaggcgccgctgctgctcagcagggggaacgagcacaaggaggtgcccgatgtggcgggaggcaagcggccgtggtggagggaggcggcggaggagtccggccggctggccgcgctggctgcgccgatgatcgcggtggcgctgctgcagctgttgatgcagctcatctccaccgtcatggtggggcacctcggcgaggtcgcgctcgctggcgccgccatcgccaactccctcaccaacgtctccggcttcagcgtcctcataggactggcatgcggattggaaactatttgtgggcaggcctatggagcagaacagtatcataagttatccttgtatgcctacaggtccatcattgtacttcttattgtgagtgtgcccatcgccattgtatgggttttcattccggaagtacttcctctcataggtcagcaaccagaaattgcaaatgaggctgggaaatatgcattgtggcttatccctggtttatttgccttcagtgttgctcaatgcttttcaaagtttctgcagtgtcagagcctcatctttcctatggttcttagctccatgatcacactcgctgtatttattcctctgtgttggttcctggtttataaagttggtatgggtaatgttggagctgctttatccgtcagcatctgcgattgggttgaagtgactgttcttggtctttacatcaagttctcaccttcttgtgagaaaacacgtgctccactcacgtgggaagcttttaaaggaattggcagtttcatgcgtttggctgtaccgtcggctcttatgatttgccttgaatggtggtcttacgagctgcttgttctgctttctgggatcttaccaaatccagcacttgaaacttctgtgctttctatatgcatatctacagtggtgctgttgtacaatcttccttacggtattggaacagctgcaagtgtgcgcgtctcaaatgaactaggtgctggcaacccagaaggtgctcgcttggtggtaggtgttgctttgtcgattgtagtttgttcagcaatcctggtgagcacagctcttctagccctgcgccacttcattggaattgcattcagcaacgaggaggaggttgtagattatgtcaccagaatggtacccgtactttccatttcagtcattacagacagcttccaaggagtcctttcaggtgtttctcggggctgtggatggcagcatttaggcgcatatgtcaacctgggtgcattctatcttgttgggatccccgttgcactcttttttggttttacaatgcaactaagaggaatgggattttggattggcatgatagctggtggagccacacaggtcactctcctatctgtgataactgccactacaaaatgggacaagatggctgacaaggctaaggagagagtatttgaagacaggcttccaacacaataagacttgaagaactgccatgtcaaaattttgaagagctaaataatcagagaattctaattacctttaatttcaaaaaaaaaaaaaaaaaaaaaaaaaaaaaaaaaaaaaaaa

SEQ ID NO: 67 - protein, 486 aa, Triticum aestivum
MGSSEAPLLLAHPGEGKEDPGADVGDRRRLRCCWWWRRCGGASSEGWWAEVTAEAGRLAALAAPMIAVALLQLMMQLISTIMVGHLGEVPLAGAAIANSLTNVSGFSVLMGLASGLETICGQAFGAEQYHKVALYTYRSIIVLLIASVPMAITWVFIPDVLPLIGQDPQIASEAGRYALWLIPGLFAFSVAQCLSKFLQSQSLIFPMVLSSFTTLAVFIPLCWFMVYKVGMGNAGAAFAVSICDWVEVTVLGLYIKFSPSCEKTRAPFTWEAFRGIGNFMRLAVPSALMICLEWWSYELLVLLCGVLPNAALETSVLSICISTVVLVYNLPYGIGTAASVRVSNELGAGNPDGARLVVVVALSIIICTAVLLSITLLSFRHFVGIAFSNEEEVVNHVTRMVPLLSISVLTDNLQGVLSGISRGCGWQHLGAYVNLGAFYLIGIPVGLVAGFALHLGGAGFWIGMIAGGATQVTLLSVITAMTNWQK

SEQ ID NO: 68 - DNA, 1633 bp, Eragrostis nindensis
cggcgcccatgatcgcggtggcgctgctgcagctcacgatgcagctcatctccaccgtcatggtgggccacctcggcgaggtcccgctcgcgggggccgccatcgccaactcgctcaccaacgtcgccggattcagcgtcctcattggactagcaaccgggttggaaactctttgtggacaggcctacggaggagaacagtatcataagctatctttgtatacctaccggtctatagttgtacttcttatcgcgagtgtgcccatcgccattatgtgggttttcatcccggaggtacttcctctaattggtcaggatccacagatagccagtgaggctgggaagtatgccttgtggcttatccctggtttatttgccttcagtgtggcacaatgcttttctaaattcctccaatctcagagcctgattttcccaatggttttgagctccttgattacactcactgtcttcattccgttgtgttggttcatgatttataaagttgggatgggtaatgctggagctgctttatcagtcagcatctgtgattgggttgaagtgactgttcttggtctttacatcaaatactcaccttcttgtgagaaaacacgtgctccattcacctgggatgctttcagagggattggcagcttcatgcggttggctgtaccatcagctgttatgctttgtcttgaatggtggtcatacgagcttcttgttctgctttctgggatcttaccaaatccagcacttgaaacttctgtgctttctatatgcatatctacagttgtgttggtatacaatctcccatttggcatcggaacagctgcaagcgtgcgtgtttcaaatgagctaggtgcagggaacccagaaggtgcccgtttagtggtagttgtcgccttatccattgttgtttgctcagctatcctggtgagcatgactcttctatcgttgcgccgttttattggaattgctttcagcaacgaggaggaggttataaattatgtcaccagaatggttccgctgctttcaatttcagttcttgcagacaatcttcaaggtgttctcacaggtatctctaggggctgtggatggcagcatttaggcgcctatgttaaccttggcgcgttctatcttatcggtgttcctgtgggtgttgttctcggttttagatttcatctaggaggagctgggttttggatgggcatgatagctggtggtgccactcaggtcgctctcctatctatcatcactgcaatgacaaactggaggaagatgtcagacaaggctagagagagagtgtttgatgaaaggcttccaactcaggcagcatgattgaatcaacttacagttgcatctgattcgtctcgaaagttttgccacactgtaggtgatttttgtgtacgaggtgctcattttccacatatctgtaatgaagacttatcctctgctcaaaggggctgtccaatgaagctagattagtgtattgctccacggtcttaggctctgttttgatacaaagtatttctagagttttgaaagaatactacagtttagcaaaatacggttttgagaggtatggaggtgtttggatggaacaagttttgcagttttaaaaaccatggtattgctaa

SEQ ID NO: 69 - protein, 443 aa, Eragrostis nindensis
APMIAVALLQLTMQLISTVMVGHLGEVPLAGAAIANSLTNVAGFSVLIGLATGLETLCGQAYGGEQYHKLSLYTYRSIVVLLIASVPIAIMWVFIPEVLPLIGQDPQIASEAGKYALWLIPGLFAFSVAQCFSKFLQSQSLIFPMVLSSLITLTVFIPLCWFMIYKVGMGNAGAALSVSICDWVEVTVLGLYIKYSPSCEKTRAPFTWDAFRGIGSFMRLAVPSAVMLCLEWWSYELLVLLSGILPNPALETSVLSICISTVVLVYNLPFGIGTAASVRVSNELGAGNPEGARLVVVVALSIVVCSAILVSMTLLSLRRFIGIAFSNEEEVINYVTRMVPLLSISVLADNLQGVLTGISRGCGWQHLGAYVNLGAFYLIGVPVGVVLGFRFHLGGAGFWMGMIAGGATQVALLSIITAMTNWRKMSDKARERVFDERLPTQAA

SEQ ID NO: 70 - DNA, 1305 bp, Eragrostis nindensis
cctccctcgccaccgtctccgggttcagcctcctcgttggaatggcaagtgcattggagactctatgtggccaagcttacggtgcaaagcagtaccataagctaggagtgcaaacttacagagcaatagtcaccctcctagtggtctgcatccccatcacagttctctgggcgttcatgggcaaaatcctggttctcatagggcaggaccccttgatcgcccagggagccgggagatacatagtctggctgatcccggggctcttcgcaaacgctgtgttgcagccaatcaccaagttcctgcagacccagagccttatatttgccccgctcgtctcatcagttgcaacgctggcgatccacgtccctctgtgctatatgatggtgttcagaactgggtttgggtataccggagctgctctgtcgataagcatatcctattggctgaatgtgattatgcttgttgtgtacattgcgatgtcaagttcttgcaaggagacacgcacacctccaacgatcgaggccttcaaggaaattgatgcgttcctgcgtctagccctgccctctgcactgatgatctgtcttgaatggtggtcatttgagatccttatccttctctcagggtttctacccaacccagagcttcagacctcggtgctttcaatctgtctatcaagcattacattactctacactctaccatatggatttggagctgctggaagcacaagagtagcaaatgaactgggtgcggggaaccctgaaggagctcgattctctgtccgtgttgtgatgtccatggcggcgatggaggcggttatcatcagtggaactcttttagctttacgacgtcttgtcggtcaggcatacagcagtgaggaggaggtggtatcattcgtcgcaaccatggttcctttggttagcatcactgtgattacagatggccttcaaggagttctctcaggcattgctcgaggatgcggttggcaacacctgggcgcgtacgtgaacctcggctcgttctacctgctggggatgccgatggcgatcctcctcggcttcgtgctgaacatgggaggcagagggctctggatgggcgtcctctgcggttccgtatcacagactacgctcctgtccgccatcacgatattcactgactggcccaagatggcagagaaagccaggcagagggtgttcgatgagaagccggcggatcccgagtcgagacacctgctggaatagcttgactccagattcaaacgattcagttatctgattctgcaggggctgcagaattcgttgatga

SEQ ID NO: 71 - protein, 412 aa, Eragrostis nindensis
SLATVSGFSLLVGMASALETLCGQAYGAKQYHKLGVQTYRAIVTLLVVCIPITVLWAFMGKILVLIGQDPLIAQGAGRYIVWLIPGLFANAVLQPITKFLQTQSLIFAPLVSSVATLAIHVPLCYMMVFRTGFGYTGAALSISISYWLNVIMLVVYIAMSSSCKETRTPPTIEAFKEIDAFLRLALPSALMICLEWWSFEILILLSGFLPNPELQTSVLSICLSSITLLYTLPYGFGAAGSTRVANELGAGNPEGARFSVRVVMSMAAMEAVIISGTLLALRRLVGQAYSSEEEVVSFVATMVPLVSITVITDGLQGVLSGIARGCGWQHLGAYVNLGSFYLLGMPMAILLGFVLNMGGRGLWMGVLCGSVSQTTLLSAITIFTDWPKMAEKARQRVFDEKPADPESRHLLE

SEQ ID NO: 72 - DNA, 1520 bp, Eragrostis nindensis
gagcctgtcgcagtacgcggtgcaggtggcgtccaacatgatggtcgggcacctccccggcgtcctcccgctctccgcctccgccatcgccacctccctcgccaccgtctccggcttcagcctcctcattggaatggcaagtggattggaaactctatgcggccaagcgtacggggctaaacagtatgacaaattgggactgcaaacatacagagctatagtcacactcctagttgtgagcattccaatctcactattgtgggccttcataggcaaactcctgatcctcataggacaagatccattgatctcaaaggaagctgggagatacatagcctggttgattccagggctctttgcgtatgcagtcagccaacctctcacaaaatttctgcagtcccagagtctcataattcctatgctttggtcctccatagcgacactgctcttgcacatccctctttgttggttgttggttttcaagacaagcctggggtttattggagcttctttagcgataagcttatcatattggctgaatgtgatcatgcttgctgcttacatccgatactcaagtgcttgtaaagagacccgctcgcctcctactgttgaggccttcaaaggagttggtttgttcctgcgcttggctctgccatctgcactaatgttgtgtttcgaatggtggtcctttgaggttcttattcttgtctctggacttctgcccaatccagagctgcaaacttcagtcctctcgatttgcctaacgactatcacgttgatgtatactataccttatggacttggagcggcagcaagcacgcgagtggccaatgaattaggtgctagcaaccctgaaggagctcgatcggctgttcgaattgttatgactatcgcagcgctagaggcaggtctcgttacaatttcattgttagcatcacaacacatcgtgggctatgcatatagcagcgataaggaggtggtcgcttatgtcaatgcaatggttccctttgtgtgcgtctcagttgctgctgatagccttcaaggagttctctcaggtattgcccgaggaagcgggtggcagcacttgggtgcctatgtgaacctcggttcgttctatctgattgggattccagtggcactcttcctcggctttgttctgaagatggaaggaaaagggctttggatgggcatttcctgcggttccgtagtgcagttcttattactcgcagtgataacattcttcagcaactggcaaaagatgtctgagaaagcaagggaaagggtgttcagcgaagagctgtctaataaggaaccattggaatcggatggaccatctttgatctaattttaacatgcgtcgactaatctcctgacctgaatgtgaagaagcagttgttgtacattagtacagaataatcttttcgatatataagaggatacgaactgtggatgcatgtgatgtgtgcacgttgtaactttcattttactaaaaaatagttggaagagatcggaagagcgtc

SEQ ID NO: 73 - protein, 447 aa, Eragrostis nindensis
SLSQYAVQVASNMMVGHLPGVLPLSASAIATSLATVSGFSLLIGMASGLETLCGQAYGAKQYDKLGLQTYRAIVTLLVVSIPISLLWAFIGKLLILIGQDPLISKEAGRYIAWLIPGLFAYAVSQPLTKFLQSQSLIIPMLWSSIATLLLHIPLCWLLVFKTSLGFIGASLAISLSYWLNVIMLAAYIRYSSACKETRSPPTVEAFKGVGLFLRLALPSALMLCFEWWSFEVLILVSGLLPNPELQTSVLSICLTTITLMYTIPYGLGAAASTRVANELGASNPEGARSAVRIVMTIAALEAGLVTISLLASQHIVGYAYSSDKEVVAYVNAMVPFVCVSVAADSLQGVLSGIARGSGWQHLGAYVNLGSFYLIGIPVALFLGFVLKMEGKGLWMGISCGSVVQFLLLAVITFFSNWQKMSEKARERVFSEELSNKEPLESDGPSLI

SEQ ID NO: 74 - DNA, 653 bp, Paspalum notatum
ccagccgccggacactccaatcgcatacacgaacacgactcctcggaacgcggctcgatcgacgcgacaacggcagccgccatggaggagagggtgccgcttctgcagcagcagtggacgctgcgagacggcggcgacggaccggaggagaagcgcggcggcctgaggtggtggtggtggagggaccttgcgcgggaggccgggaagttcgggtacgtcgccctgccgatggcggccgtgagcgtgtcgcagtcggcggtgcaggtggcgtccaacatgatggtcggccacctccccggcgtcctcccgctttcggcctccgctatcgcaacctccctcgccaccgtctccggtttcagcctcctcattggaatggcaagtggcttggaaactctatgtggtcaagcctacggggcgaaacagtacgataaattggggatgcacacctatagggccatagtcacgctcatagttgtgagcattccaatctcccttctctgggtattcataggcaaactcctgatcctcataggccaagaccctttgatctcaaaggaagctgggagatatatagtctggttgattccaggactctttgcatatgcaatcagccagcctctcacgaaatttctgcagtctcagagtctgataat

SEQ ID NO: 75 - protein, 501 aa, Paspalum notatum
  misc_feature (191)..(192): Xaa can be any naturally occurring amino acid
MEERVPLLQQQWTLRDGGDGPEEKRGGLRWWWWRDLAREAGKFGYVALPMAAVSVSQSAVQVASNMMVGHLPGVLPLSASAIATSLATVSGFSLLIGMASGLETLCGQAYGAKQYDKLGMHTYRAIVTLIVVSIPISLLWVFIGKLLILIGQDPLISKEAGRYIVWLIPGLFAYAISQPLTKFLQSQSLIXXPMLWSSIATLLLHIPLCWLLVFKTSLGYIGASLAISLSYWLNVIMLAAYIIFSNSCKETRSPPTIEAFKGVGIFLRLALPSALMLCFEWWSFEILILLSGILPNPELQTSVLSICLTTITLTYTIPYGLGAAASTRVANELGGGNPEGARSAVRVVMCIAVIGATIVTIILLSSQHILGYAFSSDKEVVAYVNAMVPFVCVSVAADSLQGVLSGXXXGCGWQHLGAYVNLGSFYLVGIPTALLLGFVLKMEGKGLWMGISCGSIVQFLLLAIITFFSNWQKMSDKARERVFNDEQSNKEPLESDGSGSV

SEQ ID NO: 76 - DNA, 1355 bp, Paspalum notatum
gggcgctcgaggcggggtgggtggggtatctggcgctgcccatggtggtggtgaacctgtcgcagtacgccgtgcaggtgtcttccaacatgatggtcgggcacctccccggcgtgctcccgctctcctccaccgccatcgccacatccctcgccaacgtctccggcttcagcctcctgattggaatggcaagtgcactggagacgctatgtggccaagcctacggcgcaaaacagtaccacaaactaggtgtagatacttatagagcagtagtcaccctccttgtggtctgcatccctctctcgcttctctgggtgttcatggataaaatcctggtcgtcatagggcaagaccccctcatctcccaaggagctggaaggtacatgacctggctgatccccgggctcttcgcgaatgcggtgatccagccggtcaccaagttcctgcagacgcagagcctcatataccccttgctgctgtcgtctgtggcgacgatggcgatccacatccctctatgctacttgatggtgttcaagactggctttgggtacacaggtgctgctttgactataggcatatcatactggctgaatgtaggcatgcttgttgggtacatcatgttctcaaattcttgcaaggagacacgcacacgcccaacgattgaagccttcaagggagtcgatgcgttcctgcgtctagccctgccctctgcactaatgatgtgttttgaatggtggtcatttgagctccttattctcttgtcagggttcctacccaacccagagctgcagacctcagtgctttcaatttgtctcacaagtataacgttactcttcaatataccctttggacttggggctgctggaagcacccgagtagcaaatgaactgggtgctgggaaccctgatggagctcgatctgcagtccgcgttgtgctgtccatgacggcgatcgacgcagttatcgtgggcggaactctgctagcggcacggcgcctcgtgggcatggcttacagcagcgaggaggaggtcatatcttctgttgccaccatggttcctctggtctgcatcactgtggtaactgactgtgtacagggagttctctcaggcgttgcccgagggtgcgggtggcagcacctgggcgcttatgtcaatctcggctcattctacctgctggggatcccgatggcgatccttctcggcttcgtgctgcacctggggtcgagagggctctggatgggcatcgtctgcggctctttgtctcagacaatactcatgtccgtcatcacattcttcaccaactggcccaagatggctgacaaggccagggagagggtgttcagcgagaagccaccg

SEQ ID NO: 77 - protein, 451 aa, Paspalum notatum
ALEAGWVGYLALPMVVVNLSQYAVQVSSNMMVGHLPGVLPLSSTAIATSLANVSGFSLLIGMASALETLCGQAYGAKQYHKLGVDTYRAVVTLLVVCIPLSLLWVFMDKILVVIGQDPLISQGAGRYMTWLIPGLFANAVIQPVTKFLQTQSLIYPLLLSSVATMAIHIPLCYLMVFKTGFGYTGAALTIGISYWLNVGMLVGYIMFSNSCKETRTRPTIEAFKGVDAFLRLALPSALMMCFEWWSFELLILLSGFLPNPELQTSVLSICLTSITLLFNIPFGLGAAGSTRVANELGAGNPDGARSAVRVVLSMTAIDAVIVGGTLLAARRLVGMAYSSEEEVISSVATMVPLVCITVVTDCVQGVLSGVARGCGWQHLGAYVNLGSFYLLGIPMAILLGFVLHLGSRGLWMGIVCGSLSQTILMSVITFFTNWPKMADKARERVFSEKPP

SEQ ID NO: 78 - DNA, 1375 bp, Paspalum notatum
gctgctgcagctcacgatgcagctcatctccaccgtcatggtggggcacctcggcgagatgccgctcgccggcgccgccatcgccaactcgctcaccaacgtctccggattcagcgtgcttatgggactggcatgcggattggaaactatttgtggacaggcctacggggcagaacagtatcataagctagctttatttacctacaggtctataattgtactccttatactgtgtgtgcccattgccattatatgggttttcatcccagacatactccctctcataggtcaggatccgcaagtagcaagtgaggctgggaagtatgccttatggcttatccctggtttatttgccttcagtgtggctcagtgcctttcaaagttcctccagtctcagagcttaattttccccttggttttgagctccttaacaacactcactctctttattcctttgtgctggttcatggtttacaaagttgggatgggtaatgctggagctgctttagcagtcagcatctgtgattgggttgaaatcatggtccttggtctttatattaagctctcaccttcgtgtgagaaaactcgtgctccattcacgcgggaagcttttcgagggattgggagtttcatgcggttggcagtaccttcagcacttatgatatgcattgaatggtggtcgtacgagctgcttgttctgctttcagggatcttaccaaatccagcacttgaaacttcggtactatctatatgcatgtctaccatattgttggtgtacaatatcccatatggcatcggagcagctgcaagtgtgcgtgtgtccaatgagctaggtgcagggaacccagatggtgcccgcttagtagtagttgtcgccttatccaccataatttgttcagcagttctggtgagcgtaactcttctatcactgcgccatttcatcggaattgctttcagcaatgaagaggaggttgtagactatgtcaccagaatggtaccgttgctttcaatttcagttcttaatgacaacctccaaggagtcctttcaggtatttctagaggctgtggatggcagaatttaggagcatatgttaacctgggcgcgttctatcttgttggcattcctgtggcacttgttttcggttttgcattgcatctaggaggagctgggttctggattggcataatagctggtggagttacacaggtcactctcctatcagtcatcactgcaatgacaaactgggggaagatggttgatatggctagggatagggtatatgagggaagtcttccaacacaggtagattgaatgcgccaactttaagtcatattggattggattctattaatcttcacctacgttaaatagccg

SEQ ID NO: 79 - protein, 436 aa, Paspalum notatum
LLQLTMQLISTVMVGHLGEMPLAGAAIANSLTNVSGFSVLMGLACGLETICGQAYGAEQYHKLALFTYRSIIVLLILCVPIAIIWVFIPDILPLIGQDPQVASEAGKYALWLIPGLFAFSVAQCLSKFLQSQSLIFPLVLSSLTTLTLFIPLCWFMVYKVGMGNAGAALAVSICDWVEIMVLGLYIKLSPSCEKTRAPFTREAFRGIGSFMRLAVPSALMICIEWWSYELLVLLSGILPNPALETSVLSICMSTILLVYNIPYGIGAAASVRVSNELGAGNPDGARLVVVVALSTIICSAVLVSVTLLSLRHFIGIAFSNEEEVVDYVTRMVPLLSISVLNDNLQGVLSGISRGCGWQNLGAYVNLGAFYLVGIPVALVFGFALHLGGAGFWIGIIAGGVTQVTLLSVITAMTNWGKMVDMARDRVYEGSLPTQVD

SEQ ID NO: 80 - DNA, 1960 bp, Paspalum notatum
cgccccacggtgggggcggaggcgcggcggcaggtggggctcgcggcgccgctggtggcgtgcagcctgctgcagtacagcttgcaggtggtctccgtcatgttcgccggccacctcggggagctctccctctccggcgcctccgtcgctgcctccttcgccaacgtcaccggcttcagcgtcctgctgggcatggggagcgcattggataccttttgtggacaatcatatggagcaaggcaatatgatctgctcgggacacacacacaaagggctatagttgttcttatgcttacaggtgttcctttggcatttgttttggccttctctggtcaaatcctggttgctcttggtcaaaatcctgaaatatcatttcaagctggactgtacgctcagtggttgatccctggtcttttcgcatacggtttgcttcagtgccttaccagatttctgcagacccaaaatattgtccgaatattggtagtttcctctggacttactttgctacttcacattatgctgtgctggttcctggttcaaagttttggccttggccacaaaggcgcagctctggcgacctcaatatcttactggttcaatgtggcattgctagcaatatatgtgaaagcctctgaagctggcagaagaagctggcatggatggtcaagggaggcactaaagttaaaggatgtaaaactatatctatggttggccattccatctacatttatgacctgcttggagtattgggcatttgagatggtggttctcctagcaggatttcttccagatccaaaactggaaacttcaattttatccatcagcctaaacacaatgtggatggtttatacaattccaagtggcctcagtagtgcaataagtattagagtgtccaatgaactaggtgctgggaacccacaggcagcacgcctgtcagtttatatttcaggaatcatgtgcctaactgaaggcctttttgtagctatcatcacagtattagtgcgagatatctggggttatttgtacagcaatgaagaaaaggtcgtgaagcatgtatcgatgatgatgccaattcttgctacttctgacttcatggatggaatacagtgcacactatcaggcgcggctcgaggatgtggctggcagaaagtatgctcacttatcaacctgtttgcttactatgttattggtctcccttcagctgtcacttttgcatttgttttgaagattggtggtaagggcctttggctgggaattatatgtgctatggcagtgcaaatatttgctttggtagtgatgatgcttcgaaccagctggaatgaagaggctgaaaaggcccgggctagagttcagtgttcagatggcagcattacattggactgaatcttatcagttgtgaattgtgattatagatcccttaaggtgcagtgtataggtttatatgagtcctttgggccaaggcagcagctctggatctggcgggactgagctatcagcctaaaagcaggagtacttggcttgcaattgatggccttggagtacgtttctttctggtttctgcggctcaatgatagcatcgtgaaaggcgaggaagcaaccgaaatttggtagctccggaagtggaggttgtcgtggacgctgagctgtttccaaatcccaaatactccacctgctcaccgtgtgggttaatcgactctcacctgaaagatatttgctattactacaattgatgtttgctctgaaaggaaggaaggagagagggtgtaaacttttcatgctactacgcttcaggagttcaggtgtcagtctgtcagggtgtttccctggaatgaaagcttcagggtgcgctcaggatgccatgtcagctagcggtctgttgcgtgggcgttttatacagagctgatcgatggcatctgcgatcgcgtctgtatagataatgtatggatggctcagaccatcc

SEQ ID NO: 81 - protein, 460 aa, Paspalum notatum
RPTVGAEARRQVGLAAPLVACSLLQYSLQVVSVMFAGHLGELSLSGASVAASFANVTGFSVLLGMGSALDTFCGQSYGARQYDLLGTHTQRAIVVLMLTGVPLAFVLAFSGQILVALGQNPEISFQAGLYAQWLIPGLFAYGLLQCLTRFLQTQNIVRILVVSSGLTLLLHIMLCWFLVQSFGLGHKGAALATSISYWFNVALLAIYVKASEAGRRSWHGWSREALKLKDVKLYLWLAIPSTFMTCLEYWAFEMVVLLAGFLPDPKLETSILSISLNTMWMVYTIPSGLSSAISIRVSNELGAGNPQAARLSVYISGIMCLTEGLFVAIITVLVRDIWGYLYSNEEKVVKHVSMMMPILATSDFMDGIQCTLSGAARGCGWQKVCSLINLFAYYVIGLPSAVTFAFVLKIGGKGLWLGIICAMAVQIFALVVMMLRTSWNEEAEKARARVQCSDGSITLD

SEQ ID NO: 82 - DNA, 1761 bp, Paspalum notatum
cagtctcgctcactctaaagtccaaactacgaccagtcaaacaccgacggagagagggcgagaggcacacaccgagagacacgaactagaagagctttgcgtaccatggcgaagggaagcgcggaggaagcgcttctcgccgcggcccaacccgaggaggaccagagcgtgcgggtcgaggtgaagaagcagctatggctggcagggcccatgatctccggcgcgctcctgcagaacgtcatccagatgatctccgtcatgtatgtgggccacctcggcgagctccccctcgccggcgcctccatggccaactccttcgccacggtcacgggcttcagcctgctgcttggaatggccagtgctctggatactctgtgcgggcaagcgttcggagcccggcagtactacctcctgggcatttacaagcaacgtgccatgtttctactcaccctcgtgagcgtccccctcgccgtgatctggttgtacacgggcgagatcctcgccctgctggggcaggaccccgacatcgccgcggaggcaggcaggtacgcacggtggatgatcccagcggtcttcgcctacgggcttctgcagtgccacgtccggttcctgcagacgcagaatgtcgtggtgcccgtgatggcgagcgctggcgcggcggcggggtgccacctggtggtatgctgggcgctcgtgtacgcgctcgggttgggcagcaagggtgcggcgcttagcaacgccatctcgtactgggtcaacgtggccgtgctggccgtgtacgtcagggtgtcgagcacctgcaaggagacgtggactggcttctccacggaggccttccgtgatgctctcggcttcttccggctcgctgtcccatccgctatgatggtctgcttggaaatgtggtcgtttgagctcattgtgcttctatcgggccttctgcctaaccccaaactggagacatccgtgctgtccatcagccttaacactgctgccttcgtgtggatgatcccctttgggcttggctctgccatcagcactcgcgtctccaatgagctcggtgcggggcggccccgagccgctcgccttgcggtgcgcgtagtcctgtttctggccgtctcggaagggctggtgatggggttcatcctcgtctgcgtgcgctacatctggggccacgcgtacagcaacgtcgaggaggtggttacatacgtggccaagatgatgctcgtcattgcggtgtcgaactttttcgatggaatccagtgtgttctttcaggcgtggcaagaggctgtggatggcagaagattggtgcttgcgttaaccttggtgcctactatttggtcggcattccttcagcatacctcatagcttttgttctccgtgttggcgggacgggcctgtggttgggcatcatctgtgggctcttggtgcaagtcctgctgctaatgatcgtcacgctgtgtacgaactgggatagtgaggcaaccaaggcgaaggacagagtttacagttcttctcgtcctgcggattttgaaacatgaccgaatgcagtgtagtgaagaagcgttttaccacacaacctagctggtggaatcccaaagagaagagacacttaacttaattatacttattacagagtttccatcagcattatcaatatgcagttacagattatctaaaataagctttccatgtattctcacaggcaagctgtccagcatacttatgatgtgtactccattttttttgtttgatgccatgca

SEQ ID NO: 83 - protein, 477 aa, Paspalum notatum
MAKGSAEEALLAAAQPEEDQSVRVEVKKQLWLAGPMISGALLQNVIQMISVMYVGHLGELPLAGASMANSFATVTGFSLLLGMASALDTLCGQAFGARQYYLLGIYKQRAMFLLTLVSVPLAVIWLYTGEILALLGQDPDIAAEAGRYARWMIPAVFAYGLLQCHVRFLQTQNVVVPVMASAGAAAGCHLVVCWALVYALGLGSKGAALSNAISYWVNVAVLAVYVRVSSTCKETWTGFSTEAFRDALGFFRLAVPSAMMVCLEMWSFELIVLLSGLLPNPKLETSVLSISLNTAAFVWMIPFGLGSAISTRVSNELGAGRPRAARLAVRVVLFLAVSEGLVMGFILVCVRYIWGHAYSNVEEVVTYVAKMMLVIAVSNFFDGIQCVLSGVARGCGWQKIGACVNLGAYYLVGIPSAYLIAFVLRVGGTGLWLGIICGLLVQVLLLMIVTLCTNWDSEATKAKDRVYSSSRPADFET

SEQ ID NO: 84 - DNA, 1385 bp, Paspalum notatum
aacgtcaccggcttcagccttctctacggcatggcgagcgcgctggacacgctgtgcgggcaggccttcggagccaggcagtacggccgcctgggcatctacaagcagcgagcgatgctggtgctcgcgctcacctgcgttctgatcgccgccgtctgggccaacgccggcaagatcctcgtcctcatcggccaggaccccgacatcgccgccgaggccggcgcctactcacggtggctcataccgtgcctcgtccccttcgtcccgctcgtgtgccacatccgtttcctacaggcccagagcatcgtcgtgccggtaatggtgagctctggtgcgacggcgctgagccacgtcttggtgtgctgggcgctggtgttcaaggccggaatggggagcaaaggcgctgcgctgagcagcgccatttcctacaccatcaacctgaccatgctagctctctacgtcaggctctcaagcgcctgcaggaggacatggactggattctccacggaggctttcagagagcttctccgattcaccgagctcgccatcccgtctgcgatgatggtctgtttggagtggtggtcctttgaactgcttgtgcttctatccggcctactacccaatcctacgcttgaaacttcagtgctgtcaatatgcctcaacaccggtgcactcttgttcatggtaccgtttggtctctgcacagccataagcacacgtgtttcaaatgaacttggtgctggtcagccccaagcagcaaagctagcaactcgtgtggtcatgtttatcgccttatctgcgggcttgctgctcggatctgccatgattttgctacgcaatttctggggttatatgtacagcaatgaacctgaagttgtggcatacattgctagaatgataccggttcttgcaatatcgttcttcacagatgggcttcacagttctctatcaggagtgttgactggatgtggtgagcagaagattggtgcacgtgttaatctcgctgcgttctacttggcgggcattcccatggccgtgttgcttgcatttgtcctccatctaaatggaatgggtctgtggcttggcatcgtttgtggcagcctttccaagcttgtgttgctcatctggatcacaatgcggataaactgggagaaagaggcaatcaaggcaaaagaaacggcttttaattcatctcttccagttacatgacagagttatgagcaaccagacatcaagaccacaacatggcacttgcaaactgagagatattagggttctgtgttatttaaatttaggatatgatgagataagatatgcctctattcatcatagcggtctcatgtttaaacattgtaacttctctattaacttcatcagaatttaac

SEQ ID NO: 85 - protein, 402 aa, Paspalum notatum
NVTGFSLLYGMASALDTLCGQAFGARQYGRLGIYKQRAMLVLALTCVLIAAVWANAGKILVLIGQDPDIAAEAGAYSRWLIPCLVPFVPLVCHIRFLQAQSIVVPVMVSSGATALSHVLVCWALVFKAGMGSKGAALSSAISYTINLTMLALYVRLSSACRRTWTGFSTEAFRELLRFTELAIPSAMMVCLEWWSFELLVLLSGLLPNPTLETSVLSICLNTGALLFMVPFGLCTAISTRVSNELGAGQPQAAKLATRVVMFIALSAGLLLGSAMILLRNFWGYMYSNEPEVVAYIARMIPVLAISFFTDGLHSSLSGVLTGCGEQKIGARVNLAAFYLAGIPMAVLLAFVLHLNGMGLWLGIVCGSLSKLVLLIWITMRINWEKEAIKAKETAFNSSLPVT

SEQ ID NO: 86 - DNA, 2023 bp, Artificial Sequence
  Pn_NODE _22180 N-terminus complete
atggcgaagaagccagtggaggaagcgctcctcgcagcggcagacgagcaggagagcctgagccgccccacggtgggggcggaggcgcggcggcaggtggggctcgcggcgccgctggtggcgtgcagcctgctgcagtacagcttgcaggtggtctccgtcatgttcgccggccacctcggggagctctccctctccggcgcctccgtcgctgcctccttcgccaacgtcaccggcttcagcgtcctgctgggcatggggagcgcattggataccttttgtggacaatcatatggagcaaggcaatatgatctgctcgggacacacacacaaagggctatagttgttcttatgcttacaggtgttcctttggcatttgttttggccttctctggtcaaatcctggttgctcttggtcaaaatcctgaaatatcatttcaagctggactgtacgctcagtggttgatccctggtcttttcgcatacggtttgcttcagtgccttaccagatttctgcagacccaaaatattgtccgaatattggtagtttcctctggacttactttgctacttcacattatgctgtgctggttcctggttcaaagttttggccttggccacaaaggcgcagctctggcgacctcaatatcttactggttcaatgtggcattgctagcaatatatgtgaaagcctctgaagctggcagaagaagctggcatggatggtcaagggaggcactaaagttaaaggatgtaaaactatatctatggttggccattccatctacatttatgacctgcttggagtattgggcatttgagatggtggttctcctagcaggatttcttccagatccaaaactggaaacttcaattttatccatcagcctaaacacaatgtggatggtttatacaattccaagtggcctcagtagtgcaataagtattagagtgtccaatgaactaggtgctgggaacccacaggcagcacgcctgtcagtttatatttcaggaatcatgtgcctaactgaaggcctttttgtagctatcatcacagtattagtgcgagatatctggggttatttgtacagcaatgaagaaaaggtcgtgaagcatgtatcgatgatgatgccaattcttgctacttctgacttcatggatggaatacagtgcacactatcaggcgcggctcgaggatgtggctggcagaaagtatgctcacttatcaacctgtttgcttactatgttattggtctcccttcagctgtcacttttgcatttgttttgaagattggtggtaagggcctttggctgggaattatatgtgctatggcagtgcaaatatttgctttggtagtgatgatgcttcgaaccagctggaatgaagaggctgaaaaggcccgggctagagttcagtgttcagatggcagcattacattggactgaatcttatcagttgtgaattgtgattatagatcccttaaggtgcagtgtataggtttatatgagtcctttgggccaaggcagcagctctggatctggcgggactgagctatcagcctaaaagcaggagtacttggcttgcaattgatggccttggagtacgtttctttctggtttctgcggctcaatgatagcatcgtgaaaggcgaggaagcaaccgaaatttggtagctccggaagtggaggttgtcgtggacgctgagctgtttccaaatcccaaatactccacctgctcaccgtgtgggttaatcgactctcacctgaaagatatttgctattactacaattgatgtttgctctgaaaggaaggaaggagagagggtgtaaacttttcatgctactacgcttcaggagttcaggtgtcagtctgtcagggtgtttccctggaatgaaagcttcagggtgcgctcaggatgccatgtcagctagcggtctgttgcgtgggcgttttatacagagctgatcgatggcatctgcgatcgcgtctgtatagataatgtatggatggctcagaccatcc

SEQ ID NO: 87 - protein, 481 aa, Artificial Sequence
  Pn_NODE21180 N-terminus complete
MAKKPVEEALLAAADEQESLSRPTVGAEARRQVGLAAPLVACSLLQYSLQVVSVMFAGHLGELSLSGASVAASFANVTGFSVLLGMGSALDTFCGQSYGARQYDLLGTHTQRAIVVLMLTGVPLAFVLAFSGQILVALGQNPEISFQAGLYAQWLIPGLFAYGLLQCLTRFLQTQNIVRILVVSSGLTLLLHIMLCWFLVQSFGLGHKGAALATSISYWFNVALLAIYVKASEAGRRSWHGWSREALKLKDVKLYLWLAIPSTFMTCLEYWAFEMVVLLAGFLPDPKLETSILSISLNTMWMVYTIPSGLSSAISIRVSNELGAGNPQAARLSVYISGIMCLTEGLFVAIITVLVRDIWGYLYSNEEKVVKHVSMMMPILATSDFMDGIQCTLSGAARGCGWQKVCSLINLFAYYVIGLPSAVTFAFVLKIGGKGLWLGIICAMAVQIFALVVMMLRTSWNEEAEKARARVQCSDGSITLD

SEQ ID NO: 88 - protein, 488 aa, Panicum virgatum
MGSSPEAPLLPPQRGGEEAGRCRWWRGGASWAAATAEAGRLAALGAPMIAVALLQLMMQLISTVMVGHLGEVPLAGAAIANSLTNVSGFSVLMGLACGLETICGQAYGAEQYHKLALFTYRSIVVLLVASVPIAIIWLFIPDVLPLIGQDAQIASEAGRYALWLIPGLFAYSVAQCLSKFLQSQSLIFPMVLSSLTTLTLFIPLCWFMVYKVGMGNAGAAFAVSICDWVEVTVLGLYIKFSPSCDKTRAAPTWETFRGIGSFMRLAVPSTLMICLEWWSYELLVLVSGNLPNPALETSVLSICISTVVLVYNLPYGIGNAASVRVSNELGAGNPDGARLVVVVSLSIIICTAVLVSVTLLSLRHFIGIAFSNEEEVVNYVTRMVPLLSISVLIDNLQGVLSGISRGCGWQHLGAYVNLGAFYLIGIPVALVLGFAFHLGGAGFWIGMIAGGATQVTLLSVITAMTNWGKMAEKARDRVFEESLPTQAD

SEQ ID NO: 89 - protein, 491 aa, Oryza sativa; identical to SEQ ID NO: 60

SEQ ID NO: 90 - protein, 491 aa, Oryza sativa; identical to SEQ ID NO: 60

SEQ ID NO: 91 - protein, 408 aa, Oryza sativa
MEERIPLLSKRFPADGTAGVGGGREEEGGDRWWSGLAREAGKVGSMALPMAAMSVAQNAVQVASNMMVGHLPGVLPLSASAIATSLASVSGFSLLVGMASGLETLCGQAYGAKQYDKLGVQTYRAIVTLTVVTIPISLLWVFIGKLLTLIGQDPVISHEAGRYIVWLIPGLFAYAVCQPLTKFLQSQSLIFPMLWSSIATLLLHIPLSWLLVFKTSMGFTGAALAISISYWLNTFMLAAYIRFSCSCKVTRSPPTIEAFRGVGLFLRIALPSALMLCFEWWSFEILVLLSGLLPNPELESSVLSICLTTTSLMYTIPYGLGGAASTRVANELGAGNPEGARSAVHLVMSIAGTEAVLVTGMLFAAQRILGYAYSSDEEVVTYFTSMVPFVCISVAADSLQGVLSGYIS

SEQ ID NO: 92 - protein, 408 aa, Oryza sativa; identical to SEQ ID NO: 91

SEQ ID NO: 93 - protein, 408 aa, Panicum virgatum; identical to SEQ ID NO: 91

SEQ ID NO: 94 - protein, 306 aa, Oryza sativa
MEERIPLLSKRFPADGTAGVGGGREEEGGDRWWSGLAREAGKVGSMALPMAAMSVAQNAVQVASNMMVGHLPGVLPLSASAIATSLASVSGFSLLVGMASGLETLCGQAYGAKQYDKLGVQTYRAIVTLTVVTIPISLLWVFIGKLLTLIGQDPVISHEAGRYIVWLIPGLFAYAVCQPLTKFLQSQSLIFPMLWSSIATLLLHIPLSWLLVFKTSMGFTGAALAISISYWLNTFMLAAYIRFSCSCKVTRSPPTIEAFRGVGLFLRIALPSALMLCFEWWSFEILVLLSGLLPNPELESSVLSIW

SEQ ID NO: 95 - protein, 487 aa, Zea mays; identical to SEQ ID NO: 20

SEQ ID NO: 96 - protein, 441 aa, zea mays
MIAVALLQLMMQVISTIMVGHLGEVPLAGAAIAGSLTNVSGFSVLMGLACGLETICGQAFGAEQYHKVALYTYRSIVVLLIASVPMAILWVFLPDVLPLIGQDPQIAIEAGRYALWLIPGLFAFSVAQCLSKFLQSQSLIFPLVLSSLTTLAVFIPLCWFMVYKVGMGNAGAAFAVSICDWVEVTVLGLYIKFSPSCEKTRAPFTWEAFQGIGSFMRLAVPSALMVCLEWWSYELLVLLSGMLPNAALETSVLSICISTVILVYNLPYGIGTAASVRVSNELGAGNPDSARLVVVVALSIIIFTAVLVSVTLLSLRHFIGIAFSNEEEVVNYVTRMVPLLSISVITDNLQGVLSGISRGCGWQHLGAYVNLGAFYLVGIPVALVAGFALHLGGAGFWIGMIAGGATQVTLLTIITAMTNWRKMADKARDRVYEGSLPIQAN

SEQ ID NO: 97 - protein, 483 aa, Oryza sativa
MSGGGGEVEAAAEAAPLLVPHDPQPAVGAEVRRQVGLAAPLVACSLLQYSLQVVSVMFAGHLGELSLSGASVASSFANVTGFSVLLGMGSALDTFCGQSYGAKQYDMLGTHAQRAIFVLMLMGVPLAFVLAFAGQILIALGQNPEISSEAGLYAVWLIPGLFAYGLLQCLTKFLQTQNIVHPLVVCSGATLVIHILLCWVMVHCFDLGNRGAALSISLSYWFNVILLAIYVKVSEVGRRSWPGWSREALKLKDVNMYLRLAIPSTFMTCLEYWAFEMVVLLAGFLPNPKLETSILSISLNTMWMVYTIPSGLSSAISIRVSNELGARNPQAARLSVFVSGIMCLTEGILVAIITVLVRDIWGYLYSNEEEVVKYVAAMMPILALSDFMDGIQCTLSGAARGCGWQKVCSVINLCAYYTIGIPSAVTFAFVLKIDGKGLWLGIICAMTVQILALVVMLLRTSWNEEAEKARARVQGSDGRITLA

SEQ ID NO: 98 - protein, 479 aa, zea mays; identical to SEQ ID NO: 32

SEQ ID NO: 99 - protein, 410 aa, Zea mays
MANSFATVTGLSLLLGMASALDTLCGQAFGARQYYLLGIYKQRAMFLLTLVSVPLSVVWFYTGEILLLFGQDPDIAAEAGTYARWMIPLLFAYGLLQCHVRFLQTQNIVVPVMASAGAAAACHVVVCWALVYALGMGSKGAALSNAISYWVNVAVLSVYVRVSSACKETWTGFSTEAFRDALGFFRLAVPSALMVCLEMWSFELIVLLSGLLPNPKLETSVLSISLNTAAFVWMIPFGLSSAISTRVSNELGAGRPRAARLAVRVVVLLSVAEGLGVGLILVCVRYVWGHAYSNVEEVVTYVANMMLVIAVSNFFDGIQCVLSGVARGCGWQKIGACINLGAYYIVGIPSAYLIAFVLRVGGTGLWLGIICGLIVQLLLLAIITLCTNWDSEATKAKNRVFNSSSPASGT

SEQ ID NO: 100 - protein, 474 aa, Zea mays
MEKKAATTEEPLLAPRSEHTVAAEAKRLLSLAGPLVASCILQNVVQLVSVMFVGHLGELPLAGASLASSLANVTGFSLLVGMASALDTLCGQAFGARQYGLLGLYKQRAMLVLALACVPIAAVWANAGRILILLGQDRDIAAEAGAYSRWLILSLVPYVPLACHVRFLQTQSIVVPVMASSGATALGHVLVCWALVFKAGMGSKGAALSGAISYSVNLAMLALYVRLSSACKRTWTGFSTEAFRDLLRFTELAVPSAMMVCLEWWSFELLVLLSGLLPNPKLETSVLSICLNTGALLFMVPYGLCTAISTRVSNELGAGEPQAARLAARVVMCIALSAGLLLGSTMILLRSFWGYMYSNEPEVVTYIARMMPVLAISFFTDGLHSCLSGVLTGCGRQKIGARVNLGAYYLAGIPMAVLLAFVLHLNGMGLWLGIVCGSLTKLVLLMWITLRINWEKEATNAKETVFSSSLPVAL

SEQ ID NO: 101 - protein, 474 aa, Zea mays; identical to SEQ ID NO: 100

SEQ ID NO: 102 - protein, 380 aa, Zea mays
MIAVALLQLMMQVISTIMVGHLGEVPLAGAAIAGSLTNVSGFSVLMGLACGLETICGQAFGAEQYHKVALYTYRSIVVLLIASVPMAILWVFLPDVLPLIGQDPQIAIEAGRYALWLIPGLFAFSVAQCLSKFLQSQSLIFPLVLSSLTTLAVFIPLCWFMVYKVGMGNAGAAFAVSICDWVEVTVLGLYIKFSPSCEKTRAPFTWEAFQGIGSFMRLAVPSALMVCLEWWSYELLVLLSGMLPNAAQAAHMTTIDDNPQKPKGPHFPGLSKDKISKGLQTLKGKLKPKTEEKISSGNKKTEDETSVSQVDQIKMKYGYANTTNDDSTSVTKMIGNKLQENMKKLEGINLRAADMASGAQSFSTMAKELLRTTKNEKGTS

SEQ ID NO: 103 - DNA, 2038 bp, Arabidopsis thaliana
atggaagatccacttttattgggagatgatcagttaatcactagaaacctcaagtcaacgccgacatggtggatgaattttacggcggagctgaagaacgtcagctctatggcggcgcctatggccaccgtgacagtgtctcaatatctattgcccgtgatctcggtcatggtcgccggccactgcggtgaactccagctgtctggtgtcactcttgccactgctttcgcaaacgtctccggcttcggcatcatggtaattagtttatcctctgtttaaaccacgttcaagatccaagcgaaacttaacaagtcttggaaatttttgcagtatggtttagtgggtgcacttgaaactctatgtggccaagcttatggagcaaaacaatacactaaaatcggaacttacactttctctgcaatagtctcaaacgtacctatagttgttctcatatcgattctctggttttacatggacaaactctttgtttcacttggacaagatcctgacatctccaaggtagctggttcttacgcggtttgtcttataccggcattgttagctcaagcagtgcaacaacctttgactcggtttctccagactcagggtttggttcttcctcttctctactgtgccataaccacccttttattccatataccagtttgtttgattctggtttacgcgtttggtcttggaagcaatggagccgccttggctattggtttgtcttactggtttaatgtcttgattcttgctttatatgtgagattttcaagcgcttgcgagaagactcgcggctttgtatccgatgatttcgtgttgagtgtcaagcagttttttcagtatgggataccatcagcagcaatgacaacgtaaaaacattcatcttcttcatcgttggtgcattcttttctttcttggcttgatttgttttttgtttctatatgcagcatagaatggtcgttgtttgagctccttatcttatcttcaggactcctcccaaacccgaaactcgagacctctgttctttccatttggtaatctatctttctctctggatctttgtctccctttttacatatatgtctagtatatatcaggagggattgtcttaaatatataggtctagacgtctagtgagtaaatattggttaattaaattagatatagtttatcgaattcatatagacagaattgttacgctttactgatgcaatatttttggttgttgttgtttttttaataatagtcttacaacatcatctctccactgtgtcattccaatgggtatcggggctgctggaaggtatgattccgataaaaccgactaaatatttgtttgggaattttcagactagtcacaatgtttactttggcagcacacggatttcaaacgaattgggagcgggaaatccggaggttgctaggctggcagtgtttgccggtattttcctttggttcctagaggctaccatttgtagcacacttctgttcacttgcaaaaatatttttggctacgcgttcagcaatagcaaagaagttgtggactatgtcacggagctatcttcgctgctttgtctttcatttatggtcgatggattttcttcagtgcttgatggtattaagatcaaacccttcaattagtgaatgataaaaaatcctatctcgcgactcaaatatgacttttatgaaaaaggggttgctaggggaagtgggtggcaaaatattggagcttgggcaaatgtggtggcttactatctcctaggagctcctgttggatttttcttaggattttggggtcatatgaacggcaaagggctatggattggtgtgatcgttgggtccactgctcaagggatcatactagctatagtcactgcttgcctgagttgggaggagcaggtcaatagcaatcttaaatatatttttggacatttgatgaatcttttttttaccccatactgaagttgtttacaattggaaattgcaggctgccaaggccagagaaagaatagttggaagaacattggagtga